(19) United States
(12) Patent Application Publication
(10) Pub. No.: US 2007/0015170 A1
(43) Pub. Date: Jan. 18, 2007

(54) Method and kit for detecting a risk of coronary heart disease

(75) Inventors: Jukka T. Salonen, Kuopio (FI); Mia Pirskanen, Kuopio (FI); Pekka Uimari, Kuopio (FI); Ricardo Fuentes, Siilinjarvi (FI); Outi Kontkanen, Kuopio (FI); Juha-Matti Aalto, Siilinjarvi (FI)

Correspondence Address: BIRCH STEWART KOLASCH & BIRCH, PO BOX 747, FALLS CHURCH, VA, 22040-0747, US

(73) Assignee: Oy Jurilab Ltd

(21) Appl. No.: 11/255,042

(22) Filed: Oct. 21, 2005

Related U.S. Application Data
(60) Provisional application No. 60/698,041, filed on Jul. 12, 2005.

(51) Int. Cl. C12Q 1/68; G06F 19/00

(52) U.S. Cl. 435/6; 702/20

(57) ABSTRACT

Genes, SNP markers and haplotypes of susceptibility or predisposition to CHD or CHD death are disclosed. Methods for diagnosis, prediction of clinical course and efficacy of treatments for CHD using polymorphisms in the CHD risk genes are also disclosed. The genes, gene products and agents of the invention are also useful for the prevention and treatment of CHD. Kits are also provided for the diagnosis, selecting treatment and assessing prognosis of CHD.

### BACKGROUND OF THE INVENTION

1. Field of the Invention

The present invention relates generally to the field of diagnosis of coronary heart disease (CHD). More particularly, it provides a method of diagnosing or detecting a predisposition or propensity or susceptibility for CHD death. Specifically, the invention is directed to a method that comprises the steps of providing a biological sample of the subject to be tested and detecting the presence or absence of one or several genomic single nucleotide polymorphism (SNP) markers in the biological sample. Furthermore, the invention utilises both genetic and phenotypic information as well as information obtained by questionnaires to construct a score that provides the probability of developing CHD, particularly CHD death. In addition, the invention provides a kit to perform the method. The kit can be used to set a risk level for CHD or CHD death for targeting of treatment and preventive interventions, such as dietary advice as well as stratification of the subject in clinical trials testing drugs and other interventions. The invention also relates to a method for the treatment of CHD.

2. Description of Related Art

Public Health Significance of CVD and CHD

Cardiovascular Diseases (CVD) (ICD/10 codes 100-199, Q20-Q28) include ischemic (coronary) heart disease (CHD), hypertensive diseases, cerebrovascular disease (stroke) and rheumatic fever/rheumatic heart disease, among others (AHA, 2004). In terms of morbidity, mortality and cost CHD is the most important disease group of CVD. CHD (ICD/10 codes 120-125) includes acute myocardial infarction (AMI), other acute ischemic (coronary) heart disease, angina pectoris; atherosclerotic cardiovascular disease and all other forms of chronic ischemic heart disease (AHA, 2004). AMI and angina pectoris are often caused by coronary atherosclerosis, but not always. Other, often contributory pathophysiologies include coronary thrombosis and contriction or contraction and severe arrhythmias. These may cause a coronary death also without coronary narrowing by atherosclerosis.

In 2001 an estimated 16.6 million—or one-third of total global deaths—resulted from the various forms of CVD (7.2 million due to CHD, 5.5 million to cerebrovascular disease, and an additional 3.9 million to hypertensive and other heart conditions). At least 20 million people survive heart attacks and strokes every year, a significant proportion of them requiring costly clinical care, which puts a huge burden on long-term care resources. It is necessary to recognize that CVD are devastating to men, women and children (ADA, 2004).

Around 80% of all CVD deaths worldwide took place in developing, low and middle-income countries. It is estimated that by 2010, CVD will be the leading cause of death in both developed and developing countries. The rise in CVDs reflects a significant change in dietary habits, physical activity levels, and tobacco consumption worldwide as a result of industrialization, urbanization, economic development and food market globalization (WHO, 2004). This emphasizes the role of relatively modern environmental or behavioral risk factors. However, ethnic differences in the incidence and prevalence of CVD and the enrichment of CVD in families suggest that heritable risk factors play a major role.

In terms of disability measured in disability-adjusted life years (DALYs) CVD caused 9.7% of global DALYs, 20.4% of DALYs in developed countries and 8.3% of DALYs in the developing countries (Murray C J L and Lopez A D, 1997).

On the basis of data from the NHANES III study (1988-1994), it is estimated that in 2001, 64.4 million Americans were affected by some form of CVD, which corresponds to a prevalence of 22.6% (21.5% for males, 22.4% for females). Of these, 13.2 million had CHD (6.4% prevalence).

The cost of CVD in the United States in 2004 is estimated at $368.4 billion ($133.2 billion for CHD, $53.6 billion for stroke, $55.5 billion for hypertensive disease). This figure includes health expenditures (direct costs) and lost productivity resulting from morbidity and mortality (indirect costs) (AHA, 2004).

It is important for the health care system to develop strategies to prevent CHD. Once AMI has manifested clinically, irreversible cell death and tissue damage starts to occur in the myocardial muscle. Unfortunately, the myocardial cells that die cannot be revived or replaced from a stem cell population. Also, a major part of the first clinical manifestations of CHD are sudden deaths. Therefore, it is better to prevent AMI from happening in the first place, i.e. primary prevention. Although we already know of certain clinical risk factors that increase AMI risk, there is an unmet medical need to define the genetic factors involved in AMI to more precisely define disease risk or susceptibility.

CHD: a Polygenic Disease

The etiology and pathophysiology of CVD are complexes, but it is known that major risk factors include unhealthy lifestyles and behaviours and a complex interaction between environmental and genetic factors. The four major CVD risk factors are habitual adverse dietary patterns (primarily high intake of cholesterol and saturated fats), habitual cigarette smoking, dyslipidaemia, indicated by adverse total cholesterol levels, and blood pressure above optimal level (Stamler J et al, 1998). Other well established CVD risk factors are age, male gender, obesity, physical inactivity and diabetes. The role of other emerging risk factors for CVD—thrombogenic factors, homocysteine, markers of inflammation, infection and genetic factors—in risk prediction and management is not established (Wood D, 2001).

A positive family history of premature CHD predicts development of CHD independently of other major CVD risk factors (Sholtz R I et al, 1975; Heller R F and Kelson M C, 1983; Barrett-Connor E and Khaw K, 1984; Colditz G A et al, 1986; Hopkins P N et al, 1988; Myers R H et al, 1990; Colditz G A et al, 1009; Jousilahti P et al, 1996; Boer J M et al, 1999; Li R et al, 2000; Hawe E et al, 2003) and persons with a history of family premature CHD who are otherwise predicted to be at low risk by standard risk factors may have a substantial genetic component for disease development (Heller R F and Kelson M C, 1983; Myers R H et al, 1990). The risk ratio of AMI associated with positive premature CHD of either parent is 1.61 in men and 1.85 in women (Jousilahti P et al, 1996). Further support for the genetic contribution to disease risk comes from twin studies. Marenberg et al, 1994 showed a high concordance for age of onset of CHD. Among men, the relative hazard of death from CHD when one's twin died of CHD before the age of 55 years, as compared with the hazard when one's twin did not die before 55, was 8.1 for monozygotic twins and 3.8 for dizygotic twins. Among women, when one's twin died of CHD before the age of 65 years, the relative hazard was 15.0 for monozygotic twins and 2.6 for dizygotic twins.

At the molecular level, atherosclerosis is a time dependent, multistep process involving the interaction of many different key pathways, including lipoprotein metabolism (Chisolm G M and Steinberg D, 2000), lipoprotein oxidation (Salonen J T et al, 1992), coagulation (Tremoli E et al, 1999) and inflammation (Ross R. 1999). Gene mutations in any of these pathways will only provide a partial contribution to risk. Intermediate phenotypes such as hypertension, diabetes, smoking and obesity interact to modulate risk as will do gene-gene and gene-environment interactions (Stephens J W and Humphries S E, 2003). Not all CHD is polygenic in nature; an exception is familial hypercholesterolaemia (FH) (Civeira F, 2004). The responsible mutations causing FH can now be screened for in high-risk individuals to allow early identification and to target early therapy.

In addition to coronary atherosclerosis, CHD and AMI may be caused by other mechanisms such as thrombosis, vasoconstriction and arrhythmias. Like atherosclerosis, also thrombosis can have many pathways. The role of platelet function, the coagulation and fibrinolytic systems is expected to be larger in coronary thrombosis than atherosclerosis. In addition, etiologies of atherosclerosis and thrombosis interact with each other.

Unlike the rare and severe genetic defects that cause monogenic diseases, the genetic factors that modulate the individual susceptibility to multifactorial diseases such as CVD are common, functionally different, forms of gene polymorphisms, which generally have a modest effect at an individual level but, because of their high carrier frequency in the population, can be associated with a high population attributable risk. Environmental factors can reveal or facilitate the phenotypic expression of such AMI risk genes.

Although more than a hundred putative gene associations to CHD have been reported, only a handful have been widely replicated (Fuentes R M, 2004, unpublished review). The association of APOA4 (Rewers M et al, 1994; Wong W M et al, 2003), APOB (Chiodini B D et al, 2003, meta-analysis), APOE (Eichner J E et al, 1993; Nakai K et al, 1998; Inbal a et al, 1999; Brscic E et al, 2000; Humphries S E et al, 2001; Baroni M G et al, 2003; Kumar P et al, 2003), F2 (Kim R J and Becker R C, 2003; Burzotta F et al, 2004, both meta-analyses), F5 (Kim R J and Becker R C, 2003, meta-analysis), IL6 (Rundek T et al, 2002; Georges J L et al, 2001; Humphries S E et al, 2001), MMP3 (Terashima M et al, 1999; Rundek T et al, 2002; Beyzade S et al, 2003; NOS3 (Shimasaki Y et al, 1998; Hibi K et al, 1998; Hingorani A D et al, 1999; Park J E et al, 2000; Cine N et al, 2002), PON1 (Serrato M and Marian A J, 1995; Sanghera D K et al, 1997; Salonen J T et al, 1999; Senti M et al, 2001), SERPINE1 (Pastinen T et al, 1998; Gardemann A et al, 1999; Ardissino D et al, 1999; Mikkelsson J et al, 2000; Fu L et al, 2001; Zhan M et al, 2003), and THBD (Norlund L et al, 1997; Wu K K et al, 2001; Li Y H et al, 2002; Park H Y et al, 2002; Chao T H et al, 2004) have been reproduced by independent groups. Interactions between IL6 and MMP3 (Rauramaa R et al, 2000) and between smoking and APOE (Humphries S E et al, 2001), F5 (Holm J et al, 1999), IL6 (Humphries S E et al, 2001), MMP3 (Humphries S E et al, 2002) and PON1 (Sen-Banerjee S et al, 2000) as examples of gene-gene and gene-environment interactions affecting the risk of CHD have also been reported.

Pathophysiology of Coronary Atherosclerosis

Progression of human atherosclerotic lesions: Human atherosclerotic lesions from the coronary arteries and the aorta can be obtained for study as specimens during therapeutic interventions or at autopsy of persons who died suddenly of causes other than disease. According to current histological criteria atherosclerosis in humans may be divided into two broad categories of lesions: minimal lesions and advanced lesions (Stary H C et al, 1994; Stary H C et al, 1995; Stary H C, 2000). In each of these broad categories three characteristic types of lesions are distinguished: types I (initial), II (fatty streak) and III (intermediate, preatheroma) in the minimal lesions; and types IV (atheroma), V (fibroatheroma) and VI (complicated) in the advanced lesions (FIG. 1). The contiguous nature of the histological changes and the time of life at which a specific change predominates indicate that each represents a gradation or stage in a temporal sequence.

Each type of minimal lesion is focal and relatively small, and contains abnormal accumulations of lipoproteins and cholesterol esters. Increased numbers of cells, mainly macrophages, and accumulations of lipid droplets, mainly within macrophages, can be demonstrated microscopically. Changes in the composition of the matrix and disruption of the intimal architecture are minimal or absent. The media adjacent to the lesions is not diseased, nor is the adventitia affected (Stary H C et al, 1994). Atherosclerotic lesions are considered advanced when accumulations of lipid, cells, and matrix components, including minerals, are associated with structural disorganization, repair and thickening of the intima, as well as deformity of the arterial wall (Stary H C et al, 1995).

It has been found that the arterial intima is thicker in highly susceptible locations from birth. This process is called adaptive intimal thickening, which develops in response to normal asymmetries in fluid mechanical forces to maintain an optimal flow equally at all points along the course of an artery. The thickenings begin to develop in foetal life and are found in everyone at birth (Stary H C et al, 1992). A thick intima is seen at and near bifurcations of arteries and at the mouths of small branch vessels, where it is focal and eccentric. The initial accumulations of lipid and macrophage foam cell in early life are more prominent in a subset of adaptive intimal thickenings. A distinct fluid mechanical force in such locations is low shear stress (Stary H C et al, 1992). In regions of low shear, circulating plasma particles are in longer contact with the endothelial surface. This enhances the frequency with which particles enter the intima. When plasma is too rich in lipoprotein, it accumulates most in these locations. If atheromas are present in later life, they are found here first.

Type I and II lesions are the only ones that occur in infants and children, although they also occur in adults. Type III lesions may evolve soon after puberty and type IV lesions are frequent from the third decade onward. After the third decade of life, lesions of type V and VI composition begin to appear. In middle-aged and older persons, these often become the predominant lesion types (Stary H C et al, 1995; Stary H C, 2000). Type V and VI lesions develop and progress by mechanisms that are, for the most part, different from and superimposed on the continuing lipid accumulation that produced lesion types I through IV (Stary H C et al, 1994; Stary H C et al, 1995; Steinberg D and Lewis A, 1997; Chisolm G M and Steinberg D, 2000). In type IV lesions disarrangement of intimal structure is caused almost solely by an extensive accumulation of extracellular lipid localized in the deep intima (the lipid core). In type V lesions the intima is thickened by substantial reparative fibrous tissue layers. Surface defects, hematoma and thrombotic deposits (type VI lesions) further damage, deform and thicken the intima and accelerate the conversion from clinically silent to overt disease.

The clinical significance of lesion types I, II, and III lies in their role as the silent precursors of possible future disease and their potential for reversibility. Recognition of the period of life in which type III lesions begin should lead to concentrated preventive measures at, or preferably before, that age (Stary H C et al, 1995; Stary H C, 2000). Morbidity and mortality from atherosclerosis is largely due to type IV and type V lesions in which disruptions of the lesion surface, hematoma or hemorrhage, and thrombotic deposits have developed (type VI lesions), which have their clinical correlate as acute ischemic coronary syndromes (Davies M J, 1990; Libby P, 1995).

Molecular biology of human atherosclerotic lesions: The earliest changes that precede the formation of lesions of atherosclerosis take place in the endothelium. These changes include increased endothelial permeability to lipoproteins and other plasma constituents, which is mediated by nitric oxide, prostacyclin, platelet-derived growth factor, angiotensin II, and endothelin; up-regulation of leukocyte adhesion molecules, including L-selectin, integrins, and platelet-endothelial-cell adhesion molecule 1, and the up-regulation of endothelial adhesion molecules, which include E-selectin, P-selectin, intercellular adhesion molecule 1, and vascular-cell adhesion molecule 1; and migration of leukocytes into the artery wall, which is mediated by oxidized low-density lipoprotein, monocyte chemotactic protein 1, interleukin-8, platelet-derived growth factor, macrophage colony-stimulating factor, and osteopontin (Ross R, 1999).

Fatty streaks initially consist of lipid-laden monocytes and macrophages (foam cells) together with T lymphocytes. Later they are joined by various numbers of smooth-muscle cells. The steps involved in this process include smooth-muscle migration, which is stimulated by platelet-derived growth factor, fibroblast growth factor 2, and transforming growth factor β; T-cell activation, which is mediated by tumor necrosis factor α, interleukin-2, and granulocyte-macrophage colony-stimulating factor; foam-cell formation, which is mediated by oxidized low-density lipoprotein, macrophage colony-stimulating factor, tumor necrosis factor α, and interleukin-1; and platelet adherence and aggregation, which are stimulated by integrins, P-selectin, fibrin, thromboxane A2, tissue factor, and the factors described above responsible for the adherence and migration of leukocytes (Ross R, 1999).

As fatty streaks progress to intermediate and advanced lesions, they tend to form a fibrous cap that walls off the lesion from the lumen. This represents a type of healing or fibrous response to the injury. The fibrous cap covers a mixture of leukocytes, lipid, and debris, which may form a necrotic core. These lesions expand at their shoulders by means of continued leukocyte adhesion and entry caused by the same factors as those participating in endothelial dysfunction and fatty-streak formation. The principal factors associated with macrophage accumulation include macrophage colony-stimulating factor, monocyte chemotactic protein 1, and oxidized low-density lipoprotein. The necrotic core represents the results of apoptosis and necrosis, increased proteolytic activity, and lipid accumulation. The fibrous cap forms as a result of increased activity of platelet-derived growth factor, transforming growth factor βinterleukin-1, tumor necrosis factor α, and osteopontin and of decreased connective-tissue degradation (Ross R, 1999).

Rupture of the fibrous cap or ulceration of the fibrous plaque can rapidly lead to thrombosis and usually occurs at sites of thinning of the fibrous cap that covers the advanced lesion. Thinning of the fibrous cap is apparently due to the continuing influx and activation of macrophages, which release metalloproteinases and other proteolytic enzymes at these sites. These enzymes cause degradation of the matrix, which can lead to hemorrhage from the vasa vasorum or from the lumen of the artery and can result in thrombus formation and occlusion of the artery (Ross R, 1999).

Genome-Wide Scan Studies in CHD

The use of genetic marker maps is based on the concept that a gene marker will segregate from a generation to another with a locus causing CHD/AMI. The sections of chromosome inherited in a family are large, typically cMs. Theoretically, a microsatellite marker map should contain 5,000-10,000 markers to cover the entire genome. In practice, genome-wide scans (GWS) family studies have used typically 400 markers or so, which is insufficient to find the majority of disease genes.

A total of 6 GWS studies in CHD (Pajukanta P et al, 2000; Francke S et al, 2001; Broeckel U et al, 2002; Harrap S B et al, 2002; Wang Q et al, 2004; Fox C S et al, 2004) and one meta-analysis including data from 4 original GWS (Chiodini B D and Lewis C M, 2003) have been

TABLE 1
Genome wide scans carried out previously in coronary heart disease.

| | | | Chiodini BD & | Harrap SB et | | |
|---|---|---|---|---|---|---|
| | Fox CS et al. | Wang Q et al. | Lewis CM | al. | Francke S et al. | Pajukanta P et al. |
| Publication | February, 2004 | February, 2004 | October, 2003 | May, 2002 | November, 2001 | December, 2000 |
| year | | | | | | |
| Population | US | US | DE, FI, AU, MU | AU | MU | FI |
| Individuals | 1225 | 1613 | ND | 213 | 535 | 364 |
| Families | 311 | 428 | 807 | 61 sib pairs | 99 | 156 |
| Phenotype | ICA/CCA IMT | CAD/AMI | CHD | ACS | CHD | CHD |
| Marker density | ˜400 | 408 | 303-400 | 400 | 403 | 303-385 |
| Chromosome | | | | | | |
| 1 | | 1p36, 1p21 | | | | |
| 2 | | 2p11 | 2q34-37 | 2q36-q37 | | 2q22-q23 |
| 3 | | 3p24 | 3q26-q27 | 3q26-q27 | 3q27-q29 | |
| 4 | | 4p16, 4q31, 4q32 | | | | |
| 5 | | 5p15, 5p14, | | | | |
| | | 5p12, | | | | |
| | | 5q14, 5q22 | | | | |
| 7 | | 7q22 | | | | |
| 8 | | | | | 8q23 | |
| 9 | | 9p21 | | | | |
| 10 | | 10p14, 10q21 | | | 10q23 | |
| 11 | | 11q12, 11q13, | | | | |
| | | 11q23 | | | | |
| 12 | 12q24 | 12q24 | | | | |
| 13 | | 13q32 | | | | |
| 14 | | 14q22, 14q24 | | | | |
| 16 | | 16q11 | | | 16p13 | |
| 20 | | 20p12 | | 20q11-q13 | | |
| 21 | | 21q22 | | | | |
| X | | | | | | Xq23-q25 |

reported to date. Most of these studies have considered chronic CHD survivors, thus are prone to survival bias (see table 1).

Pajukanta P et al, 2000, found in a linkage study with just over 300 microsatellite markers in 156 families (364 subjects) two large linkage regions in 2q22-q23 and Xq23-q25. The initial linkage regions were 40 cM and 30 cM, respectively. Some fine mapping was done with a very limited number of markers with average 2.5 cM marker interval. Candidate genes in the strongest linkage regions were speculated.

In a larger linkage study using 408 microsatellite markers in 1613 subjects from 428 American mainly Caucasian extended families, Wang Q et al, 2004, found a novel coronary artery disease (CAD)/AMI susceptibility locus in a very large, 32 cM, region in 1p36.

Other GWS studies have found linkage in 2q36-q37, 3q26-q27 and 20q11-q13 for acute coronary syndrome (ACS)(Harrap S B et al, 2002), 12q24 for carotid intimal medial thickness (Fox C S et al, 2004), 14q32 for AMI (Broeckel U et al, 2002) and 3q27-q29, 8q23, 10q23 and 16p13 for CHD (Francke S et al, 2001). A meta-analysis by Chiodini B D and Lewis C M, 2003, concluded that the genetic regions 2q34-q37 and 3q26-q27 might contain risk genes for CHD.

In summary, previous findings of GWS linkage studies in CHD, AMI or atherosclerosis are inconsistent. Each and every study has detected linkage signals in different chromosomal regions. As the number of gene markers has been small, the regions identified have been large, of many cMs.

Opportunity for Population Genetics

Previous medical research concerning the genetic etiology of CHD and AMI has been based to a large extent on retrospective case-control and family studies in humans and studies in genetically modified animals. As recognized only recently, retrospective case-control studies are prone to survival and selection biases, and they have produced a myriad of biased findings concerning a large number of candidate genes. A commonly used approach is to compare gene expression between affected and unaffected persons. Gene expression studies, which are mostly cross-sectional, cannot however separate cause and consequence. Findings from animal models concerning CHD cannot be generalised to humans, as the pathophysiology in humans is unique. The uselessness of the animal studies is the main reason why genetic epidemiologic studies are the most important means in the clarification of genetic etiologies of human diseases.

Prospective cohort studies in humans overcome these problems. Developments in GWS and sequencing technology and methods of data analysis render now possible the attempt to identify liability genes in complex, multifactorial traits, and to dissect out with new precision the role of genetic predisposition and environment/life style factors in these disorders. Genetic and environmental effects vary over the life span, and only longitudinal studies in genetically informative data sets permit the study of such effects. A major advantage of population genetics approaches in disease gene discovery over other methodologies is that it will yield diagnostic markers which are valid in humans.

Identification of genes causing the major public health problems such as CHD is now enabled by the following recent advances in molecular biology, population genetics and bioinformatics: 1. the availability of new genotyping platforms that will dramatically lower operating cost and increase throughputs; 2. the application of genome scans using dense marker maps (>100.000 markers); 3. data analysis using new powerful statistical methods testing for linkage disequilibrium using haplotype sharing analysis, and 4. the recognition that a smaller number of genetic markers than previously thought is sufficient for genome scans in genetically homogeneous populations.

Traditional GWS using microsatellite markers with linkage analyses have not been successful in finding genes causing common diseases. The failure has in part been due to too small a number of genetic markers used in GWS, and in part due to too heterogeneous study populations. With the advancements of the human genome project and genotyping technology, the first dense marker maps have recently become available for mapping the entire human genome. The microarrays used by Jurilab include probes for over 100,000 single nucleotide polymorphism (SNP) markers. These SNPs form a marker map covering, for the first time, the entire genome tightly enough for the discovery of most disease genes causing AMI.

Genetic Homogeneity of the East Finland Founder Population

Finns descend from two human immigration waves occurring about 4,000 and 2,000 years ago. Both Y-chromosomal haplotypes and mitochondrial sequences show low genetic diversity among Finns compared with other European populations and confirm the long-standing isolation of Finland (Sajantila A et al, 1996). During King Gustavus of Vasa (1523-1560) over 300 years ago, internal migrations created regional subisolates, the late settlements (Peltonen L et al, 1999). The most isolated of these are the East Finns.

The East Finnish population is the most genetically-homogenous population isolate known that is large enough for effective gene discovery program. The reasons for homogeneity are: the young age of the population (fewer generations); the small number of founders; long-term geographical isolation; and population bottlenecks because of wars, famine and fatal disease epidemics.

Owing to the genetic homogeneity of the East Finland population there are fewer mutations and haplotypes in important disease predisposing genes and the affected individuals share similar genetic background. Because of the stronger linkage disequilibrium (LD) fewer SNPs and fewer subjects are needed for GWS studies.

### SUMMARY OF THE INVENTION

The present invention relates to single nucleotide polymorphism (SNP) markers, combinations of such markers and haplotypes associated with risk of CHD or CHD death and genes associated with CHD or CHD death within or close to which the said markers or haplotypes formed by some of these markers are located. The said SNP markers may be associated either with increased CHD or CHD death risk or reduced CHD or CHD death risk i.e. protective of CHD or CHD death. The “prediction” or risk implies here that the risk is either increased or reduced.

Thus the present invention provides individual SNP markers associated with CHD or CHD death and combinations of SNP markers and haplotypes in genetic regions associated with CHD or CHD death, genes previously known in the art, but not known to be associated with CHD or CHD death, methods of estimating susceptibility or predisposition of an individual to CHD or CHD death, as well as methods for prediction of clinical course and efficacy of treatments for AMI using polymorphisms in the CHD risk genes. Accordingly the present invention provides novel methods and compositions based on the disclosed CHD or CHD death associated SNP markers, combinations of SNP markers, haplotypes and genes.

The invention further relates to a method for estimating susceptibility or predisposition of an individual to CHD or CHD death comprising the detection of the presence of SNP markers and haplotypes or an alteration in expression of an CHD risk gene set forth in tables 3 through 8, as well as alterations in the polypeptides encoded by the said CHD risk genes. The alterations may be quantitative, qualitative, or both.

The invention yet further relates to a method for estimating susceptibility or predisposition of an individual to CHD or CHD death. The method for estimating susceptibility or predisposition of an individual to CHD or CHD death is comprised of detecting the presence of at-risk haplotypes in an individual's nucleic acid.

The invention further relates to a kit for estimating susceptibility to CHD or CHD death in an individual comprising wholly or in part: amplification reagents for amplifying nucleic acid fragments containing SNP markers, detection reagents for genotyping SNP markers and interpretation software for data analysis and risk assessment.

In one aspect, the invention relates to methods of diagnosing a predisposition to CHD or CHD death. The methods of diagnosing a predisposition to CHD or CHD death in an individual include detecting the presence of SNP markers predicting CHD or CHD death, as well as detecting alterations in expression of genes which are associated with said markers. The alterations in expression can be quantitative, qualitative, or both.

A further object of the present invention is a method of identifying the risk of CHD or CHD death by detecting SNP markers in a biological sample of the subject. The information obtained from this method can be combined with other information concerning an individual, e.g. results from blood measurements, clinical examination and questionnaires. The blood measurements include but are not restricted to the determination of plasma or serum cholesterol and high-density lipoprotein cholesterol. The information to be collected by questionnaire includes information concerning gender, age, family and medical history such as the family history of CHD and diabetes. Clinical information collected by examination includes e.g. information concerning height, weight, hip and waist circumference, systolic and diastolic blood pressure, and heart rate.

The methods of the invention allow the accurate evaluation of CHD death risk at or before CHD onset, thus reducing or minimizing the debilitating effects of CHD. The method can be applied in persons who are free of clinical symptoms and signs of CHD, in those who already have clinical CHD, in those who have family history of CHD or in those who have elevated level or levels of risk factors of CHD.

The invention further provides a method of diagnosing susceptibility to CHD or CHD death in an individual. This method comprises screening for at-risk haplotypes that predict CHD death that are more frequently present in an individual susceptible to CHD death, compared to the frequency of its presence in the general population, wherein the presence of an at-risk haplotype is indicative of a susceptibility to CHD death. The “at-risk haplotype” may also be associated with a reduced rather than increased risk of CHD or CHD death. An “at-risk haplotype” is intended to embrace one or a combination of haplotypes described herein over the markers that show high correlation to CHD or CHD death. Kits for diagnosing susceptibility to CHD or CHD death in an individual are also disclosed.

Those skilled in the art will readily recognize that the analysis of the nucleotides present in one or several of the SNP markers of this invention in an individual's nucleic acid can be done by any method or technique capable of determining nucleotides present in a polymorphic site. As it is obvious in the art the nucleotides present in SNP markers can be determined from either nucleic acid strand or from both strands.

The major application of the current invention involves prediction of those at higher risk of developing CHD or dying of CHD. Diagnostic tests that define genetic factors contributing to CHD or CHD death might be used together with or independent of the known clinical risk factors to define an individual's risk relative to the general population. Better means for identifying those individuals at risk for CHD or CHD death should lead to better preventive and treatment regimens, including more aggressive management of the current clinical risk factors such as cigarette smoking, hypercholesterolemia, elevated LDL cholesterol, low HDL cholesterol, hypertension and elevated blood pressure, diabetes mellitus, glucose intolerance, insulin resistance and the metabolic syndrome, obesity, lack of physical activity, and inflammatory components as reflected by increased C-reactive protein levels or other inflammatory markers. Information on genetic risk may be used by physicians to help convince particular patients to adjust life style (e.g. to stop smoking, reduce caloric intake, to increase exercise).

A further object of the invention is to provide a method for the selection of human subjects for studies testing anticoronary and antihypertensive effects of drugs.

Another object of the invention is a method for the selection of subjects for clinical trials testing anticoronary and antihypertensive drugs.

Still another object of the invention is to provide a method for prediction of clinical course and efficacy of treatments for CHD using polymorphisms in the CHD risk genes. The genes, gene products and agents of the invention are also useful for treating other related clinical or coronary events such as angina pectoris, and chronic CHD for monitoring the effectiveness of their treatment, and for drug development. Kits are also provided for the diagnosis, treatment and prognosis of CHD.

A further object of the invention is a method for treating CHD in a subject with CHD by influencing the DNA sequence, expression or proteins of any of the genes of the invention in a human or animal subject. A related object of the invention is a method for preventing the onset of CHD in a subject by influencing the DNA sequence, expression or proteins of any of the genes of the invention in a human or animal subject.

### BRIEF DESCRIPTION OF THE DRAWING

FIG. 1, illustrates the pathways in the evolution and progression of human atherosclerotic lesions (from Stary H C et al., 1995, without modification).

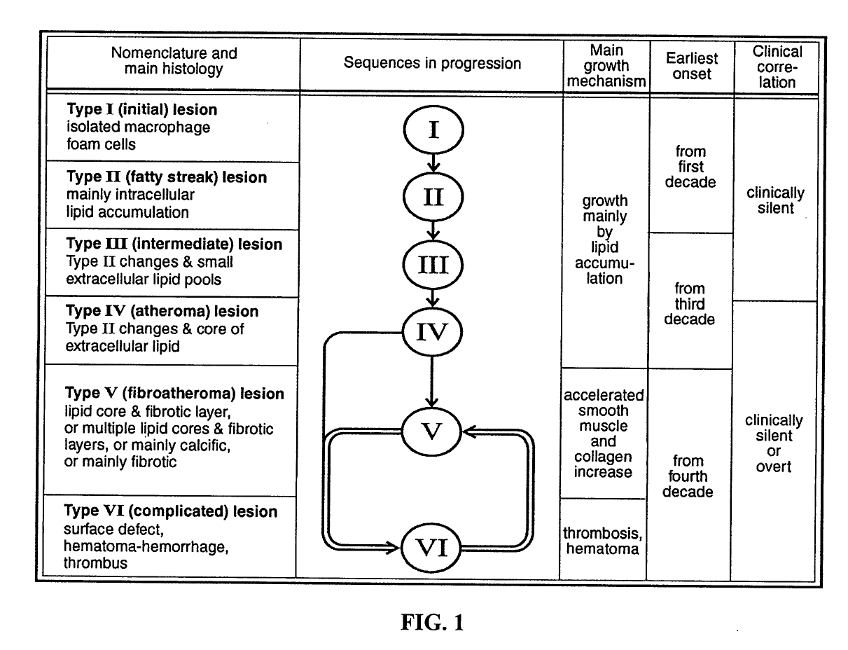

### DETAILED DESCRIPTION OF THE INVENTION

Representative Target Population

An individual at risk of CHD or CHD death is an individual who has at least one risk factor, such as personal history of AMI, family history of AMI, cigarette smoking, hypercholesterolemia, elevated LDL cholesterol, low HDL cholesterol, hypertension and elevated blood pressure, diabetes mellitus, glucose intolerance, insulin resistance and the metabolic syndrome, obesity, lack of physical activity, elevated inflammatory marker, and an at-risk allele or haplotype with one or several CHD risk SNP markers.

In another embodiment of the invention, an individual who is at risk of CHD death is an individual who has a risk-increasing allele in an CHD risk gene, in which the presence of the polymorphism is indicative of a susceptibility to CHD or CHD death. The term “gene,” as used herein, refers to an entirety containing all regulatory elements located both upstream and downstream as well as within of a polypeptide encoding sequence, 5′ and 3′ untranslated regions of mRNA and the entire polypeptide encoding sequence including all exon and intron sequences (also alternatively spliced exons and introns) of a gene.

Assessment for at-Risk Alleles and at-Risk Haplotypes

The genetic markers are particular “alleles” at “polymorphic sites” associated with CHD or CHD death. A nucleotide position at which more than one sequence is possible in a population, is referred to herein as a “polymorphic site”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene. Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences.

Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, aminoacid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a CHD death susceptibility gene. For example, a nucleotide change resulting in a change in polypeptide sequence can alter the physiological properties of a polypeptide dramatically by resulting in altered activity, distribution and stability or otherwise affect on properties of a polypeptide.

Alternatively, nucleotide sequence variants can result in changes affecting transcription of a gene or translation of its mRNA. A polymorphic site located in a regulatory region of a gene may result in altered transcription of a gene e.g. due to altered tissue specifity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of a CHD susceptibility gene.

A “haplotype,” as described herein, refers to any combination of genetic markers (“alleles”), such as those set forth in tables 4, 5, 7 and 8. A haplotype can comprise two or more alleles.

As it is recognized by those skilled in the art the same haplotype can be described differently by determining the haplotype defining alleles from different strands e.g. the haplotype ACGT, defined by SNP markers rs9307776 (A/T) (SEQ ID NO:1427), rs10516735 (C/T) (SEQ ID NO:390), rs2055178 (A/G) (SEQ ID NO:754) and rs1353387(C/T) (SEQ ID NO:500) described in this invention is the same as haplotype TGCA defined by the same SNP markers which have been determined from the other strand rs9307776 (T/A) (SEQ ID NO:1427), rs10516735 (G/A) (SEQ ID NO:390), rs2055178 (T/C) (SEQ ID NO:754) and rs1353387 (G/A) (SEQ ID NO:500) or haplotype TCGT based on rs9307776 (T/A) (SEQ ID NO:1427), rs10516735 (C/T) (SEQ ID NO:390), rs2055178 (A/G) (SEQ ID NO:754) and rs1353387 (C/T) (SEQ ID NO:500), in which the first SNP marker is determined from the other strand.

The haplotypes described herein, e.g., having markers such as those shown in tables 4, 5, 7 and 8 are found more frequently in individuals with high CHD death risk than in individuals with low CHD death risk. Therefore, these haplotypes have predictive value for detecting CHD death risk or a susceptibility to CHD death in an individual. Therefore, detecting haplotypes can be accomplished by methods known in the art for detecting sequences at polymorphic sites. The haplotypes and SNP markers are also associated with the risk of other manifestations of CHD such as chronic CHD and AMI (juha-matti: tähän viittaus USA AMI pat hakemukseen)

It is understood that the CHD death associated at-risk alleles and at-risk haplotypes described in this invention may be associated with other “polymorphic sites” located in CHD death associated genes of this invention. These other CHD death associated polymorphic sites may be either equally useful as genetic markers or even more useful as causative variations explaining the observed association of at-risk alleles and at-risk haplotypes of this invention to CHD or CHD death.

In certain methods described herein, an individual who is at risk for CHD or CHD death is an individual in whom an at-risk allele or an at-risk haplotype is identified. In one embodiment, the at-risk allele or the at-risk haplotype is one that confers a significant risk of CHD death. In one embodiment, significance associated with an allele or a haplotype is measured by an odds ratio. In a further embodiment, the significance is measured by a percentage. In one embodiment, a significant risk is measured as odds ratio of at least about 1.2, including by not limited to: 0.1, 0.2, 0.3, 0.4, 0.5, 0.6, 0.7, 0.8, 0.9, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the allele or the haplotype, and often, environmental factors.

An at-risk haplotype in, or comprising portions of, the CHD risk gene, is one where the haplotype is more frequently present in an individual at risk for CHD death (affected), compared to the frequency of its presence in a healthy individual (control), and wherein the presence of the haplotype is indicative of CHD death risk or susceptibility to CHD death.

In a preferred embodiment, the method comprises assessing in an individual the presence or frequency of SNPs in, comprising portions of, a CHD risk gene, wherein an excess or higher frequency of the SNPs compared to a healthy control individual is indicative that the individual has CHD death risk, or is susceptible to CHD death. See, for example, tables 4, 5, 7 and 8 for SNPs that can form haplotypes that can be used as screening tools. These SNP markers can be identified in at-risk haploptypes. For example, an at-risk haplotype can include microsatellite markers and/or SNPs such as those set forth in tables 4, 5, 7 and 8. The presence of the haplotype is indicative of CHD death risk, or a susceptibility to CHD death, and therefore is indicative of an individual who falls within a target population for the treatment methods described herein.

Consequently, the method of the invention is particularly directed to the detection of one or several of the SNP markers defining the following at-risk haplotypes indicative of CHD death:

1) rs2796249 (C/T) (SEQ ID NO:924), rs6667619 (G/T) (SEQ ID NO:1173), rs1932818 (C/T) (SEQ ID NO:711) and rs6663269 (C/G) (SEQ ID NO:1172) defining the haplotype CTCC;

2) rs6663269 (C/G) (SEQ ID NO:1172), rs1160530 (C/T) (SEQ ID NO:456) and rs631802 (A/G) (SEQ ID NO:1151) defining the haplotype CCG;

3) rs6424260 (A/G) (SEQ ID NO:1152), rs6673130 (A/G) (SEQ ID NO:1174), rs7534667 (C/G) (SEQ ID NO:1292) and rs3766476 (C/T) (SEQ ID NO:1011) defining the haplotype AGGC;

4) rs10489416 (C/T) (SEQ ID NO:78), rs2202094 (G/T) (SEQ ID NO:800), rs218390 (A/T) (SEQ ID NO:790), rs218385 (C/T) (SEQ ID NO:789) and rs218381 (A/G) (SEQ ID NO:788) defining the haplotype TGTTA;

5) rs10520241 (C/T) (SEQ ID NO:420), rs6723256 (C/G) (SEQ ID NO:1179), rs1430635 (A/G) (SEQ ID NO:553) and rs1864549 (G/T) (SEQ ID NO:672) defining the haplotype TCGG;

6) rs1228055 (A/G) (SEQ ID NO:460), rs1228054 (C/T) (SEQ ID NO:459), rs1922035 (C/T) (SEQ ID NO:705) and rs6746500 (C/T) (SEQ ID NO:1182) defining the haplotype ATCT;

7) rs2345512 (C/G) (SEQ ID NO:839), rs7570727 (A/G) (SEQ ID NO:1297), rs2345516 (C/T) (SEQ ID NO:840) and rs2345518 (A/C) (SEQ ID NO:842) defining the haplotype CGCA;

8) rs10495493 (A/G) (SEQ ID NO:153), rs1902021 (A/G) (SEQ ID NO:695), rs722087 (A/G) (SEQ ID NO:1253) and rs962580 (C/G) (SEQ ID NO:1473) defining the haplotype GAAC;

9) rs10511164 (A/G) (SEQ ID NO:328), rs832064 (A/T) (SEQ ID NO:1370), rs937128 (C/T) (SEQ ID NO:1454) and rs1546223 (C/T) (SEQ ID NO:610) defining the haplotype AATT;

10) rs4686145 (A/C) (SEQ ID NO:1083), rs6768216 (C/T) (SEQ ID NO:1186), rs162803 (C/G) (SEQ ID NO:644) and rs10514663 (C/T) (SEQ ID NO:359) defining the haplotype CTGC;

11) rs2201151 (G/T) (SEQ ID NO:799), rs4857302 (A/C) (SEQ ID NO:1100), rs1492054 (C/T) (SEQ ID NO:586) and rs10511164 (A/G) (SEQ ID NO:328) defining the haplotype GCCA;

12) rs223921 (C/G) (SEQ ID NO:813) and rs10489033 (A/G) (SEQ ID NO:73) defining the haplotype CA;

13) rs2703134 (C/G) (SEQ ID NO:911), rs2703133 (C/G) (SEQ ID NO:910), rs2703132 (C/G) (SEQ ID NO:909), rs2703137 (G/T) (SEQ ID NO:912) and rs2645690 (C/G) (SEQ ID NO:907) defining the haplotype GCCTG;

14) rs9307776 (A/T) (SEQ ID NO:1427), rs10516735 (C/T) (SEQ ID NO:390), rs2055178 (A/G) (SEQ ID NO:754) and rs1353387 (C/T) (SEQ ID NO:500) defining the haplotype ACGT;

15) rs10520435 (C/T) (SEQ ID NO:423), rs1379987 (A/G) (SEQ ID NO:519) and rs2100684 (C/T) (SEQ ID NO:766) defining the haplotype CAC;

16) rs409336 (A/C) (SEQ ID NO:1044), rs2472649 (C/T) (SEQ ID NO:887), rs450373 (A/G) (SEQ ID NO:1065) and rs484608 (A/G) (SEQ ID NO:1099) defining the haplotype ACAA;

17) rs10516922 (C/T) (SEQ ID NO:392), rs10516923 (A/G) (SEQ ID NO:393), rs10516924 (A/C) (SEQ ID NO:394), rs10516925 (C/T) (SEQ ID NO:395) and rs10516926 (C/T) (SEQ ID NO:396) defining the haplotype CAATC;

18) rs10515605 (A/G) (SEQ ID NO:370), rs7709159 (C/T) (SEQ ID NO:1317) and rs10515609 (C/T) (SEQ ID NO:371) defining the haplotype ACT;

19) rs2301081 (A/C) (SEQ ID NO:832), rs1966580 (C/T) (SEQ ID NO:718), rs2301086 (C/T) (SEQ ID NO:833), rs1346572 (C/T) (SEQ ID NO:497) and rs10514263 (A/C) (SEQ ID NO:355) defining the haplotype ATCTA;

20) rs10515538 (C/T) (SEQ ID NO:366), rs10515541 (C/T) (SEQ ID NO:367), rs10515542 (C/T) (SEQ ID NO:368) and rs358635 (A/G) (SEQ ID NO:995) defining the haplotype CTCA;

21) rs207098 (A/C) (SEQ ID NO:757), rs207097 (A/G) (SEQ ID NO:756) and rs10484411 (A/G) (SEQ ID NO:46) defining the haplotype CGG;

22) rs7766687 (C/T) (SEQ ID NO:1323), rs6922836 (G/T) (SEQ ID NO:1202), rs10498950 (C/T) (SEQ ID NO:184) and rs6934503 (C/G) (SEQ ID NO:1203) defining the haplotype TTTG;

23) rs9297050 (A/G) (SEQ ID NO:1414), rs2206144 (C/T) (SEQ ID NO:803), rs4716220 (A/G) (SEQ ID NO:1084) and rs214614 (A/G) (SEQ ID NO:775) defining the haplotype GCAA;

24) rs6952184 (C/T) (SEQ ID NO:1205) and rs7807993 (A/C) (SEQ ID NO:1327) defining the haplotype TC;

25) rs10487391 (A/G) (SEQ ID NO:65), rs3757798 (A/G) (SEQ ID NO:1007) and rs3757797 (A/C) (SEQ ID NO:1006) defining the haplotype GAA;

26) rs10499328 (G/T) (SEQ ID NO:187), rs4418248 (C/T) (SEQ ID NO:1062) and rs6952184 (C/T) (SEQ ID NO:1205) defining the haplotype GCT;

27) rs10280843 (A/G) (SEQ ID NO:17), rs10241344 (C/G) (SEQ ID NO:10) and rs13073 (A/G) (SEQ ID NO:469) defining the haplotype GGG;

28) rs1573311 (C/T) (SEQ ID NO:625), rs1037701 (A/C) (SEQ ID NO:25), rs1265145 (C/T) (SEQ ID NO:463), rs1265151 (C/T) (SEQ ID NO:464) and rs10505019 (A/T) (SEQ ID NO:247) defining the haplotype CCCTT;

29) rs4467935 (A/G) (SEQ ID NO:1064), rs10503569 (C/T) (SEQ ID NO:226), rs7819568 (A/G) (SEQ ID NO:1328), rs10503570 (A/G) (SEQ ID NO:227) and rs4240184 (C/T) (SEQ ID NO:1054) defining the haplotype ACAAC;

30) rs10505017 (C/T) (SEQ ID NO:246), rs1111908 (A/C) (SEQ ID NO:452), rs7012174 (C/T) (SEQ ID NO:1216) and rs1573311 (C/T) (SEQ ID NO:625) defining the haplotype TATC;

31) rs4403471 (A/G) (SEQ ID NO:1061), rs4743487 (G/T) (SEQ ID NO:1087), rs10512291 (A/G) (SEQ ID NO:338), rs1337690 (C/G) (SEQ ID NO:489) and rs10512292 (C/T) (SEQ ID NO:339) defining the haplotype AGGGT;

32) rs10491759 (A/T) (SEQ ID NO:114), rs8192981 (C/T) (SEQ ID NO:1364), rs549130 (C/T) (SEQ ID NO:1123), rs1590405 (C/T) (SEQ ID NO:632) and rs489504 (C/T) (SEQ ID NO:1103) defining the haplotype ACCCT;

33) rs1541018 (C/T) (SEQ ID NO:606), rs7897982 (C/T) (SEQ ID NO:1342), rs10508463 (A/G) (SEQ ID NO:278), rs10508464 (A/G) (SEQ ID NO:279) and rs10508465 (C/T) (SEQ ID NO:280) defining the haplotype CCAAC;

34) rs7070112 (A/T) (SEQ ID NO:1225), rs1336507 (G/T) (SEQ ID NO:487), rs1336508 (C/G) (SEQ ID NO:488), rs9325491 (A/G) (SEQ ID NO:1451) and rs877816 (A/G) (SEQ ID NO:1380) defining the haplotype ATCGA;

35) rs10501362 (C/T) (SEQ ID NO:197), rs540505 (A/T) (SEQ ID NO:1120), rs2957177 (C/T) (SEQ ID NO:967) and rs493461 (A/C) (SEQ ID NO:1106) defining the haplotype TATA;

36) rs10501869 (A/G) (SEQ ID NO:199), rs964183 (A/G) (SEQ ID NO:1478), rs10501870 (A/T) (SEQ ID NO:200) and rs964646 (A/G) (SEQ ID NO:1479) defining the haplotype AGTG;

37) rs605954 (A/G) (SEQ ID NO:1144), rs527529 (C/T) (SEQ ID NO:1113), rs590105 (G/T) (SEQ ID NO:1131), rs671544 (A/T) (SEQ ID NO:1178) and rs536412 (C/G) (SEQ ID NO:119) defining the haplotype GTGTC;

38) rs10505953 (G/T) (SEQ ID NO:257), rs976436 (C/T) (SEQ ID NO:1491), rs10505954 (A/G) (SEQ ID NO:258) and rs7296881 (G/T) (SEQ ID NO:1269) defining the haplotype GTGT;

39) rs2961370 (A/G) (SEQ ID NO:968), rs7305762 (C/T) (SEQ ID NO:1272), rs10505838 (G/T) (SEQ ID NO:253) and rs7300261 (A/C) (SEQ ID NO:1271) defining the haplotype ATTA;

40) rs772556 (C/T) (SEQ ID NO:1321), rs699585 (G/T) (SEQ ID NO:1212), rs699586 (C/T) (SEQ ID NO:1213) and rs10506468 (A/G) (SEQ ID NO:263) defining the haplotype TTCG;

41) rs9316159 (C/T) (SEQ ID NO:1441), rs9316160 (A/G) (SEQ ID NO:1442), rs10507537 (A/G) (SEQ ID NO:271) and rs7989399 (C/T) (SEQ ID NO:1349) defining the haplotype TAAT;

42) rs1340313 (C/T) (SEQ ID NO:491), rs1340321 (G/T) (SEQ ID NO:492), rs10507707 (C/T) (SEQ ID NO:272), rs10507708 (A/G) (SEQ ID NO:273) and rs10507710 (A/G) (SEQ ID NO:274) defining the haplotype CGCAG;

43) rs744509 (A/G) (SEQ ID NO:1285), rs744511 (A/G) (SEQ ID NO:1286), rs10483534 (A/G) (SEQ ID NO:30) and rs7148846 (A/C) (SEQ ID NO:1235) defining the haplotype GGAC;

44) rs10483732 (A/G) (SEQ ID NO:39), rs718028 (A/G) (SEQ ID NO:1239) and rs10483734 (C/T) (SEQ ID NO:40) defining the haplotype AGT;

45) rs2181663 (A/G) (SEQ ID NO:787), rs2401841 (C/G) (SEQ ID NO:869), rs10484015 (A/G) (SEQ ID NO:42), rs10484016 (C/T) (SEQ ID NO:43) and rs7350724 (A/G) (SEQ ID NO:1282) defining the haplotype ACGCA;

46) rs2255994 (C/T) (SEQ ID NO:819), rs10506993 (C/G) (SEQ ID NO:264), rs1478199 (C/T) (SEQ ID NO:578) and rs1478200 (A/G) (SEQ ID NO:579) defining the haplotype CCCA;

47) rs10519249 (C/T) (SEQ ID NO:412), rs10519250 (A/G) (SEQ ID NO:413), rs10519251 (A/G) (SEQ ID NO:414), rs2413992 (A/G) (SEQ ID NO:871) and rs2413996 (A/C) (SEQ ID NO:872) defining the haplotype CAGAC;

48) rs10521300 (C/T) (SEQ ID NO:436), rs1112899 (C/T) (SEQ ID NO:454), rs8049155 (C/T) (SEQ ID NO:1356), rs1543921 (A/G) (SEQ ID NO:608) and rs9302658 (C/G) (SEQ ID NO:1420) defining the haplotype TTTGG;

49) rs4783294 (C/T) (SEQ ID NO:1090), rs4783295 (A/G) (SEQ ID NO:1091), rs10492864 (A/T) (SEQ ID NO:124) and rs9319579 (A/C) (SEQ ID NO:1447) defining the haplotype CGTC;

50) rs1486747 (A/G) (SEQ ID NO:583), rs7226036 (A/C) (SEQ ID NO:1254) and rs7212568 (C/T) (SEQ ID NO:1252) defining the haplotype AAT;

51) rs10502297 (A/G) (SEQ ID NO:205) and rs1940693 (C/T) (SEQ ID NO:714) defining the haplotype GC;

52) rs530205 (C/T) (SEQ ID NO:1116), rs646128 (A/C) (SEQ ID NO:1155), rs10502879 (A/G) (SEQ ID NO:216) and rs644731 (C/T) (SEQ ID NO:1154) defining the haplotype TAGC;

53) rs1431844 (C/T) (SEQ ID NO:556), rs10502791 (A/T) (SEQ ID NO:212), rs1431838 (A/G) (SEQ ID NO:554) and rs10502792 (C/G) (SEQ ID NO:213) defining the haplotype CTAC;

54) rs7247641 (C/G) (SEQ ID NO:1261), rs1056176 (G/T) (SEQ ID NO:441), rs2124902 (A/G) (SEQ ID NO:769), rs2262138 (C/T) (SEQ ID NO:822) and rs1004246 (A/G) (SEQ ID NO:2) defining the haplotype GGGCG;

55) rs845607 (C/T) (SEQ ID NO:1376), rs1099620 (C/T) (SEQ ID NO:449) and rs845591 (A/T) (SEQ ID NO:1375) defining the haplotype CTA;

56) rs6088033 (C/T) (SEQ ID NO:1147) and rs1321425 (C/G) (SEQ ID NO:474) defining the haplotype TC;

57) rs6028405 (C/T) (SEQ ID NO:1141), rs2206437 (A/T) (SEQ ID NO:804), rs1569608 (C/T) (SEQ ID NO:622), rs2179443 (C/G) (SEQ ID NO:786) and rs909874 (C/T) (SEQ ID NO:1387) defining the haplotype CACGT;

58) rs2824289 (A/T) (SEQ ID NO:930) and rs208921 (A/G) (SEQ ID NO:761) defining the haplotype AA;

59) rs132183 (C/T) (SEQ ID NO:476), rs720441 (C/G) (SEQ ID NO:1246), rs1983705 (C/T) (SEQ ID NO:722) and rs738743 (G/T) (SEQ ID NO:1283) defining the haplotype CCCT;

60) rs2870458 (G/T) (SEQ ID NO:952) and rs2206024 (C/T) (SEQ ID NO:802) defining the haplotype TT

Monitoring Progress of Treatment

The current invention also pertains to methods of monitoring the effectiveness of a treatment of CHD on the expression (e.g., relative or absolute expression) of one or more CHD risk genes. CHD death susceptibility gene mRNA or polypeptide it is encoding or biological activity of the encoded polypeptide can be measured in a sample of peripheral blood or cells derived therefrom. An assessment of the levels of expression or biological activity of the polypeptide can be made before and during treatment with CHD therapeutic agents.

Alternatively the effectiveness of a treatment of CHD can be followed by monitoring biological networks and/or metabolic pathways related to one or several polypeptides encoded by CHD risk genes listed in table 6. Monitoring of biological networks and/or metabolic pathways can be done e.g. by measuring one or several polypeptides from plasma proteome and/or by measuring one or several metabolites from plasma metabolome before and during treatment. Effectiveness of a treatment is evaluated by comparing observed changes in biological networks and or metabolic pathways following treatment with CHD therapeutic agents to the data available from healthy subjects.

For example, in one embodiment of the invention, an individual who is a member of the target population can be assessed for response to treatment with an CHD inhibitor, by examining CHD risk gene encoding polypeptide biological activity or absolute and/or relative levels of CHD risk gene encoding polypeptide or mRNA in peripheral blood in general or specific cell subfractions or combination of cell subfractions.

In addition, variations such as haplotypes or mutations within or near (within one to hundreds of kb) of the CHD risk gene may be used to identify individuals who are at higher risk for CHD death to increase the power and efficiency of clinical trials for pharmaceutical agents to prevent or treat CHD or their complications. The haplotypes and other variations may be used to exclude or fractionate patients in a clinical trial who are likely to have involvement of another pathway in their CHD in order to enrich patients who have pathways involved that are relevant regarding to the treatment tested and boost the power and sensitivity of the clinical trial. Such variations may be used as a pharmacogenetic test to guide selection of pharmaceutical agents for individuals.

Primers, Probes and Nucleic Acid Molecules

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Such probes and primers include polypeptide nucleic acids (Nielsen P E et al, 1991).

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence.

In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

Antisense nucleic acid molecules of the invention can be designed using the nucleotide sequences of tables 3, 4, 5, 7 and 8, and/or the complement of tables 3, 4, 5, 7 and 8, and/or a portion of tables 3, 4, 5, 7 and 8, and/or the complement of tables 3, 4, 5, 7 and 8, and/or a sequence encoding the amino acid sequences (wherein any one of these may optionally comprise at least one polymorphism as shown in tables 3, 4, 5, 7 and 8) and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

The nucleic acid sequences of the CHD death associated genes described in this invention can also be used to compare with endogenous DNA sequences in patients to identify genetic disorders (e.g., a predisposition for or susceptibility to CHD death), and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Polyclonal and Monoclonal Antibodies

Polyclonal and/or monoclonal antibodies that specifically bind one form of the gene product but not to the other form of the gene product are also provided. Antibodies are also provided that bind a portion of either the variant or the reference gene product that contains the polymorphic site or sites. The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′).sub.2 fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as known by those skilled in the art by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique (Kohler G and Milstein C, 1975), the human B cell hybridoma technique (Kozbor D et al, 1982), the EBV-hybridoma technique (Cole S P et al, 1994), or trioma techniques (Hering S et al, 1988). To produce a hybridoma an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (Bierer B et al, 2002). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide (Hayashi N et al, 1995; Hay B N et al, 1992; Huse W D et al, 1989; Griffiths A D et al, 1993). Kits for generating and screening phage display libraries are commercially available.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue such as blood as part of a test predicting the susceptibility to CHD death or as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include .sup.125I, 131I, 35S or 3H.

Diagnostic Assays

The probes, primers and antibodies described herein can be used in methods of diagnosis of risk of CHD or CHD death or diagnosis of a susceptibility to CHD or CHD death, as well as in kits useful for diagnosis of risk of CHD or CHD death or susceptibility to CHD or CHD death or to a disease or condition associated with CHD.

In one embodiment of the invention, diagnosis of risk or susceptibility to CHD or CHD death (or diagnosis of or susceptibility to a disease or condition associated with CHD death), is made by detecting one or several of at-risk alleles or at-risk haplotypes or a combination of at-risk alleles and at-risk haplotypes described in this invention in the subject's nucleic acid as described herein.

In one embodiment of the invention, diagnosis of risk or susceptibility to CHD or CHD death (or diagnosis of or susceptibility to a disease or condition associated with CHD), is made by detecting one or several of polymorphic sites which are associated with at-risk alleles or/and at-risk haplotypes described in this invention in the subject's nucleic acid. Diagnostically the most useful polymorphic sites are those altering the polypeptide structure of a CHD or CHD death associated gene due to a frame shift; due to a premature stop codon, due to an aminoacid change or due to abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other diagnostically useful polymorphic sites are those affecting transcription of a CHD associated gene or translation of it's mRNA due to altered tissue specifity, due to altered transcription rate, due to altered response to physiological status, due to altered translation efficiency of the mRNA and due to altered stability of the mRNA. The presence of nucleotide sequence variants altering the polypeptide structure of CHD associated genes or altering the expression of CHD associated genes is diagnostic for susceptibility to CHD or CHD death.

For diagnostic applications, there may be polymorphisms informative for prediction of disease risk that are in linkage disequilibrium with the functional polymorphism. Such a functional polymorphism may alter splicing sites, affect the stability or transport of mRNA, or otherwise affect the transcription or translation of the nucleic acid. The presence of nucleotide sequence variants associated with functional polymorphism is diagnostic for susceptibility to CHD death.

While we have genotyped and included a limited number of example SNP markers in the experimental section, any functional, regulatory or other mutation or alteration described above in any of the CHD risk genes identified herein is expected to predict the risk of CHD death.

In diagnostic assays determination of the nucleotides present in one or several of the CHD death associated SNP markers of this invention, as well as polymorphic sites associated with CHD death associated SNP markers of this invention, in an individual's nucleic acid can be done by any method or technique which can accurately determine nucleotides present in a polymorphic site. Numerous suitable methods have been described in the art (Kwok P-Y, 2001; Syvanen A-C, 2001), these methods include, but are not limited to, hybridization assays, ligation assays, primer extension assays, enzymatic cleavage assays, chemical cleavage assays and any combinations of these assays. The assays may or may not include PCR, solid phase step, modified oligonucleotides, labeled probes or labeled nucleotides and the assay may be multiplex or singleplex. As it is obvious in the art the nucleotides present in polymorphic site can be determined from one nucleic acid strand or from both strands.

In another embodiment of the invention, diagnosis of a susceptibility to CHD death can also be made by examining transcription of one or several CHD death associated genes. Alterations in transcription can be analysed by a variety of methods as described in the art, including e.g. hybridization methods, enzymatic cleavage assays, RT-PCR assays and microarrays. A test sample from an individual is collected and the alterations in the transcription of CHD death associated genes are assessed from the RNA present in the sample. Altered transcription is diagnostic for a susceptibility to CHD death.

In another embodiment of the invention, diagnosis of a susceptibility to CHD death can also be made by examining expression and/or structure and/or function of a CHD death susceptibility polypeptide. A test sample from an individual is assessed for the presence of an alteration in the expression and/or an alteration in structure and/or function of the polypeptide encoded by a CHD risk gene, or for the presence of a particular polypeptide variant (e.g., an isoform) encoded by a CHD risk gene. An alteration in expression of a polypeptide encoded by a CHD risk gene can be, for example, an alteration in the quantitative polypeptide expression (i.e., the amount of polypeptide produced); an alteration in the structure and/or function of a polypeptide encoded by a CHD risk gene is an alteration in the qualitative polypeptide expression (e.g., expression of a mutant CHD death susceptibility polypeptide or of a different splicing variant or isoform). In a preferred embodiment, detecting a particular splicing variant encoded by a CHD risk gene, or a particular pattern of splicing variants makes diagnosis of the disease or condition associated with CHD death or a susceptibility to a disease or condition associated with CHD death.

Alterations in expression and/or structure and/or function of a CHD death susceptibility polypeptide can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition of polypeptide by a CHD risk gene in a control sample. A control sample is a sample that corresponds to the test sample (e.g., is from the same type of cells), and is from an individual who is not affected by CHD death. An alteration in the expression or composition of the polypeptide in the test sample, as compared with the control sample, is indicative of a susceptibility to CHD death.

Western blotting analysis, using an antibody as described above that specifically binds to a polypeptide encoded by a mutant CHD risk gene, or an antibody that specifically binds to a polypeptide encoded by a non-mutant gene, or an antibody that specifically binds to a particular splicing variant encoded by a CHD risk gene, can be used to identify the presence in a test sample of a particular splicing variant or isoform, or of a polypeptide encoded by a polymorphic or mutant CHD risk gene, or the absence in a test sample of a particular splicing variant or isoform, or of a polypeptide encoded by a non-polymorphic or non-mutant gene. The presence of a polypeptide encoded by a polymorphic or mutant gene, or the absence of a polypeptide encoded by a non-polymorphic or non-mutant gene, is diagnostic for a susceptibility to CHD death, as is the presence (or absence) of particular splicing variants encoded by a CHD risk gene.

In one embodiment of this method, the level or amount of polypeptide encoded by a CHD risk gene in a test sample is compared with the level or amount of the polypeptide encoded by a CHD risk gene in a control sample. A level or amount of the polypeptide in the test sample that is higher or lower than the level or amount of the polypeptide in the control sample, such that the difference is statistically significant, is indicative of an alteration in the expression of the polypeptide encoded by a CHD risk gene, and is diagnostic for a susceptibility to CHD death. Alternatively, the composition of the polypeptide encoded by a CHD risk gene in a test sample is compared with the composition of the polypeptide encoded by a CHD risk gene in a control sample (e.g., the presence of different splicing variants). A difference in the composition of the polypeptide in the test sample, as compared with the composition of the polypeptide in the control sample, is diagnostic for a susceptibility to CHD death. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to CHD death.

In another embodiment, assessment of the splicing variant or isoform(s) of a polypeptide encoded by a polymorphic or mutant CHD risk gene can be performed. The assessment can be performed directly (e.g., by examining the polypeptide itself), or indirectly (e.g., by examining the mRNA encoding the polypeptide, such as through mRNA profiling). For example, probes or primers as described herein can be used to determine which splicing variants or isoforms are encoded by an CHD risk gene mRNA, using standard methods.

The presence in a test sample of a particular splicing variant(s) or isoform(s) associated with CHD death or risk of CHD death, or the absence in a test sample of a particular splicing variant(s) or isoform(s) not associated with CHD death or risk of CHD death, is diagnostic for a disease or condition associated with a CHD risk gene or a susceptibility to a disease or condition associated with a CHD risk gene. Similarly, the absence in a test sample of a particular splicing variant(s) or isoform(s) associated with CHD death or risk of CHD death, or the presence in a test sample of a particular splicing variant(s) or isoform(s) not associated with CHD death or risk of CHD death, is diagnostic for the absence of disease or condition associated with a CHD risk gene or a susceptibility to a disease or condition associated with a CHD risk gene.

The invention further pertains to a method for the diagnosis and identification of susceptibility to CHD death in an individual, by identifying an at-risk allele or an at-risk haplotype in an CHD risk gene. In one embodiment, the at-risk allele or the at-risk haplotype is an allele or a haplotype for which the presence of the haplotype increases the risk of CHD death significantly. Although it is to be understood that identifying whether a risk is significant may depend on a variety of factors, including the specific disease, the haplotype, and often, environmental factors, the significance may be measured by an odds ratio or a percentage. In a further embodiment, the significance is measured by a percentage. In one embodiment, a significant risk is measured as an odds ratio of 0.8 or less or at least about 1.2, including by not limited to: 0.1, 0.2, 0.3, 0.4, 0.5, 0.6, 0.7, 0.8, 0.9, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, an odds ratio of at least 1.2 is significant. In a further embodiment, an odds ratio of at least about 1.5 is significant. In a further embodiment, a significant increase or decrease in risk is at least about 1.7. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase or reduction in risk is at least about 50%. It is understood however, that identifying whether a risk is medically significant may also depend on a variety of factors, including the specific disease, the allele or the haplotype, and often, environmental factors.

The invention also pertains to methods of diagnosing risk or a susceptibility to CHD death in an individual, comprising screening for an at-risk haplotype in the CHD risk gene that is more frequently present in an individual susceptible to CHD death (affected), compared to the frequency of its presence in a healthy individual (control), wherein the presence of the haplotype is indicative of risk or susceptibility to CHD death. See tables 4, 5, 7 and 8 for SNP markers that comprise haplotypes that can be used as screening tools. SNP markers from these lists represent at-risk haplotypes and can be used to design diagnostic tests for determining a susceptibility to CHD death.

Kits (e.g., reagent kits) useful in the methods of diagnosis comprise components useful in any of the methods described herein, including for example, PCR primers, hybridization probes or primers as described herein (e.g., labeled probes or primers), reagents for genotyping SNP markers, reagents for detection of labeled molecules, restriction enzymes (e.g., for RFLP analysis), allele-specific oligonucleotides, DNA polymerases, RNA polymerases, marker enzymes, antibodies which bind to altered or to non-altered (native) CHD death susceptibility polypeptide, means for amplification of nucleic acids comprising one or several AMI risk genes, or means for analyzing the nucleic acid sequence of one or several CHD risk genes or for analyzing the amino acid sequence of one or several CHD death susceptibility polypeptides, etc. In one embodiment, a kit for diagnosing susceptibility to CHD death can comprise primers for nucleic acid amplification of a region in a CHD risk gene comprising an at-risk haplotype that is more frequently present in an individual susceptible to CHD death. The primers can be designed using portions of the nucleic acids flanking SNPs that are indicative of CHD death.

This invention is based on the principle that one or a small number of genotypings are performed, and the mutations to be typed are selected on the basis of their ability to predict CHD death. For this reason any method to genotype mutations in a genomic DNA sample can be used. If non-parallel methods such as real-time PCR are used, the typings are done in a row. The PCR reactions may be multiplexed or carried out separately in a row or in parallel aliquots.

Thus, the detection method of the invention may further comprise a step of combining information concerning age, gender, the family history of hypertension, diabetes and hypercholesterolemia, and the medical history concerning CVD or diabetes of the subject with the results obtained from step b) of the method (see claim 1) for confirming the indication obtained from the detection step. Said information may also concern hypercholesterolemia in the family, smoking status, CHD in the family, history of CVD, obesity in the family, and waist-to-hip circumference ratio (cm/cm) The detection method of the invention may also further comprise a step determining blood, serum or plasma cholesterol, HDL cholesterol, LDL cholesterol, triglyceride, apolipoprotein B and AI, fibrinogen, ferritin, transferrin receptor, C-reactive protein, serum or plasma insulin concentration.

The score that predicts the probability of CHD death may be calculated using a multivariate failure time model or a logistic regression equation. The results from the further steps of the method as described above render possible a step of calculating the probability of CHD death using a logistic regression equation as follows.

Probability of CHD death=1/[1+e(−(−a+Σ(bi*Xi))], where e is Napier's constant, Xi are variables related to the CHD death, bi are coefficients of these variables in the logistic function, and a is the constant term in the logistic function, and wherein a and bi are preferably determined in the population in which the method is to be used, and Xi are preferably selected among the variables that have been measured in the population in which the method is to be used. Preferable values for bi are between −20 and 20; and for i between 0 (none) and 100,000. A negative coefficient bi implies that the marker is risk-reducing and a positive that the marker is risk-increasing.

Xi are binary variables that can have values or are coded as 0 (zero) or 1 (one) such as SNP markers. The model may additionally include any interaction (product) or terms of any variables Xi, e.g. biXi. An algorithm is developed for combining the information to yield a simple prediction of CHD death as percentage of risk in one year, two years, five years, 10 years or 20 years.

Alternative statistical models are failure-time models such as the Cox's proportional hazards' model, other iterative models and neural networking models.

The test can be applied to test the risk of developing a CHD death in both healthy persons, as a screening or predisposition test and high-risk persons (who have e.g. family history of CHD or elevated serum cholesterol or hypertension or diabetes or any combination of these or elevated level of any other coronary risk factor).

The method can be used in the prediction and early diagnosis of CHD in adult persons, stratification and selection of subjects in clinical trials, stratification and selection of persons for intensified preventive and curative interventions. The aim is to reduce the cost of clinical drug trials and health care.

Pharmaceutical Compositions

The present invention also pertains to pharmaceutical compositions comprising agents described herein, particularly nucleotides in CHD risk genes, and/or comprising other splicing variants encoded by CHD risk genes; and/or an agent that alters (e.g., enhances or inhibits) CHD risk gene expression or CHD susceptibility gene polypeptide activity as described herein. For instance, a polypeptide, protein (e.g., a receptor), an agent that alters a CHD risk gene expression, or a CHD susceptibility polypetide binding agent or binding partner, fragment, fusion protein or prodrug thereof, or a nucleotide or nucleic acid construct (vector) comprising a nucleotide of the present invention, or an agent that alters CHD susceptibility gene polypeptide activity, can be formulated with a physiologically acceptable carrier or excipient to prepare a pharmaceutical composition. The carrier and composition can be sterile. The formulation should suit the mode of administration.

In a preferred embodiment pharmaceutical compositions comprise agent or agents reversing, at least partially, CHD death associated changes in biological networks and/or metabolic pathways related to the CHD death associated genes of this invention (Table 6).

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of CHD, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Methods of Therapy

The present invention encompasses methods of treatment (prophylactic and/or therapeutic) for CHD, such as individuals in the target populations described herein, using a CHD therapeutic agent. A “CHD therapeutic agent” is an agent that alters (e.g., enhances or inhibits) CHD risk affecting polypeptide (enzymatic activity or quantity) and/or an CHD risk gene expression, as described herein (e.g., an agonist or antagonist). CHD therapeutic agents can alter a CHD susceptibility polypeptide activity or nucleic acid expression by a variety of means, such as, for example, by providing additional CHD susceptibility polypeptide or by upregulating the transcription or translation of the CHD risk gene; by altering posttranslational processing of the CHD susceptibility polypeptide; by altering transcription of a CHD risk gene splicing variants; or by interfering with a CHD susceptibility polypeptide activity (e.g., by binding to a CHD susceptibility polypeptide); or by downregulating the transcription or translation of the CHD risk gene, or by inhibiting or enhancing the elimination of a CHD susceptibility polypeptide.

In particular, the invention relates to methods of treatment for CHD or susceptibility to CHD (for example, for individuals in an at-risk population such as those described herein); as well as to methods of treatment for manifestations and subtypes of CHD including but not limited to myocardial infarction, angina pectoris, other chronic CHD, atherosclerosis, acute coronary syndrome (e.g., unstable angina, non-ST-elevation myocardial infarction (NSTEMI) or ST-elevation myocardial infarction (STEMI)), peripheral arterial occlusive disease, cerebrovascular stroke, and complications or sequalae of CHD such as congestive heart failure and cardiac hypertrophy and arrythmias.

Representative CHD Therapeutic Agents Include the Following:

nucleic acids or fragments or derivatives thereof described herein, particularly nucleotides encoding the polypeptides described herein and vectors comprising such nucleic acids (e.g., a gene, cDNA, and/or mRNA, double-stranded interfering RNA, a nucleic acid encoding an CHD susceptibility polypeptide or active fragment or derivative thereof, or an oligonucleotide; for example, tables 3 through 8; other polypeptides (e.g., CHD susceptibility receptors); CHD susceptibility polypeptide binding agents; peptidomimetics; fusion proteins or prodrugs thereof, antibodies (e.g., an antibody to a mutant CHD susceptibility polypeptide, or an antibody to a non-mutant CHD susceptibility polypeptide, or an antibody to a particular splicing variant encoded by a CHD risk gene, as described above); ribozymes; other small molecules; and other agents that alter (e.g., inhibit or antagonize) a CHD risk gene expression or polypeptide activity, or that regulate transcription of a CHD risk gene splicing variants (e.g., agents that affect which splicing variants are expressed, or that affect the amount of each splicing variant that is expressed); and other reagents that alter (e.g. induce or agonize) a CHD risk gene expression or polypeptide activity, or that regulate transcription of a CHD risk gene splicing variants (e.g., agents that affect which splicing variants are expressed, or that affect the amount of each splicing variant that is expressed).

More than one CHD therapeutic agent can be used concurrently, if desired.

The CHD therapeutic agent that is a nucleic acid is used in the treatment of CHD. The term, “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition. In the case of atherosclerosis, “treatment” also refers to a minimization or reversal of the development of plaques. The therapy is designed to alter (e.g., inhibit or enhance), replace or supplement activity of a CHD polypeptide in an individual. For example, a CHD therapeutic agent can be administered in order to upregulate or increase the expression or availability of a CHD risk gene or of specific splicing variants of a CHD susceptibility, gene or, conversely, to downregulate or decrease the expression or availability of a CHD risk gene or specific splicing variants of a CHD risk gene. Upregulation or increasing expression or availability of a native CHD risk gene or of a particular splicing variant could interfere with or compensate for the expression or activity of a defective gene or another splicing variant; downregulation or decreasing expression or availability of a native CHD risk gene or of a particular splicing variant could minimize the expression or activity of a defective gene or the particular splicing variant and thereby minimize the impact of the defective gene or the particular splicing variant.

The CHD therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a CHD susceptibility polypeptide, such as tables 3 through 8 which may optionally comprise at least one polymorphism shown in tables 3 through 8; or another nucleic acid that encodes a CHD susceptibility polypeptide or a splicing variant, derivative or fragment thereof, can be used, either alone or in a pharmaceutical composition as described above. For example, a CHD risk gene or a cDNA encoding a CHD susceptibility polypeptide, either by itself or included within a vector, can be introduced into cells (either in vitro or in vivo) such that the cells produce native CHD susceptibility polypeptide. If necessary, cells that have been transformed with the gene or cDNA or a vector comprising the gene or cDNA can be introduced (or re-introduced) into an individual affected with the disease. Thus, cells which, in nature, lack of a native CHD risk gene expression and activity, or have mutant CHD risk gene expression and activity, or have expression of a disease-associated CHD risk gene splicing variant, can be engineered to express a CHD susceptibility polypeptide or an active fragment of a CHD susceptibility polypeptide (or a different variant of a CHD susceptibility polypeptide). In a preferred embodiment, nucleic acid encoding a CHD susceptibility polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g., an oligonucleotide as described below), can be used in “antisense” therapy, in which a nucleic acid (e.g., an oligonucleotide) which specifically hybridizes to the mRNA and/or genomic DNA of a CHD risk gene is administered or generated in situ. The antisense nucleic acid that specifically hybridizes to the mRNA and/or DNA inhibits expression of the CHD susceptibility polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA which is complementary to a portion of the mRNA and/or DNA which encodes a CHD susceptibility polypeptide. Alternatively, the antisense construct can be an oligonucleotide probe which is generated ex vivo and introduced into cells; it then inhibits expression by hybridizing with the mRNA and/or genomic DNA of a CHD risk gene. In one embodiment, the oligonucleotide probes are modified oligonucleotides which are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA. Additionally, general approaches to constructing oligomers useful in antisense therapy are also described, for example, by van der Krol A R et al, 1988 and Stein C A and Cohen J S, 1988. With respect to antisense DNA, oligodeoxyribonucleotides derived from the translation initiation site, e.g., between the −10 and +10 regions of a CHD risk gene sequence, are preferred.

To perform antisense therapy, oligonucleotides (mRNA, cDNA or DNA) are designed that are complementary to mRNA encoding a CHD susceptibility polypeptide. The antisense oligonucleotides bind to CHD susceptibility mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g., for targeting host cell receptors in vivo), or agents facilitating transport across the cell membrane (Letsinger R L et al, 1989; Lemaitre M et al, 1987) or the blood-brain barrier (Jaeger L B and Banks W A, 2004), or hybridization-triggered cleavage agents (van der Krol A R et al, 1988) or intercalating agents, (Zon G, 1988). To this end, the oligonucleotide may be conjugated to another molecule (e.g., a peptide, hybridization triggered cross-linking agent, transport agent, hybridization-triggered cleavage agent).

The antisense molecules are delivered to cells that express a CHD risk gene in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous CHD risk gene transcripts and thereby prevent translation of the CHD susceptibility mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

An endogenous CHD risk gene expression can be also reduced by inactivating or “knocking out” a CHD risk gene or its promoter using targeted homologous recombination (Smithies O et al, 1985; Thomas K R and Capecchi M R, 1987; Thompson S et al, 1989). For example, a mutant, non-functional CHD risk gene (or a completely unrelated DNA sequence) flanked by DNA homologous to the endogenous CHD risk gene (either the coding regions or regulatory regions of a CHD risk gene) can be used, with or without a selectable marker and/or a negative selectable marker, to transfect cells that express a CHD risk gene in vivo. Insertion of the DNA construct, via targeted homologous recombination, results in inactivation of the CHD risk gene. The recombinant DNA constructs can be directly administered or targeted to the required site in vivo using appropriate vectors, as described above. Alternatively, expression of non-mutant CHD risk gene can be increased using a similar method: targeted homologous recombination can be used to insert a DNA construct comprising a non-mutant, functional CHD risk gene (e.g., any gene shown in tables 3 through 8 which may optionally comprise at least one polymorphism shown in tables 3 through 8), or a portion thereof, in place of a mutant CHD risk gene in the cell, as described above. In another embodiment, targeted homologous recombination can be used to insert a DNA construct comprising a nucleic acid that encodes a CHD susceptibility polypeptide variant that differs from that present in the cell.

Alternatively, an endogenous CHD risk gene expression can be reduced by targeting deoxyribonucleotide sequences complementary to the regulatory region of a CHD risk gene (i.e., the CHD risk gene promoter and/or enhancers) to form triple helical structures that prevent transcription of a CHD risk gene in target cells in the body (Helene C, 1991; Helene C et al, 1992; Maher L J, 1992). Likewise, the antisense constructs described herein, by antagonizing the normal biological activity of one of the CHD proteins, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a CHD mRNA or gene sequence) can be used to investigate role of a CHD risk gene in developmental events, as well as the normal cellular function of a CHD risk gene in adult tissue. Such techniques can be utilized in cell culture, but can also be used in the creation of transgenic animals.

In yet another embodiment of the invention, other CHD therapeutic agents as described herein can also be used in the treatment or prevention of CHD. The therapeutic agents can be delivered in a composition, as described above, or by themshelves. They can be administered systemically, or can be targeted to a particular tissue. The therapeutic agents can be produced by a variety of means, including chemical synthesis; recombinant production; in vivo production, e.g. a transgenic animal (Meade H et al, 1990) and can be isolated using standard means such as those described herein.

A combination of any of the above methods of treatment (e.g., administration of non-mutant CHD susceptibility polypeptide in conjunction with antisense therapy targeting mutant CHD susceptibility mRNA; administration of a first splicing variant encoded by a CHD risk gene in conjunction with antisense therapy targeting a second splicing encoded by a CHD risk gene), can also be used.

This application includes sequence listing and tables that are submitted in electronic form. The sequence listing and tables are submitted herewith on one original and one duplicate compact disc (in compliance with 37 C.F.R. § 1.52(e)) designated respectively as Copy 1 and Copy 2, and labeled in compliance with 37 C.F.R. § 1.52(e)(6). All the material in the sequence listing and tables on compact disc is hereby incorporated in their entirety herein by reference, and identified by the following data of file names, creation date and size in bytes:

| FILE NAME | CREATED | SIZE IN BYTES |
|---|---|---|
| Sequence listing.txt | 08-Aug-05 | 219 000 |
| Table3_CHD.txt | 09-May-05 | 51 000 |
| Table4_CHD.txt | 11-Aug-05 | 103 000 |
| Table5_CHD.txt | 10-May-05 | 28 000 |
| Table6_CHD.txt | 11-Aug-05 | 43 000 |
| Table7_CHD.txt | 25-May-05 | 5 000 |
| Table8_CHD.txt | 02-Jun-05 | 6 000 |
| Table9_CHD.txt | 02-Jun-05 | 5 000 |

The invention will be further described by the following non-limiting examples. The teachings of all publications cited herein are incorporated herein by reference in their entirety.

### EXPERIMENTAL SECTION

East Finnish AMI Patients and Phenotype Characterization

The subjects were participants of the Kuopio Ischaemic Heart Disease Risk Factor Study (KIHD), which is an ongoing prospective population-based study designed to investigate risk factors for chronic diseases, including AMI and CVD, among middle-aged men (Salonen J T 1988, Salonen J T et al 1999, Tuomainen T-P et al 1999). The study population was a random age-stratified sample of men living in Eastern Finland who were 42, 48, 54 or 60 years old at baseline examinations in 1984-1989. A total of 2682 men were examined during 1984-89. The male cohort was complemented by a random population sample of 920 women, first examined during 1998-2001, at the time of the 1-year follow up of the male cohort. The follow-up of coronary events was to the end of 2002, providing a follow-up time ranging from 13 years to 18 years. The recruitment and examination of the subjects has been described previously in detail (Salonen J T 1988). The University of Kuopio Research Ethics Committee approved the study. All participants gave their written informed consent.

Data on CHD and AMI during the follow-up were obtained by computer record linkage to the national computerized hospital discharge registry. Diagnostic information was collected from the hospitals and all heart attacks events were classified according to rigid predefined criteria. The diagnostic classification of acute coronary events was based on symptoms, electrocardiographic findings, cardiac enzyme elevations, autopsy findings and the history of CHD. Each suspected coronary event (ICD-9 codes 410-414 and ICD-10 codes 120-125) was classified into 1) a definite AMI, 2) a probable AMI, 3) a typical acute chest pain episode of more than 20 minutes indicating CHD, 4) an ischemic cardiac arrest with successful resuscitation, 5) no acute coronary event or 6) an unclassifiable fatal case. The categories 1) to 3) were combined for the present analysis to denote AMI.

The cases were defined so that they had either a confirmed definite or probable AMI or typical prolonged chest pain and a family history of AMI (at least one affected family member, either a sibling or a parent). These characteristics were determined to increase the likelihood that the coronary disease in the case subjects was caused by genes and not by non-genetic factors. Analogically, the controls did not have family history of AMI in either their parents of siblings.

An identical number of healthy control subjects were selected from the same KIHD cohort as the cases. They had no family history of CHD in parents or siblings. To minimize the control-dilution bias (controls developing AMI later), CHD-free controls were selected from very healthy persons. The controls were free of CHD, assessed broadly. The controls for GWS had neither diagnosed CHD, symptoms or signs of CHD, nitroglycerin medication, ischaemic ECG findings in maximal exercise test, type 2 diabetes nor moderate-to-severe hypertension. The proportion of males was equal among both the cases and the controls. To control for confounding, the controls were matched according to gender, smoking status and the municipality of residence. In this founder-population-based familial case-control design, the number of both the cases and the controls used in the initial GWS was 125 (125+125=250). Selected characteristics of the cases and controls are shown in table 2. As the controls had been matched, the age and the number of cigarettes smoked daily were similar in both groups.

Forty of the 125 patients who experienced AMI during the follow-up also died of CHD during the follow-up up to the end of 2003. Coronary death was defined as death for which the underlying cause was determined to be ICD-9 code 410-414 or ICD-10 code I20-I25. In the statistical analysis, the 40 case subjects who died of CHD were compared with all other 206 male subjects who did not die of CHD during the follow-up. Eighty-five of these had experienced a non-fatal AMI during the follow-up and 121 men remained free of CHD during the follow-up. The main statistical analyses were repeated after exclusion of the 85 men who experienced a non-fatal AMI. As the findings were virtually identical, the results of those analyses will not be presented.

TABLE 2
Selected characteristics of the cases and controls

| | Cases (n = 40) | | | Controls (n = 206) | | |
|---|---|---|---|---|---|---|
| | Mean | Min | Max | Mean | Min | Max |
| Age (years) | 55.8 | 42.5 | 61.0 | 53.8 | 42.1 | 61.3 |
| Cigarettes/day | 8.6 | 0 | 40 | 5.6 | 0 | 40 |
| S-Cholesterol | 6.42 | 3.83 | 8.72 | 5.98 | 3.05 | 9.09 |
| (mmol/L) | | | | | | |
| S-HDL-Chol | 1.30 | 0.84 | 2.77 | 1.29 | 0.76 | 2.38 |
| (mmol/L) | | | | | | |
| P-fibrinogen (g/L) | 3.17 | 1.77 | 4.14 | 3.02 | 2.14 | 5.11 |
| B-Glucose | 5.12 | 3.6 | 12.6 | 4.68 | 3.3 | 12.3 |
| (mmol/L) | | | | | | |
| S-Insulin (U/L) | 13.8 | 4.3 | 35.9 | 10.7 | 1.7 | 59.6 |

| | | |
|---|---|---|
| Prevalent CHD at | 57.5% | 13.6% |
| baseline | | |

The table 2 summarizes selected characteristics of the cases and controls. Age and tobacco smoking were recorded on a self-administered questionnaire checked by an interviewer. Fasting blood glucose was measured using a glucose dehydrogenase method after precipitation of proteins by trichloroacetic acid. Serum insulin was determined with a Novo Biolabs radioimmunoassay kit (Novo Nordisk). HDL fractions were separated from fresh serum by combined ultracentrifugation and precipitation. The cholesterol contents of lipoprotein fractions and serum triglycerides were measured enzymatically. Fibrinogen was measured based on the clotting of diluted plasma with excess thrombin.

Hypertension was defined as either systolic blood pressure (SBP)≧165 mmHg or diastolic BP (DBP)≧95 mmHg or antihypertensive treatment. Both blood pressures were measured in the morning by a nurse with a random-zero mercury sphygmomanometer. The measuring protocol included three measurements in supine, one in standing and two in sitting position with 5-minutes intervals. The mean of all six measurements were used as SBP and DBP.

Family history of CHD was defined positive if the subject's mother, father or a sibling had a history of either AMI or angina pectoris. Family histories of cerebrovascular stroke and diabetes were defined similarly. Adulthood socioeconomical status (SES) is an index comprised of measures of education, occupation, income and material living conditions. The scale is inverse, low score corresponding to high SES. These data have been collected by a self administered questionnaire.

Serum ferritin was assessed with a commercial double antibody radioimmunoassay (Amersham International, Amersham, UK). Lipoproteins, including high density lipoprotein (HDL) and low density lipoprotein (LDL), were separated from fresh serum samples by ultracentrifugation followed by direct very low density lipoprotein (VLDL) removal and LDL precipitation (Salonen et al 1991). Cholesterol concentration was then determined enzymically. Serum C-reactive protein was measured by a commercial high-sensitive immunometric assay (Immulite High Sensitivity CR Assay, DPC, Los Angeles).

Prevalent CHD was defined positive if the subject reported a history of AMI or angina pectoris or a coronary by-pass surgery, used sublingual nitroglycerin tablets or had a angina pectoris on effort according to the London School of Hygiene Chest Pain Questionnaire. As there was a large difference in the proportion of subjects with prevalent CHD between those who died of CHD (57.5%) and those who did not (13.6%), all analyses comparing the 40 cases who died and 206 controls who did not die of CHD also concern the comparison between those who had CHD and those who did not have CHD. Because of the similarity of the results from this comparison, those findings are not presented separately.

Genomic DNA Isolation and Quality Testing

High molecular weight genomic DNA samples were extracted from frozen venous whole blood using standard methods and dissolved in standard TE buffer. The quantity and purity of each DNA sample was evaluated by measuring the absorbance at 260 and 280 nm and integrity of isolated DNA samples was evaluated with 0.9% agarose gel electrophoresis and Ethidiumbromide staining. A sample was qualified for genome wide scan (GWS) analysis if A260/A280 ratio was ≧1.7 and average size of isolated DNA was over 20 kb in agarose gel electrophoresis. Before GWS analysis samples were diluted to concentration of 50 ng/μl in reduced EDTA TE buffer (TEKnova).

Genome-Wide Scan

Genotyping of SNP markers was performed by using the technology access version of Affymetrix GeneChip® human mapping 100 k system. The assay consisted of two arrays, Xba and Hind, which were used to genotype over 126,000 SNP markers from each DNA sample. The assays were performed according to the instructions provided by the manufacturer. A total of 250 ng of genomic DNA was used for each individual assay. DNA sample was digested with either Xba I or Hind III enzyme (New England Biolabs, NEB) in the mixture of NE Buffer 2 (1×; NEB), bovine serum albumin (1×; NEB), and either Xba I or Hind III (0.5 U/μl; NEB) for 2 h at +37° C. followed by enzyme inactivation for 20 min at +70° C. Xba I or Hind III adapters were then ligated to the digested DNA samples by adding Xba or Hind III adapter (0.25 μM, Affymetrix), T4 DNA ligase buffer (1×; NEB), and T4 DNA ligase (250 U; NEB). Ligation reactions were allowed to proceed for 2 h at +16° C. followed by 20 min incubation at +70° C. Each ligated DNA sample was diluted with 75 μL of molecular biology-grade water (BioWhittaker Molecular Applications/Cambrex).

Diluted ligated DNA samples were subjected to four identical 100 μl volume polymerase chain reactions (PCR) by implementing a 10 μl aliquot of DNA sample with Pfx Amplification Buffer (1×; Invitrogen), PCR Enhancer (1×; Invitrogen), MgSO₄ (1 mM; Invitrogen), dNTP (300 μM each; Takara), PCR primer (1 μM; Affymetrix), and Pfx Polymerase (0.05 U/μl; Invitrogen). The PCR was allowed to proceed for 3 min at +94° C., followed by 30 cycles of 15 sec at +94° C., 30 sec at +60° C., 60 sec at +68° C., and finally for the final extension for 7 min at +68° C. The performance of the PCR was checked by standard 2% agarose gel electrophoresis in 1×TBE buffer for 1 h at 120V.

PCR products were purified according to Affymetrix manual using MinElute 96 UF PCR Purification kit (Qiagen) by combining all four PCR products of an individual sample into same purification reaction. The purified PCR products were eluted with 40 μl of EB buffer (Qiagen), and the yields of the products were measured at the absorbance 260 nm. A total of 40 μg of each PCR product was then subjected to fragmentation reaction consisting of 0.2 U/μl fragmentation reagent (Affymetrix) in 1× Fragmentation Buffer. Fragmentation reaction was allowed to proceed for 35 min at +37° C. followed by 15 min incubation at +95° C. for enzyme inactivation. Completeness of fragmentation was checked by running an aliquot of each fragmented PCR product in 4% agarose 1×TBE (BMA Reliant precast) for 30-45 min at 120V.

Fragmented PCR products were then labeled using 1× Terminal Deoxinucleotidyl Transferase (TdT) buffer (Affymetrix), GeneChip DNA Labeling Reagent (0.214 mM; Affymetrix), and TdT (1.5 U/μl; Affymetrix) for 2 h at +37° C. followed by 15 min at +95° C. Labeled DNA samples were combined with hybridization buffer consisting of 0.056 M MES solution (Sigma), 5% DMSO (Sigma), 2.5× Denhardt's solution (Sigma), 5.77 mM EDTA (Ambion), 0.115 mg/ml Herring Sperm DNA (Promega), 1× Oligonucleotide Control reagent (Affymetrix), 11.5 μg/ml Human Cot-1 (Invitrogen), 0.0115% Tween-20 (Pierce), and 2.69 M Tetramethyl Ammonium Chloride (Sigma). DNA-hybridization buffer mix was denatured for 10 min at +95° C., cooled on ice for 10 sec and incubated for 2 min at +48° C. prior to hybridization onto corresponding Xba or Hind GeneChip® array. Hybridization was completed at +48° C. for 16-18 h at 60 rpm in an Affymetrix GeneChip Hybridization Oven. Following hybridization, the arrays were stained and washed in GeneChip Fluidics Station 450 according to fluidics station protocol Mapping10Kv1_{—}450 as recommended by the manufacturer. Arrays were scanned with GeneChip 3000 Scanner and the genotype calls for each of the SNP markers on the array were generated using Affymetrix Genotyping Tools (GTT) software. The confidence score in SNP calling algorithm was adjusted to 0.20.

Initial SNP Selection for Statistical Analysis

Prior to the statistical analysis, SNP quality was assessed on the basis of three values: the call rate (CR), minor allele frequency (MAF), and Hardy-Weinberg equilibrium (H-W). The CR is the proportion of samples with successful genotyping result. It does not take into account whether the genotypes are correct or not. The call rate was calculated as: CR=number of samples with successful genotype call/total number of samples. The MAF is the frequency of the allele that is less frequent in the study sample. MAF was calculated as: MAF=min(p, q), where p is frequency of the SNP allele ‘A’ and q is frequency of the SNP allele ‘B’; p=(number of samples with “AA”-genotype+0.5*number of samples with “AB”-genotype)/total number of samples with successful genotype call; q=1−p. SNPs that are homozygous (MAF=0) can not be used in genetic analysis and were thus discarded. H-W equilibrium is tested for controls. The test is based on the standard Chi-square test of goodness of fit. The observed genotype distribution is compared with the expected genotype distribution under H-W equilibrium. For two alleles this distribution is p2, 2pq, and q² for genotypes ‘AA’, ‘AB’ and ‘BB’, respectively. If the SNP is not in H-W equilibrium it can be due to genotyping error or some unknown population dynamics (e.g. random drift, selection).

Only the SNPs that had CR>50%, MAF>1%, and were in H-W equilibrium (Chi-square test statistic<23.93) were used in the statistical analysis. A total of 107,895 SNPs fulfilled the above criteria and were included in the statistical analysis.

Statistical Methods

Single SNP Analysis

Differences in allele distributions between cases and controls were screened for all 107,895 SNPs. The screening was carried out by using the standard Chi-square independence test with 1 df (allele distribution, 2×2 table). SNPs that gave P-value less than 0.005 (Chi-square distribution with 1 df of 7.88 or more) were considered as statistically significant and selected for further analysis. There were 757 SNPs that fulfilled this criterium.

Haplotype Analysis

The data set was analyzed with a haplotype pattern mining algorithm with HPM software (Toivonen H T et al, 2000). For HPM software genotypes must have phase known i.e. to determine which alleles are coming from the mother and which from the father. Without family data phases must be estimated based on population data. We used HaploRec-program (Eronen L et al, 2004) to estimate the phases. HPM is very fast and can handle a large number of SNPs in a single run.

Several parameters can be modified in the HPM programs including the Chi-square threshold value (−x), the maximum haplotype pattern length (−l), the maximum number of wildcards that can be included in a haplotype pattern (−w), and the number of permutation test in order to estimate the P-value (−p). Wildcards allow gaps in haplotypes. HPM was run with the following parameter settings: haplotype analysis with 5 SNPs (−x9 −l5 −w1 −p10000). Based on 10,000 replicates (−p100000) in the HPM analyses 949 SNPs were significant at P-value less than 0.005.

Definition of Terms Used in the Haplotype Analysis Results.

The term “haplotype genomic region” or “haplotype region” refers to a genomic region that has been found significant in the haplotype analysis (HPM or similar statistical method/program). The haplotype region is defined as 100 Kbp up/downstream from the physical position the first/last SNP that was included in the statistical analysis (haplotype analysis) and was found statistically significant. This region is given in based pairs based on the given genome build e.g. SNP physical position (basepair position) according to NCBI Human Genome Build 35.

The term “haplotype”, as described herein, refers to any combination of alleles e.g. A T C T A that is found in the given genetic markers e.g rs2301081 (SEQ ID NO:832), rs1966580 (SEQ ID NO:718), rs2301086 (SEQ ID NO:833), rs1346572 (SEQ ID NO:497), and rs10514263 (SEQ ID NO:355). A defined haplotype gives the name of the genetic markers (dbSNP rs-id for the SNPs) and the alleles. As it is recognized by those skilled in the art the same haplotype can be described differently by determining alleles from different strands e.g. the haplotype rs2301081 (SEQ ID NO:832), rs1966580 (SEQ ID NO:718), rs2301086 (SEQ ID NO:833), rs1346572 (SEQ ID NO:497), rs10514263 (SEQ ID NO:355) (A T C T A) is the same as haplotype rs2301081 (SEQ ID NO:832), rs1966580 (SEQ ID NO:718), rs2301086 (SEQ ID NO:833), rs1346572 (SEQ ID NO:497), rs10514263 (SEQ ID NO:355) (T A G A T) in which the alleles are determined from the other strand or haplotype rs2301081 (SEQ ID NO:832), rs1966580 (SEQ ID NO:718), rs2301086 (SEQ ID NO:833), rs1346572 (SEQ ID NO:497), rs10514263 (SEQ ID NO:355) (T T C T A), in which the first allele is determined from the other strand.

The haplotypes described herein, e.g., having markers such as those shown in tables 4, 5, 7 and 8, are found more frequently in individuals with CHD death than in individuals without CHD death. Therefore, these haplotypes have predictive value for detecting CHD death or a susceptibility to CHD death in an individual. Therefore, detecting haplotypes can be accomplished by methods known in the art for detecting sequences at polymorphic sites.

It is understood that the CHD death associated at-risk alleles and at-risk haplotypes described in this invention may be associated with other “polymorphic sites” located in CHD death associated genes of this invention. These other CHD death associated polymorphic sites may be either equally useful as genetic markers or even more useful as causative variations explaining the observed association of at-risk alleles and at-risk haplotypes of this invention to CHD death.

Multivariate Modeling

Of the 757 SNPs from the screening of individual markers and 949 SNPs from the haplotype pattern analyses, there were 1400 SNPs (306 SNPs were the same in both screens). These were recoded as dummy variables in two ways: a) Homozygote of the minor allele coded as 1, otherwise 0, and b) Carrier of the minor allele coded as 1, otherwise 0. A multivariate binary logistic function regression analysis was used to: a) Find the SNPs that were most predictive of CHD death and b) Construct a multivariate model that predicted CHD death the strongest.

A forward step-up model construction was used with p-value to enter of 0.01 and p-value to exclude from the model of 0.02. The predictivity of the models was estimated by two methods: the Nagelkerke R square and the reclassification of the subjects to cases and controls on the basis of the logistic model contructed. The predicted probability used as cut-off was 0.5. A data reduction analysis was carried out by step-down and step-up logistic modeling. The statistical software used was SPSS for Windows, version 11.5.

Results

In table 3 (on CD) are summarized the characteristics of the SNP markers with the strongest association with CHD death in the individual marker analysis. SNP identification number according to NCBI dbSNP database build 124. SNP physical position according to NCBI Human Genome Build 35. Gene locus as reported by NCBI dbSNP database build 124. SNP flanking sequences were from the Affymetrix “csv” commercial access Human Mapping 100K array annotation files.

In table 4 (on CD) are summarized the characteristics of the haplotype genomic regions with the strongest association with CHD death in the HPM analysis with 5 SNPs. SNP identification number according to NCBI dbSNP database build 124. SNP physical position according to NCBI Human Genome Build 35. Associated genes are those genes positioned within 100 Kbp up/downstream from the physical position of the SNPs bordering the haplotype genomic region found using NCBI MapViewer, based on NCBI Human Genome Build 35. SNP flanking sequence provided by Affymetrix “csv” commercial access Human Mapping 100K array annotation files.

In table 5 (on CD) are listed haplotype blocks with the strongest association with CHD death based on HaploRec+HPM analysis. SNP identification number according to NCBI dbSNP database build 124.

In table 6 (on CD) are listed all genes found associated with CHD death according to point wise or haplotype analyses. Gene name according to HUGO Gene Nomenclature Committee (HGNC).

In table 7 (on CD) are listed the SNP-markers and haplotypes that best predicted risk CHD death in a multivariate logistic model. The model was constructed by a step-up procedure, using P=0.01 for entry criterium and P=0.02 as exclusion criterium. SNP identification number according to NCBI dbSNP database build 124. The model includes five haplotypes and two individual SNP markers. The 7-variable model predicts 93.9% of CHD deaths correctly.

In table 8 (on CD) are listed the SNP-markers and haplotypes that best predicted risk CHD death in a multivariate logistic model. Haplotypes were included as dummy variables (coded as 0, if no haplotype, 1 if one haplotype and 2 if two haplotypes). SNP identification number according to NCBI dbSNP database build 124. The model includes five haplotypes and two individual SNP markers. The 11-variable model predicts 94.3% of CHD deaths correctly.

In table 9 (on CD) are listed the SNP-markers, haplotypes and phenotypic measurements that best predicted risk CHD death in a multivariate logistic model. SNP identification number according to NCBI dbSNP database build 124. The model includes five haplotypes, two individual SNP markers and two phenotypic variables (age and plasma insulin concentration). The 13-variable model predicts 95.5% of CHD deaths correctly.

### IMPLICATIONS AND CONCLUSIONS

We have found associations between 1503 SNP markers and the risk of CHD death in a population-based prospective nested set of familial cases and extremely healthy controls. These markers were also associated with chronic prevalent CHD at baseline. Of these, 757 were identified in the analysis of individual SNPs and 1054 in haplotype sharing analysis. Of the 1503 markers, 308 predicted CHD death in both types of statistical analysis. We further identified several sets of SNP markers and haplotypes, which predict in a multivariate logistic model virtually fully the development of CHD death.

The results of the pointwise and haplotype analyses identified a total of 740 genes associated with CHD death, of which 299 genes had at least one of the 1503 SNP markers physically linked to the gene.

Thus, we have discovered a total of 740 CHD genes, in which any genetic markers can be used to predict CHD, and thus these markers can be used as part of molecular diagnostic tests of CHD predisposition. In addition, we have disclosed a set of 1503 SNP markers which are predictive of CHD death. The markers can also be used as part of pharmacogenetic tests which predict the efficacy and adverse reactions of anti-coronary agents and compounds. The genes discovered are also targets to new therapies of CHD, such as drugs. Other therapies are molecular, including gene transfer. The new genes can also be used to develop and produce new transgenic animals and in Vitro models for studies of anti-coronary agents and compounds.

While this invention has been particularly shown and described with reference to preferred embodiments thereof, it will be understood by those skilled in the art that various changes in form and details may be made therein without departing from the spirit and scope of the invention as defined by the appended claims.

### REFERENCES

American Heart Association. Heart Disease and Stroke Statistics—2004 Update. http://www.americanheart.org/downloadable/heart/1079736729696HDSStats2004Up dateREV3-19-04.pdf (Accessed 14.07.04).
Ardissino D, Mannucci P M, Merlini P A, Duca F, Fetiveau R, Tagliabue L, Tubaro M, Galvani M, Ottani F, Ferrario M, Corral J, Margaglione M. 1999. Prothrombotic genetic risk factors in young survivors of myocardial infarction. Blood. 94:46-51.
Ausubel F M, Brent R, Kingston R E, Moore D D, Seidman J G, Struhl K. 2003. Current Protocols in Molecular Biology. NY:John Wiley & Sons.
Baroni M G, Berni A, Romeo S, Arca M, Tesorio T, Sorropago G, Di Mario U, Galton D J. 2003. Genetic study of common variants at the Apo E, Apo AI, Apo CIII, Apo B, lipoprotein lipase (LPL) and hepatic lipase (LIPC) genes and coronary artery disease (CAD): variation in LIPC gene associates with clinical outcomes in patients with established CAD. BMC Med Genet. 4:8.
Barrett-Connor E, Khaw K. 1984. Family history of heart attack as an independent predictor of death due to cardiovascular disease. Circulation 69: 1065-9.
Beyzade S, Zhang S, Wong Y K, Day I N, Eriksson P, Ye S. 2003. Influences of matrix metalloproteinase-3 gene variation on extent of coronary atherosclerosis and risk of myocardial infarction. J Am Coll Cardiol. 41:2130-7.
Bierer B, Coligan J E, Margulies D H, Shevach E M, Strober W. 2002. Current Protocols in Immunology. NY:John Wiley & Sons.
Boer J M, Feskens E J, Verschuren W M, Seidell J C, Kromhout D. 1999. The joint impact of family history of myocardial infarction and other risk factors on 12-year coronary heart disease mortality. Epidemiology 10:767-70.
Broeckel U, Hengstenberg C, Mayer B, Holmer S, Martin L J, Comuzzie A G, Blangero J, Nurnberg P, Reis A, Riegger G A, Jacob H J, Schunkert H. 2002. A comprehensive linkage analysis for myocardial infarction and its related risk factors. Nat Genet. 30:210-4.
Brscic E, Bergerone S, Gagnor A, Colajanni E, Matullo G, Scaglione L, Cassader M, Gaschino G, Di Leo M, Brusca A, Pagano G F, Piazza A, Trevi G P. 2000. Acute myocardial infarction in young adults: prognostic role of angiotensin-converting enzyme, angiotensin II type I receptor, apolipoprotein E, endothelial constitutive nitric oxide synthase, and glycoprotein IIIa genetic polymorphisms at medium-term follow-up. Am Heart J. 139:979-84.
Burzotta F, Paciaroni K, De Stefano V, Crea F, Maseri A, Leone G, Andreotti F. 2004. G20210A prothrombin gene polymorphism and coronary ischaemic syndromes: a phenotype-specific meta-analysis of 12 034 subjects. Heart. 90:82-6.
Chao T H, Li Y H, Chen J H, Wu H L, Shi G Y, Tsai W C, Chen P S, Liu P Y. 2004. Relation of thrombomodulin gene polymorphisms to acute myocardial infarction in patients <or =50 years of age. Am J Cardiol. 93:204-7.
Chen X, Levine L, Kwok P Y. 1999. Fluorescence polarization in homogeneous nucleic acid analysis. Genome Res. 9:492-8.
Chiodini B D, Barlera S, Franzosi M G, Beceiro V L, Introna M, Tognoni G. 2003. APO B gene polymorphisms and coronary artery disease: a meta-analysis. Atherosclerosis. 167:355-66.
Chiodini B D, Lewis C M. 2003. Meta-analysis of 4 coronary heart disease genome-wide linkage studies confirms a susceptibility locus on chromosome 3q. Arterioscler Thromb Vasc Biol. 23:1863-8.
Chisolm G M, Steinberg D. 2000. The oxidative modification hypothesis of atherogenesis: an overview. Free Radic Biol Med 28: 1815-26.
Church G M, Gilbert W. 1984. Genomic sequencing. Proc Natl Acad Sci USA. 81:1991-5.
Cine N, Hatemi A C, Erginel-Unaltuna N. 2002. Association of a polymorphism of the ecNOS gene with myocardial infarction in a subgroup of Turkish MI patients. Clin Genet. 61:66-70.
Civeira F. 2004. International Panel on Management of Familial Hypercholesterolemia. Guidelines for the diagnosis and management of heterozygous familial hypercholesterolemia. Atherosclerosis. 173:55-68.
Colditz G A, Rimm E B, Giovannucci E, Stampfer M J, Rosner B, Willett W C. 1991. A prospective study of parental history of myocardial infarction and coronary artery disease in men. Am J Cardiol 67: 933-8.
Colditz G A, Stampfer M J, Willett W C, Rosner B, Speizer F E, Hennekens C H. 1986. A prospective study of parental history of myocardial infarction and coronary heart disease in women. Am J Epidemiol 123: 48-58.
Cole S P, Campling B G, Atlaw T, Kozbor D, Roder J C. 1984. Human monoclonal antibodies. Mol Cell Biochem. 62:109-20.
Davies M J. 1990. A macro and micro view of coronary vascular insult in ischemic heart disease. Circulation 82(3 Suppl): 1138-46.
Deiman B, van Aarle P, Sillekens P. 2002. Characteristics and applications of nucleic acid sequence-based amplification (NASBA). Mol Biotechnol. 20:163-79.
Dianzani I, Forrest S M, Camaschella C, Gottardi E, Cotton R G. 1991. Heterozygotes and homozygotes: discrimination by chemical cleavage of mismatch. Am J Hum Genet. 48:423-4.
Eckert K A, Kunkel T A. 1991. DNA polymerase fidelity and the polymerase chain reaction. PCR Methods Appl. 1:17-24.
Eichner J E, Kuller L H, Orchard T J, Grandits G A, McCallum L M, Ferrell R E, Neaton J D. 1993. Relation of apolipoprotein E phenotype to myocardial infarction and mortality from coronary artery disease. Am J Cardiol. 71:160-5.
Eronen L, Geerts F, Toivonen H. 2004. A Markov chain approach to reconstruction of long haplotypes. Pac Symp Biocomput.: 104-15.
Fatkin D, Graham R M. 2002. Molecular mechanisms of inherited cardiomyopathies. Physiol Rev. 82:945-80.
Fodor S P, Read J L, Pirrung M C, Stryer L, Lu A T, Solas D. 1991. Light-directed, spatially addressable parallel chemical synthesis. Science. 251:767-73.
Fox C S, Cupples L A, Chazaro I, Polak J F, Wolf P A, D'Agostino R B, Ordovas J M, O'Donnell C J. 2004. Genomewide linkage analysis for internal carotid artery intimal medial thickness: evidence for linkage to chromosome 12. Am J Hum Genet. 74:253-61.
Francke S, Manraj M, Lacquemant C, Lecoeur C, Lepretre F, Passa P, Hebe A, Corset L, Yan S L, Lahmidi S, Jankee S, Gunness T K, Ramjuttun U S, Balgobin V, Dina C, Froguel P. 2001. A genome-wide scan for coronary heart disease suggests in Indo-Mauritians a susceptibility locus on chromosome 16p 13 and replicates linkage with the metabolic syndrome on 3q27. Hum Mol Genet. 10:2751-65.
Franz W M, Muller O J, Katus H A. 2001. Cardiomyopathies: from genetics to the prospect of treatment. Lancet. 358:1627-37.
Fu L, Jin H, Song K, Zhang C, Shen J, Huang Y. 2001. Relationship between gene polymorphism of the PAI-I promoter and myocardial infarction. Chin Med J (Engl). 114:266-9.
Gardemann A, Lohre J, Katz N, Tillmanns H, Hehrlein F W, Haberbosch W. 1999. The 4G4G genotype of the plasminogen activator inhibitor 4G/5G gene polymorphism is associated with coronary atherosclerosis in patients at high risk for this disease. Thromb Haemost. 82:1121-6.
Geever R F, Wilson L B, Nallaseth F S, Milner P F, Bittner M, Wilson J T. Direct identification of sickle cell anemia by blot hybridization. 1981. Proc Natl Acad Sci USA. 78:5081-5.
Georges J L, Loukaci V, Poirier 0, Evans A, Luc G, Arveiler D, Ruidavets J B, Cambien F, Tiret L. 2001. Interleukin-6 gene polymorphisms and susceptibility to myocardial infarction: the ECTIM study. Etude Cas-Temoin de l'Infarctus du Myocarde. J Mol Med. 79:300-5.
Gibbs R A, Nguyen P N, Caskey C T. 1989. Detection of single DNA base differences by competitive oligonucleotide priming. Nucleic Acids Res. 17:2437-48.
Gold B. 2003. Origin and utility of the reverse dot-blot. Expert Rev Mol Diagn. 3:143-52.
Gomes A V, Potter J D. Molecular and cellular aspects of troponin cardiomyopathies. Ann N Y Acad Sci 2004;1015:214-24.
Griffiths A D, Malmqvist M, Marks J D, Bye J M, Embleton M J, McCafferty J, Baier M,
Holliger K P, Gorick B D, Hughes-Jones N C, et al. 1993. Human anti-self antibodies with high specificity from phage display libraries. EMBO J. 12:725-34.
Guatelli J C, Whitfield K M, Kwoh D Y, Barringer K J, Richman D D, Gingeras T R. 1990. Isothermal, in vitro amplification of nucleic acids by a multienzyme reaction modeled after retroviral replication. Proc Natl Acad Sci USA. 87:1874-8.
Harrap S B, Zammit K S, Wong Z Y, Williams F M, Bahlo M, Tonkin A M, Anderson S T. 2002. Genome-wide linkage analysis of the acute coronary syndrome suggests a locus on chromosome 2. Arterioscler Thromb Vasc Biol. 22:874-8.
Hawe E, Talmud P J, Miller G J, Humphries S E; Second Northwick Park Heart Study. 2003. Family history is a coronary heart disease risk factor in the Second Northwick Park Heart Study. Ann Hum Genet. 67:97-106.
Hay B N, Sorge J A, Shopes B. 1992. Bacteriophage cloning and Escherichia coli expression of a human IgM Fab. Hum Antibodies Hybridomas. 3:81-5.
Hayashi N, Kipriyanov S, Fuchs P, Welschof M, Dorsam H, Little M. 1995. A single expression system for the display, purification and conjugation of single-chain antibodies. Gene. 160:129-30.
Helene C, Thuong N T, Harel-Bellan A. 1992. Control of gene expression by triple helix-forming oligonucleotides. The antigene strategy. Ann N Y Acad. Sci. 660:27-36.
Helene C. 1991. The anti-gene strategy: control of gene expression by triplex-forming-oligonucleotides. Anticancer Drug Des. 6:569-84.
Heller R F, Kelson M C. 1983. Family history in “low risk” men with coronary heart disease. J Epidemiol Community Health 37: 29-31.
Hering S, Karawajew L, Pasternak G. 1988. Raji-K562 hybrids and their use for trioma production. Biomed Biochim Acta. 47:211-6.
Hibi K, Ishigami T, Tamura K, Mizushima S, Nyui N, Fujita T, Ochiai H, Kosuge M, Watanabe Y, Yoshii Y, Kihara M, Kimura K, Ishii M, Umemura S. 1998. Endothelial nitric oxide synthase gene polymorphism and acute myocardial infarction. Hypertension. 32:521-6.
Hingorani A D, Liang C F, Fatibene J, Lyon A, Monteith S, Parsons A, Haydock S, Hopper R V, Stephens N G, O'Shaughnessy K M, Brown M J. 1999. A common variant of the endothelial nitric oxide synthase (Glu298-->Asp) is a major risk factor for coronary artery disease in the UK. Circulation. 100:1515-20.
Holm J, Hillarp A, Zoller B, Erhardt L, Berntorp E, Dahlback B. 1999. Factor V Q506 (resistance to activated protein C) and prognosis after acute coronary syndrome. Thromb Haemost. 81:857-60.
Hopkins P N, Williams R R, Kuida H, Stults B M, Hunt S C, Barlow G K, Ash K O. 1988. Family history as an independent risk factor for incident coronary artery disease in a high-risk cohort in Utah. Am J Cardiol 62: 703-7.
Huber M, Mundlein A, Dornstauder E, Schneeberger C, Tempfer C B, Mueller M W, Schmidt W M. 2002. Accessing single nucleotide polymorphisms in genomic DNA by direct multiplex polymerase chain reaction amplification on oligonucleotide microarrays. Anal Biochem. 303:25-33.
Humphries S E, Luong L A, Ogg M S, Hawe E, Miller G J. 2001. The interleukin-6-174 G/C promoter polymorphism is associated with risk of coronary heart disease and systolic blood pressure in healthy men. Eur Heart J. 22:2243-52.
Humphries S E, Martin S, Cooper J, Miller G. 2002. Interaction between smoking and the stromelysin-1 (MMP3) gene 5A/6A promoter polymorphism and risk of coronary heart disease in healthy men. Ann Hum Genet. 66:343-52.
Humphries S E, Talmud P J, Hawe E, Bolla M, Day I N, Miller G J. 2001. Apolipoprotein E4 and coronary heart disease in middle-aged men who smoke: a prospective study. Lancet. 358:115-9.
Humphries S E, Talmud P J, Hawe E, Bolla M, Day I N, Miller G J. 2001. Apolipoprotein E4 and coronary heart disease in middle-aged men who smoke: a prospective study. Lancet. 358:115-9.
Huse W D, Sastry L, Iverson S A, Kang A S, Alting-Mees M, Burton D R, Benkovic S J, Lerner R A. 1989. Generation of a large combinatorial library of the immunoglobulin repertoire in phage lambda. Science. 246:1275-81.
Inbal A, Freimark D, Modan B, Chetrit A, Matetzky S, Rosenberg N, Dardik R, Baron Z, Seligsohn U. 1999. Synergistic effects of prothrombotic polymorphisms and atherogenic factors on the risk of myocardial infarction in young males. Blood. 93:2186-90.
Iwahana H, Yoshimoto K, Itakara M. 1992. Detection of point mutations by SSCP of PCR-amplified DNA after endonuclease digestion. Biotechniques. 12:64-66.
Jaeger L B, Banks W A. 2004. Antisense therapeutics and the treatment of CNS disease. Front Biosci. 9:1720-7.
Jiricny J, Su S S, Wood S G, Modrich P. 1988. Mismatch-containing oligonucleotide duplexes bound by the E. coli mutS-encoded protein. Nucleic Acids Res. 16:7843-53.
Jousilahti P, Puska P, Vartiainen E, Pekkanen J, Tuomilehto J. 1996. Parental history of premature coronary heart disease: an independent risk factor of myocardial infarction. J Clin Epidemiol 49: 497-503.
Kim R J, Becker R C. 2003. Association between factor V Leiden, prothrombin G20210A, and methylenetetrahydrofolate reductase C677T mutations and events of the arterial circulatory system: a meta-analysis of published studies. Am Heart J. 146:948-57.
Kohler G, Milstein C. 1975. Continuous cultures of fused cells secreting antibody of predefined specificity. Nature 256:495-497.
Kozbor D, Lagarde A E, Roder J C. 1982. Human hybridomas constructed with antigen-specific Epstein-Barr virus-transformed cell lines. Proc Natl Acad Sci USA. 79:6651-5.
Kumar P, Luthra K, Dwivedi M, Behl V K, Pandey R M, Misra A. 2003. Apolipoprotein E gene polymorphisms in patients with premature myocardial infarction: a case-controlled study in Asian Indians in North India. Ann Clin Biochem. 40:382-7.
Kwoh D Y, Davis G R, Whitfield K M, Chappelle H L, DiMichele L J, Gingeras T R. 1989. Transcription-based amplification system and detection of amplified human immunodeficiency virus type 1 with a bead-based sandwich hybridization format. Proc Natl Acad Sci USA. 86:1173-7.
Kwok P-Y. 2001. Methods for genotyping single nucleotide polymorphisms. Ann Rev Genomics Hum Genet. 2:235-258.
Landegren U, Kaiser R, Sanders J, Hood L. 1988. A ligase-mediated gene detection technique. Science. 241:1077-80.
Lemaitre M, Bayard B, Lebleu B. 1987. Specific antiviral activity of a poly(L-lysine)-conjugated oligodeoxyribonucleotide sequence complementary to vesicular stomatitis virus N protein mRNA initiation site. Proc Natl Acad Sci USA. 84:648-52.
Letsinger R L, Zhang G R, Sun D K, Ikeuchi T, Sarin P S. 1989. Cholesteryl-conjugated oligonucleotides: synthesis, properties, and activity as inhibitors of replication of human immunodeficiency virus in cell culture. Proc Natl Acad Sci USA. 86:6553-6.
Li R, Bensen J T, Hutchinson R G, Province M A, Hertz-Picciotto I, Sprafka J M, Tyroler H A. 2000. Family risk score of coronary heart disease (CHD) as a predictor of CHD: the Atherosclerosis Risk in Communities (ARIC) study and the NHLBI family heart study. Genet Epidemiol 18: 236-50.
Li Y H, Chen J H, Tsai W C, Chao T H, Guo H R, Tsai L M, Wu H L, Shi G Y. 2002. Synergistic effect of thrombomodulin promoter −33G/A polymorphism and smoking on the onset of acute myocardial infarction. Thromb Haemost. 87:86-91.
Libby P. 1995. Molecular bases of the acute coronary syndromes. Circulation 91: 2844-50.
Marenberg M E, Risch N, Berkman L F, Floderus B, de Faire U. 1994. Genetic susceptibility to death from coronary heart disease in a study of twins. N Engl J. Med. 330:1041-6.
Meade H, Gates L, Lacy E, Lonberg N. 1990. Bovine alpha S1-casein gene sequences direct high level expression of active human urokinase in mouse milk. Biotechnology (N Y). 8:443-6.
Mikkelsson J, Perola M, Wartiovaara U, Peltonen L, Palotie A, Penttila A, Karhunen P J. 2000. Plasminogen activator inhibitor-1 (PAI-1) 4G/5G polymorphism, coronary thrombosis, and myocardial infarction in middle-aged Finnish men who died suddenly. Thromb Haemost. 84:78-82.
Murray C J L, Lopez A D. 1997. Global mortality, disability, and the contribution of risk factors: Global Burden of Disease Study. Lancet 349:1436-42.
Mustafina O E, Shagisultanova E I, Tuktarova I A, Khusnutdinova E K. 2002. Polymorphism of the apolipoprotein E gene and the risk of myocardial infarction. Mol Biol (Mosk). 36:978-84.
Myers R H, Kiely D K, Cupples L A, Kannel W B. 1990. Parental history is an independent risk factor for coronary artery disease: the Framingham Study. Am Heart J 120: 963-9.
Myers R M, Fischer S G, Lerman L S, Maniatis T. 1985. Nearly all single base substitutions in DNA fragments joined to a GC-clamp can be detected by denaturing gradient gel electrophoresis. Nucleic Acids Res. 13:3131-45.
Myers R M, Larin Z, Maniatis T. 1985. Detection of single base substitutions by ribonuclease cleavage at mismatches in RNA: DNA duplexes. Science. 230:1242-6.
Nakai K, Fusazaki T, Zhang T, Shiroto T, Osawa M, Kamata J, Itoh M, Nakai K, Habano W, Kiuchi T, Yamamori S, Hiramori K. 1998. Polymorphism of the apolipoprotein E and angiotensin I converting enzyme genes in Japanese patients with myocardial infarction. Coron Artery Dis. 9:329-34.
Nielsen P E, Egholm M, Berg R H, Buchardt O. 1991. Sequence-selective recognition of DNA by strand displacement with a thymine-substituted polyamide. Science. 254:1497-500.
Norlund L, Holm J, Zoller B, Ohlin A K. 1997. A common thrombomodulin amino acid dimorphism is associated with myocardial infarction. Thromb Haemost. 77:248-51.
Orita M, Iwahana H, Kanazawa H, Hayashi K, Sekiya T. 1989. Detection of polymorphisms of human DNA by gel electrophoresis as single-strand conformation polymorphisms. Proc Natl Acad Sci USA. 86:2766-70.
Pajukanta P, Cargill M, Viitanen L, Nuotio I, Kareinen A, Perola M, Terwilliger J D, Kempas E, Daly M, Lilja H, Rioux J D, Brettin T, Viikari J S, Ronnemaa T, Laakso M, Lander E S, Peltonen L. 2000. Two loci on chromosomes 2 and X for premature coronary heart disease identified in early- and late-settlement populations of Finland. Am J Hum Genet. 67:1481-93.
Park H Y, Nabika T, Jang Y, Kwon H M, Cho S Y, Masuda J. 2002. Association of G-33A polymorphism in the thrombomodulin gene with myocardial infarction in Koreans. Hypertens Res. 25:389-94.
Park J E, Lee W H, Hwang T H, Chu J A, Kim S, Choi Y H, Kim J S, Kim D K, Lee S H, Hong K P, Seo J D, Lee W R. 2000. Aging affects the association between endothelial nitric oxide synthase gene polymorphism and acute myocardial infarction in the Korean male population. Korean J Intern Med. 15:65-70.
Pastinen T, Perola M, Niini P, Terwilliger J, Salomaa V, Vartiainen E, Peltonen L, Syvanen A. 1998. Array-based multiplex analysis of candidate genes reveals two independent and additive genetic risk factors for myocardial infarction in the Finnish population. Hum Mol Genet. 7:1453-62.
Peltonen L, Jalanko A, Varilo T. Molecular genetics of the Finnish disease heritage. 1999. Hum Mol Genet. 8: 1913-23.
Petersen M, Wengel J. 2003. LNA: a versatile tool for therapeutics and genomics. Trends Biotechnol. 21:74-81.
Rauramaa R, Vaisanen S B, Luong L A, Schmidt-Trucksass A, Penttila I M, Bouchard C, Toyry J, Humphries S E. 2000. Stromelysin-1 and interleukin-6 gene promoter polymorphisms are determinants of asymptomatic carotid artery atherosclerosis. Arterioscler Thromb Vasc Biol. 20:2657-62.
Rewers M, Kamboh M I, Hoag S, Shetterly S M, Ferrell R E, Hamman R F. 1994. ApoA-IV polymorphism associated with myocardial infarction in obese NIDDM patients. The San Luis Valley Diabetes Study. Diabetes. 43:1485-9.
Ross R. 1999. Atherosclerosis—an inflammatory disease. Engl J. Med. 340:115-26.
Rundek T, Elkind M S, Pittman J, Boden-Albala B, Martin S, Humphries S E, Juo S H, Sacco R L. 2002. Carotid intima-media thickness is associated with allelic variants of stromelysin-1, interleukin-6, and hepatic lipase genes: the Northern Manhattan Prospective Cohort Study. Stroke. 33:1420-3.
Saiki R K, Bugawan T L, Horn G T, Mullis K B, Erlich H A. 1986. Analysis of enzymatically amplified beta-globin and HLA-DQ alpha DNA with allele-specific oligonucleotide probes. Nature. 324:163-6.
Sajantila A, Salem A H, Savolainen P, Bauer K, Gierig C, Paabo S. 1996. Paternal and maternal DNA lineages reveal a bottleneck in the founding of the Finnish population. Proc Natl Acad Sci USA. 93: 12035-9.
Salonen J T, Malin R, Tuomainen T P, Nyyssonen K, Lakka T A, Lehtimaki T. 1999. Polymorphism in high density lipoprotein paraoxonase gene and risk of acute myocardial infarction in men: prospective nested case-control study. BMJ. 319:487-9.
Salonen J T, Salonen R, Seppanen K, Rauramaa R, Tuomilehto J. 1991. High density lipoprotein, HDL2 and HDL3 subfractions and the risk of acute myocardial infarction: a prospective population study in Eastern Finnish men. Circulation 84:129-139.
Salonen J T, Ylä-Herttuala S, Yamamoto R, Butler S, Korpela H, Salonen R, Nyyssönen K, Palinski W, Witztum J L. 1992. Autoantibody against oxidised LDL and progression of carotid atherosclerosis. Lancet 339:883-887.
Salonen J T. 1988. Is there a continuing need for longitudinal epidemiologic research? The Kuopio Ischaemic Heart Disease Risk Factor Study. Ann Clin Res 20: 46-50.
Sanghera D K, Saha N, Aston C E, Kamboh M I. 1997. Genetic polymorphism of paraoxonase and the risk of coronary heart disease. Arterioscler Thromb Vasc Biol. 17:1067-73.
Sen-Banerjee S, Siles X, Campos H. 2000. Tobacco smoking modifies association between Gln-Arg192 polymorphism of human paraoxonase gene and risk of myocardial infarction. Arterioscler Thromb Vasc Biol. 20:2120-6.
Senti M, Tomas M, Vila J, Marrugat J, Elosua R, Sala J, Masia R. 2001. Relationship of age-related myocardial infarction risk and Gln/Arg 192 variants of the human paraoxonasel gene: the REGICOR study. Atherosclerosis. 156:443-9.
Serrato M, Marian A J. 1995. A variant of human paraoxonase/arylesterase (HUMPONA) gene is a risk factor for coronary artery disease. J Clin Invest. 96:3005-8.
Sevon P. 2004. Algorithms for Association-Based Gene Mapping. PhD Thesis, Series of Publication A, Report A-2004-4, University of Helsinki, Department of Computer Science, 101 pages, ISBN 952-10-1926-3 (pdf).
Sheffield V C, Cox D R, Lerman L S, Myers R M. 1989. Attachment of a 40-base-pair G+C-rich sequence (GC-clamp) to genomic DNA fragments by the polymerase chain reaction results in improved detection of single-base changes. Proc Natl Acad Sci U S A. 86:232-6.
Shimasaki Y, Yasue H, Yoshimura M, Nakayama M, Kugiyama K, Ogawa H, Harada E, Masuda T, Koyama W, Saito Y, Miyamoto Y, Ogawa Y, Nakao K. 1998. Association of the missense Glu298Asp variant of the endothelial nitric oxide synthase gene with myocardial infarction. J Am Coll Cardiol. 31:1506-10.
Sholtz R I, Rosenman R H, Brand R J. 1975. The relationship of reported parental history to the incidence of coronary heart disease in the Western Collaborative Group Study. Am J Epidemiol 102: 350-6.
Simon R. 2003. Using DNA microarrays for diagnostic and prognostic prediction. Expert Rev Mol Diagn. 3:587-95.
Smith D B, Flavell R B. 1977. Nucleotide sequence organisation in the rye genome. Biochim Biophys Acta. 474:82-97.
Smithies O, Gregg R G, Boggs S S, Koralewski M A, Kucherlapati R S. 1985. Insertion of DNA sequences into the human chromosomal beta-globin locus by homologous recombination. Nature. 317:230-4.
Stamler J, Greenland P, Neaton J D. 1998. The established major risk factors underlying epidemic coronary and cardiovascular disease. CVD Prevention 1: 82-97.
Stary H C, Blankenhorn D H, Chandler A B, Glagov S, Insull W Jr, Richardson M, Rosenfeld M E, Schaffer S A, Schwartz C J, Wagner W D, et al. 1992. A definition of the intima of human arteries and of its atherosclerosis-prone regions. A report from the Committee on Vascular Lesions of the Council on Arteriosclerosis, American Heart Association. Circulation 85: 391-405.
Stary H C, Chandler A B, Dinsmore R E, Fuster V, Glagov S, Insull W Jr, Rosenfeld M E, Schwartz C J, Wagner W D, Wissler R W. 1995. A definition of advanced types of atherosclerotic lesions and a histological classification of atherosclerosis. A report from the Committee on Vascular Lesions of the Council on Arteriosclerosis, American Heart Association. Circulation 92: 1355-74.
Stary H C, Chandler A B, Glagov S, Guyton J R, Insull W Jr, Rosenfeld M E, Schaffer S A, Schwartz C J, Wagner W D, Wissler R W. 1994. A definition of initial, fatty streak, and intermediate lesions of atherosclerosis. A report from the Committee on Vascular Lesions of the Council on Arteriosclerosis, American Heart Association. Circulation 89: 2462-78.
Stary H C. 2000. Lipid and macrophage accumulations in arteries of children and the development of atherosclerosis. Am J Clin Nutr 72(5 Suppl): S1297-1306.
Stein C A, Cohen J S. 1988. Oligodeoxynucleotides as inhibitors of gene expression: a review. Cancer Res. 48:2659-68.
Steinberg D. Lewis A. 1997. Conner Memorial Lecture. Oxidative modification of LDL and atherogenesis. Circulation 95: 1062-71.
Stephens J W, Humphries S E. 2003. The molecular genetics of cardiovascular disease: clinical implications. J Intern Med. 253:120-7.
Syvanen A-C. 2001. Accessing genetic variation: Genotyping single nucleotide polymorphisms. Nature Reviews Genetics. 2:930-942.
Terashima M, Akita H, Kanazawa K, Inoue N, Yamada S, Ito K, Matsuda Y, Takai E, Iwai C, Kurogane H, Yoshida Y, Yokoyama M. 1999. Stromelysin promoter 5A/6A polymorphism is associated with acute myocardial infarction. Circulation. 99:2717-9.
Thomas K R, Capecchi M R. 1987. Site-directed mutagenesis by gene targeting in mouse embryo-derived stem cells. Cell. 51:503-12.
Thompson S, Clarke A R, Pow A M, Hooper M L, Melton D W. 1989. Germ line transmission and expression of a corrected HPRT gene produced by gene targeting in embryonic stem cells. Cell. 56:313-21.
Toivonen H T, Onkamo P, Vasko K, Ollikainen V, Sevon P, Mannila H, Herr M, Kere J. 2000. Data mining applied to linkage disequilibrium mapping. Am J Hum Genet. 67:133-45.
Tremoli E, Camera M, Toschi V, Colli S. 1999. Tissue factor in atherosclerosis. Atherosclerosis. 144:273-83.
Tun A, Khan I A. Myocardial infarction with normal coronary arteries: the pathologic and clinical perspectives. Angiology. 2001; 52:299-304.
Tuomainen T P, Kontula K, Nyyssonen K, Lakka T A, Helio T, Salonen J T. 1999. Increased risk of acute myocardial infarction in carriers of the hemochromatosis gene Cys282Tyr mutation: a prospective cohort study in men in eastern Finland. Circulation. 100:1274-9.
Waller B F, Fry E T, Hermiller J B, Peters T, Slack J D. 1996. Nonatherosclerotic causes of coronary artery narrowing—Part II. Clin Cardiol. 19:587-91.
Waller B F, Fry E T, Hermiller J B, Peters T, Slack J D. 1996. Nonatherosclerotic causes of coronary artery narrowing—Part I. Clin Cardiol. 19:509-12.
Waller B F, Fry E T, Hermiller J B, Peters T, Slack J D. 1996. Nonatherosclerotic causes of coronary artery narrowing—Part III. Clin Cardiol. 19:656-61.
van der Krol A R, Mol J N, Stuitje A R. 1988. Modulation of eukaryotic gene expression by complementary RNA or DNA sequences. Biotechniques 6:958-76.
Wang Q, Rao S, Shen G Q, Li L, Molitemo D J, Newby L K, Rogers W J, Cannata R, Zirzow E, Elston R C, Topol E J. 2004. Premature myocardial infarction novel susceptibility locus on chromosome 1P34-36 identified by genomewide linkage analysis. Am J Hum Genet. 74:262-71.
Wei D, Shan J, Chen Z, Shi Y. 2002. The G894T mutation of the endothelial nitric oxide synthase gene is associated with coronary atherosclerotic heart disease in Chinese. Zhonghua Yi Xue Yi Chuan Xue Za Zhi. 19:471-4.
Virmani R, Burke A P, Farb A. 2001. Sudden cardiac death. Cardiovasc Pathol.10:211-8.
Wong W M, Hawe E, Li L K, Miller G J, Nicaud V, Pennacchio L A, Humphries S E, Talmud P J. 2003. Apolipoprotein AIV gene variant S347 is associated with increased risk of coronary heart disease and lower plasma apolipoprotein AIV levels. Circ Res. 92:969-75.
Wood D. 2001. Established and emerging cardiovascular risk factors. Am Heart J 141(2 Suppl): S49-57.
World Health Organization.2004. Cardiovascular diseases: prevention and control.

Information sheet. http://www.who.int/dietphysicalactivity/media/en/gsfs-cvd.pdf (Accessed 14.07.04).
Wu K K, Aleksic N, Ahn C, Boerwinkle E, Folsom A R, Juneja H; Atherosclerosis Risk in Communities Study (ARIC) Investigators. 2001. Thrombomodulin Ala455Val polymorphism and risk of coronary heart disease. Circulation. 103:1386-9.
Zhan M, Zhou Y, Han Z. 2003. Plasminogen activator inhibitor-1 4G/5G gene polymorphism in patients with myocardial or cerebrovascular infarction in Tianjin, China. Chin Med J. 116:1707-10.
Zon G. 1988. Oligonucleotide analogues as potential chemotherapeutic agents. Pharm Res. 5:539-49.
Zon G. 1988. Oligonucleotide analogues as potential chemotherapeutic agents. Pharm Res. 5:539-49.

1. A method for identification of an individual who has an altered risk of or susceptibility for developing CHD (i.e. coronary heart disease) or CHD death, the method comprising the steps of:
a) providing a biological sample taken from said individual;
b) collecting personal and clinical information of said individual;
c) determining the nucleotides present in one or several of the polymorphic sites as set forth in tables 3, 4, 5, 7 and 8 in said individual's nucleic acid; and
d) combining the data obtained from step c) with personal and clinical information obtained from step b) to assess the risk of an individual to develop CHD or CHD death.

2. The method according to claim 1, wherein the altered risk is an increased risk of CHD or CHD death.

3. The method according to claim 1, wherein the altered risk is a decreased risk of CHD or CHD death.

4. The method according to claim 1, wherein the polymorphic sites are those present in the haplotypes presented in tables 4, 5 and 8.

5. The method according to claim 1, wherein the polymorphic sites are associated with the SNP markers set forth in tables 3, 4, 5, 7 and 8.

6. The method according to claim 5, wherein the polymorphic sites are in complete linkage disequilibrium with the SNP markers set forth in tables 3, 4, 5, 7 and 8.

7. The method according to claim 6, wherein the polymorphic sites are in complete linkage disequilibrium in the population in which said method is used.

8. A method for identification of an individual who has an altered risk of or susceptibility for developing CHD or CHD death, the method comprising the steps of
a) providing a biological sample taken from said individual
b) determining the nucleotides present in one or several of the polymorphic sites as set forth in tables 3, 4, 5, 7 and 8 in said individual's nucleic acid
c) combining the data obtained from step b) to assess the risk of an individual to develop CHD death

9. The method according to claim 8, wherein the altered risk is an increased risk of CHD or CHD death.

10. The method according to claim 8, wherein the altered risk is a decreased risk of CHD or CHD death.

11. The method according to claim 8, wherein the polymorphic sites are those present in the haplotypes presented in tables 4, 5 and 8.

12. The method according to claim 8, wherein the polymorphic sites are associated with the SNP markers set forth in tables 3, 4, 5, 7 and 8.

13. The method according to claim 12, wherein the polymorphic sites are in complete linkage disequilibrium with the SNP markers set forth in tables 3, 4, 5, 7 and 8.

14. The method according to claim 13, wherein the polymorphic sites are in complete linkage disequilibrium in the population in which the said method is used.

15. The method according to claim 1, wherein said one or several polymorphic sites reside within a CHD risk gene or genes as set forth in table 6.

16. The method according to claim 1, wherein the CHD risk genes reside in the genome region which is defined by the haplotype pattern mining analysis, the genes set forth in tables 4, 5 and 8.

17. The method according to claim 1, wherein the polymorphic sites are associated with the haplotype regions, haplotypes or SNP markers defining the haplotypes set forth in tables 4, 5 and 8.

18. The method according to claim 17, wherein the polymorphic sites are in complete linkage disequilibrium with the haplotype regions, haplotypes or SNP markers defining the haplotypes set forth in tables 4, 5 and 8.

19. The method according to claim 18, wherein the polymorphic sites are in complete linkage disequilibrium in the population in which said method is used.

20. The method according to claim 5, wherein one or several of the SNP markers are selected from the group consisting of the following haplotypes or individual SNPs:
a) rs1095493 (A/G) (SEQ ID NO:153), rs1902021 (A/G) (SEQ ID NO:695), rs722087 (A/G) (SEQ ID NO:1253) and rs962580 (C/G) (SEQ ID NO:1473) defining the haplotype GAAC (or nucleotides from the complementary strand);
b) rs207098 (A/C) (SEQ ID NO:757), rs207097 (A/G) (SEQ ID NO:756) and rs10484411 (A/G) (SEQ ID NO:46) defining the haplotype CGG (or nucleotides from the complementary strand);
c) rs6952184 (C/T) (SEQ ID NO:1205) and rs7807993 (A/C) (SEQ ID NO:1327) defining the haplotype TC (or nucleotides from the complementary strand);
d) rs223921 (C/G) (SEQ ID NO:813) and rs10489033 (A/G) (SEQ ID NO:73) defining the haplotype CA (or nucleotides from the complementary strand);
e) rs10521300 (C/T) (SEQ ID NO:436), rs1112899 (C/T) (SEQ ID NO:454), rs8049155 (C/T) (SEQ ID NO:1356), rs1543921 (A/G) (SEQ ID NO:608) and rs9302658 (C/G) (SEQ ID NO:1420) defining the haplotype TTTGG (or nucleotides from the complementary strand);
f) rs10512615 (A/G) (SEQ ID NO:345) defining the risk allele A;
g) rs10519855 (A/G) (SEQ ID NO:418) defining the risk allele G

21. The method according to claim 1, wherein one or several of the SNP markers are selected from the group consisting of the following haplotypes or individual SNPs:
a) rs10495493 (A/G) (SEQ ID NO:153), rs1902021 (A/G) (SEQ ID NO:695), rs722087 (A/G) (SEQ ID NO:1253) and rs962580 (C/G) (SEQ ID NO:1473) defining the haplotype GAAC (or nucleotides from the complementary strand);
b) rs207098 (A/C) (SEQ ID NO:757), rs207097 (A/G) (SEQ ID NO:756) and rs10484411 (A/G) (SEQ ID NO:46) defining the haplotype CGG (or nucleotides from the complementary strand);
c) rs6952184 (C/T) (SEQ ID NO:1205) and rs7807993 (A/C) (SEQ ID NO:1327) defining the haplotype TC (or nucleotides from the complementary strand);
d) rs223921 (C/G) (SEQ ID NO:813) and rs10489033 (A/G) (SEQ ID NO:73) defining the haplotype CA (or nucleotides from the complementary strand);
e) rs10521300 (C/T) (SEQ ID NO:436), rs1112899 (C/T) (SEQ ID NO:454), rs8049155 (C/T) (SEQ ID NO:1356), rs1543921 (A/G) (SEQ ID NO:608) and rs9302658 (C/G) (SEQ ID NO:1420) defining the haplotype TTTGG (or nucleotides from the complementary strand);
f) rs10519855 (A/G) (SEQ ID NO:418) defining the risk allele G;
g) rs10515605 (A/G) (SEQ ID NO:370) defining the risk allele A

22. The method according to claim 1, wherein one or several of the SNP markers are selected from the group consisting of the following haplotypes or individual SNPs:
a) rs10495493 (A/G) (SEQ ID NO:153), rs1902021 (A/G) (SEQ ID NO:695), rs722087 (A/G) (SEQ ID NO:1253) and rs962580 (C/G) (SEQ ID NO:1473) defining the haplotype GAAC (or nucleotides from the complementary strand);
b) rs207098 (A/C) (SEQ ID NO:757), rs207097 (A/G) (SEQ ID NO:756) and rs10484411 (A/G) (SEQ ID NO:46) defining the haplotype CGG (or nucleotides from the complementary strand);
c) rs6952184 (C/T) (SEQ ID NO:1205) and rs7807993 (A/C) (SEQ ID NO:1327) defining the haplotype TC (or nucleotides from the complementary strand);
d) rs223921 (C/G) (SEQ ID NO:813) and rs10489033 (A/G) (SEQ ID NO:73) defining the haplotype CA (or nucleotides from the complementary strand);
e) rs10521300 (C/T) (SEQ ID NO:436), rs1112899 (C/T) (SEQ ID NO:454), rs8049155 (C/T) (SEQ ID NO:1356), rs1543921 (A/G) (SEQ ID NO:608) and rs9302658 (C/G) (SEQ ID NO:1420) defining the haplotype TTTGG (or nucleotides from the complementary strand);
f) rs10519855 (A/G) (SEQ ID NO:418) defining the risk allele G;
g) rs10515605 (A/G) (SEQ ID NO:370) defining the risk allele A

23. The method according to claim 1, wherein the non-genetic information obtained from step b) contains age and the mean plasma insulin of the individual.

24. The method according to claim 1, wherein one or several of the SNP markers are selected from the group consisting of the following haplotypes:
a) rs2796249 (C/T) (SEQ ID NO:924), rs6667619 (G/T) (SEQ ID NO:1173), rs1932818 (C/T) (SEQ ID NO:711) and rs6663269 (C/G) (SEQ ID NO:1172) defining the haplotype CTCC;
b) rs6663269 (C/G) (SEQ ID NO:1172), rs1160530 (C/T) (SEQ ID NO:456) and rs631802 (A/G) (SEQ ID NO:1151) defining the haplotype CCG;
c) rs6424260 (A/G) (SEQ ID NO:1152), rs6673130 (A/G) (SEQ ID NO:1174), rs7534667 (C/G) (SEQ ID NO:1292) and rs3766476 (C/T) (SEQ ID NO:1011) defining the haplotype AGGC;
d) rs10489416 (C/T) (SEQ ID NO:78), rs2202094 (G/T) (SEQ ID NO:800), rs218390 (A/T) (SEQ ID NO:790), rs218385 (C/T) (SEQ ID NO:789) and rs218381 (A/G) (SEQ ID NO:788) defining the haplotype TGTTA;
e) rs10520241 (C/T) (SEQ ID NO:420), rs6723256 (C/G) (SEQ ID NO:1179), rs1430635 (A/G) (SEQ ID NO:553) and rs1864549 (G/T) (SEQ ID NO:672) defining the haplotype TCGG;
f) rs1228055 (A/G) (SEQ ID NO:460), rs1228054 (C/T) (SEQ ID NO:459), rs1922035 (C/T) (SEQ ID NO:705) and rs6746500 (C/T) (SEQ ID NO:1182) defining the haplotype ATCT;
g) rs2345512 (C/G) (SEQ ID NO:839), rs7570727 (A/G) (SEQ ID NO:1297), rs2345516 (C/T) (SEQ ID NO:840) and rs2345518 (A/C) (SEQ ID NO:842) defining the haplotype CGCA;
h) rs10495493 (A/G) (SEQ ID NO:153), rs1902021 (A/G) (SEQ ID NO:695), rs722087 (A/G) (SEQ ID NO:1253) and rs962580 (C/G) (SEQ ID NO:1473) defining the haplotype GAAC;
i) rs10511164 (A/G) (SEQ ID NO:328), rs832064 (A/T) (SEQ ID NO:1370), rs937128 (C/T) (SEQ ID NO:1454) and rs1546223 (C/T) (SEQ ID NO:610) defining the haplotype AATT;
j) rs4686145 (A/C) (SEQ ID NO:1083), rs6768216 (C/T) (SEQ ID NO:1186), rs162803 (C/G) (SEQ ID NO:644) and rs10514663 (C/T) (SEQ ID NO:359) defining the haplotype CTGC;
k) rs2201151 (G/T) (SEQ ID NO:799), rs4857302 (A/C) (SEQ ID NO:1100), rs1492054 (C/T) (SEQ ID NO:586) and rs10511164 (A/G) (SEQ ID NO:328) defining the haplotype GCCA;
l) rs223921 (C/G) (SEQ ID NO:813) and rs10489033 (A/G) (SEQ ID NO:73) defining the haplotype CA;
m) rs2703134 (C/G) (SEQ ID NO:911), rs2703133 (C/G) (SEQ ID NO:910), rs2703132 (C/G) (SEQ ID NO:909), rs2703137 (G/T) (SEQ ID NO:912) and rs2645690 (C/G) (SEQ ID NO:907) defining the haplotype GCCTG;
n) rs9307776 (A/T) (SEQ ID NO:1427), rs10516735 (C/T) (SEQ ID NO:390), rs2055178 (A/G) (SEQ ID NO:754) and rs1353387 (C/T) (SEQ ID NO:500) defining the haplotype ACGT;
o) rs10520435 (C/T) (SEQ ID NO:423), rs1379987 (A/G) (SEQ ID NO:519) and rs2100684 (C/T) (SEQ ID NO:766) defining the haplotype CAC;
p) rs409336 (A/C) (SEQ ID NO:1044), rs2472649 (C/T) (SEQ ID NO:887), rs450373 (A/G) (SEQ ID NO:1065) and rs484608 (A/G) (SEQ ID NO:1099) defining the haplotype ACAA;
q) rs10516922 (C/T) (SEQ ID NO:392), rs10516923 (A/G) (SEQ ID NO:393), rs10516924 (A/C) (SEQ ID NO:394), rs10516925 (C/T) (SEQ ID NO:395) and rs10516926 (C/T) (SEQ ID NO:396) defining the haplotype CAATC;
r) rs10515605 (A/G) (SEQ ID NO:370), rs7709159 (C/T) (SEQ ID NO:1317) and rs10515609 (C/T) (SEQ ID NO:371) defining the haplotype ACT;
s) rs2301081 (A/C) (SEQ ID NO:832), rs1966580 (C/T) (SEQ ID NO:718), rs2301086 (C/T) (SEQ ID NO:833), rs1346572 (C/T) (SEQ ID NO:497) and rs10514263 (A/C) (SEQ ID NO:355) defining the haplotype ATCTA;
t) rs10515538 (C/T) (SEQ ID NO:366), rs10515541 (C/T) (SEQ ID NO:367), rs10515542 (C/T) (SEQ ID NO:368) and rs358635 (A/G) (SEQ ID NO:995) defining the haplotype CTCA;
u) rs207098 (A/C) (SEQ ID NO:757), rs207097 (A/G) (SEQ ID NO:756) and rs10484411 (A/G) (SEQ ID NO:46) defining the haplotype CGG;
v) rs7766687 (C/T) (SEQ ID NO:1323), rs6922836 (G/T) (SEQ ID NO:1202), rs10498950 (C/T) (SEQ ID NO:184) and rs6934503 (C/G) (SEQ ID NO:1203) defining the haplotype TTTG;
w) rs9297050 (A/G) (SEQ ID NO:1414), rs2206144 (C/T) (SEQ ID NO:803), rs4716220 (A/G) (SEQ ID NO:1084) and rs214614 (A/G) (SEQ ID NO:775) defining the haplotype GCAA;
x) rs6952184 (C/T) (SEQ ID NO:1205) and rs7807993 (A/C) (SEQ ID NO:1327) defining the haplotype TC;
y) rs10487391 (A/G) (SEQ ID NO:65), rs3757798 (A/G) (SEQ ID NO:1007) and rs3757797 (A/C) (SEQ ID NO:1006) defining the haplotype GAA;
z) rs10499328 (G/T) (SEQ ID NO:187), rs4418248 (C/T) (SEQ ID NO:1062) and rs6952184 (C/T) (SEQ ID NO:1205) defining the haplotype GCT;
aa) rs10280843 (A/G) (SEQ ID NO:17), rs10241344 (C/G) (SEQ ID NO:10) and rs13073 (A/G) (SEQ ID NO:469) defining the haplotype GGG;
bb) rs1573311 (C/T) (SEQ ID NO:625), rs1037701 (A/C) (SEQ ID NO:25), rs1265145 (C/T) (SEQ ID NO:463), rs1265151 (C/T) (SEQ ID NO:464) and rs10505019 (A/T) (SEQ ID NO:247) defining the haplotype CCCTT;
cc) rs4467935 (A/G) (SEQ ID NO:1064), rs10503569 (C/T) (SEQ ID NO:226), rs7819568 (A/G) (SEQ ID NO:1328), rs10503570 (A/G) (SEQ ID NO:227) and rs4240184 (C/T) (SEQ ID NO:1054) defining the haplotype ACAAC;
dd) rs10505017 (C/T) (SEQ ID NO:246), rs1111908 (A/C) (SEQ ID NO:452), rs7012174 (C/T) (SEQ ID NO:1216) and rs1573311 (C/T) (SEQ ID NO:625) defining the haplotype TATC;
ff) rs4403471 (A/G) (SEQ ID NO:1061), rs4743487 (G/T) (SEQ ID NO:1087), rs10512291 (A/G) (SEQ ID NO:338), rs1337690 (C/G) (SEQ ID NO:489) and rs10512292 (C/T) (SEQ ID NO:339) defining the haplotype AGGGT;
gg) rs10491759 (A/T) (SEQ ID NO:114), rs8192981 (C/T) (SEQ ID NO:1364), rs549130 (C/T) (SEQ ID NO:1123), rs1590405 (C/T) (SEQ ID NO:632) and rs489504 (C/T) (SEQ ID NO:1103) defining the haplotype ACCCT;
hh) rs1541018 (C/T) (SEQ ID NO:606), rs7897982 (C/T) (SEQ ID NO:1342), rs10508463 (A/G) (SEQ ID NO:278), rs10508464 (A/G) (SEQ ID NO:279) and rs10508465 (C/T) (SEQ ID NO:280) defining the haplotype CCAAC;
ii) rs7070112 (A/T) (SEQ ID NO:1225), rs1336507 (G/T) (SEQ ID NO:487), rs1336508 (C/G) (SEQ ID NO:488), rs9325491 (A/G) (SEQ ID NO:1451) and rs877816 (A/G) (SEQ ID NO:1380) defining the haplotype ATCGA;
jj) rs10501362 (C/T) (SEQ ID NO:197), rs540505 (A/T) (SEQ ID NO:1120), rs2957177 (C/T) (SEQ ID NO:967) and rs493461 (A/C) (SEQ ID NO:1106) defining the haplotype TATA;
kk) rs10501869 (A/G) (SEQ ID NO:199), rs964183 (A/G) (SEQ ID NO:1478), rs10501870 (A/T) (SEQ ID NO:200) and rs964646 (A/G) (SEQ ID NO:1479) defining the haplotype AGTG;
ll) rs605954 (A/G) (SEQ ID NO:1144), rs527529 (C/T) (SEQ ID NO:1113), rs590105 (G/T) (SEQ ID NO:1131), rs671544 (A/T) (SEQ ID NO:1178) and rs536412 (C/G) (SEQ ID NO:1119) defining the haplotype GTGTC;
mm) rs10505953 (G/T) (SEQ ID NO:257), rs976436 (C/T) (SEQ ID NO:1491), rs10505954 (A/G) (SEQ ID NO:258) and rs7296881 (G/T) (SEQ ID NO:1269) defining the haplotype GTGT;
nn) rs2961370 (A/G) (SEQ ID NO:968), rs7305762 (C/T) (SEQ ID NO:1272), rs10505838 (G/T) (SEQ ID NO:253) and rs7300261 (A/C) (SEQ ID NO:1271) defining the haplotype ATTA;
oo) rs772556 (C/T) (SEQ ID NO:1321), rs699585 (G/T) (SEQ ID NO:1212), rs699586 (C/T) (SEQ ID NO:1213) and rs10506468 (A/G) (SEQ ID NO:263) defining the haplotype TTCG;
pp) rs9316159 (C/T) (SEQ ID NO:1441), rs9316160 (A/G) (SEQ ID NO:1442), rs10507537 (A/G) (SEQ ID NO:271) and rs7989399 (C/T) (SEQ ID NO:1349) defining the haplotype TAAT;
qq) rs1340313 (C/T) (SEQ ID NO:491), rs1340321 (G/T) (SEQ ID NO:492), rs10507707 (C/T) (SEQ ID NO:272), rs10507708 (A/G) (SEQ ID NO:273) and rs10507710 (A/G) (SEQ ID NO:274) defining the haplotype CGCAG;
rr) rs744509 (A/G) (SEQ ID NO:1285), rs744511 (A/G) (SEQ ID NO:1286), rs10483534 (A/G) (SEQ ID NO:30) and rs7148846 (A/C) (SEQ ID NO:1235) defining the haplotype GGAC;
ss) rs10483732 (A/G) (SEQ ID NO:39), rs718028 (A/G) (SEQ ID NO:1239) and rs10483734 (C/T) (SEQ ID NO:40) defining the haplotype AGT;
tt) rs2181663 (A/G) (SEQ ID NO:787), rs2401841 (C/G) (SEQ ID NO:869), rs10484015 (A/G) (SEQ ID NO:42), rs10484016 (C/T) (SEQ ID NO:43) and rs7350724 (A/G) (SEQ ID NO:1282) defining the haplotype ACGCA;
uu) rs2255994 (C/T) (SEQ ID NO:819), rs10506993 (C/G) (SEQ ID NO:264), rs1478199 (C/T) (SEQ ID NO:578) and rs1478200 (A/G) (SEQ ID NO:579) defining the haplotype CCCA;
vv) rs10519249 (C/T) (SEQ ID NO:412), rs10519250 (A/G) (SEQ ID NO:413), rs10519251 (A/G) (SEQ ID NO:414), rs2413992 (A/G) (SEQ ID NO:871) and rs2413996 (A/C) (SEQ ID NO:872) defining the haplotype CAGAC;
ww) rs10521300 (C/T) (SEQ ID NO:436), rs1112899 (C/T) (SEQ ID NO:454), rs8049155 (C/T) (SEQ ID NO:1356), rs1543921 (A/G) (SEQ ID NO:608) and rs9302658 (C/G) (SEQ ID NO:1420) defining the haplotype TTTGG;
xx) rs4783294 (C/T) (SEQ ID NO:1090), rs4783295 (A/G) (SEQ ID NO:1091), rs10492864 (A/T) (SEQ ID NO:124) and rs9319579 (A/C) (SEQ ID NO:1447) defining the haplotype CGTC;
yy) rs1486747 (A/G) (SEQ ID NO:583), rs7226036 (A/C) (SEQ ID NO:1254) and rs7212568 (C/T) (SEQ ID NO:1252) defining the haplotype AAT;
zz) rs10502297 (A/G) (SEQ ID NO:205) and rs1940693 (C/T) (SEQ ID NO:714) defining the haplotype GC;
aaa) rs530205 (C/T) (SEQ ID NO:1116), rs646128 (A/C) (SEQ ID NO:1155), rs10502879 (A/G) (SEQ ID NO:216) and rs644731 (C/T) (SEQ ID NO:1154) defining the haplotype TAGC;
bbb) rs1431844 (C/T) (SEQ ID NO:556), rs10502791 (A/T) (SEQ ID NO:212), rs1431838 (A/G) (SEQ ID NO:554) and rs10502792 (C/G) (SEQ ID NO:213) defining the haplotype CTAC;
ccc) rs7247641 (C/G) (SEQ ID NO:1261), rs1056176 (G/T) (SEQ ID NO:441), rs2124902 (A/G) (SEQ ID NO:769), rs2262138 (C/T) (SEQ ID NO:822) and rs1004246 (A/G) (SEQ ID NO:2) defining the haplotype GGGCG;
ddd) rs845607 (C/T) (SEQ ID NO:1376), rs1099620 (C/T) (SEQ ID NO:449) and rs845591 (A/T) (SEQ ID NO:1375) defining the haplotype CTA;
eee) rs6088033 (C/T) (SEQ ID NO:1147) and rs1321425 (C/G) (SEQ ID NO:474) defining the haplotype TC;
fff) rs6028405 (C/T) (SEQ ID NO:1141), rs2206437 (A/T) (SEQ ID NO:804), rs1569608 (C/T) (SEQ ID NO:622), rs2179443 (C/G) (SEQ ID NO:786) and rs909874 (C/T) (SEQ ID NO:1387) defining the haplotype CACGT;
ggg) rs2824289 (A/T) (SEQ ID NO:930) and rs208921 (A/G) (SEQ ID NO:761) defining the haplotype AA;
hhh) rs132183 (C/T) (SEQ ID NO:476), rs720441 (C/G) (SEQ ID NO:1246), rs1983705 (C/T) (SEQ ID NO:722) and rs738743 (G/T) (SEQ ID NO:1283) defining the haplotype CCCT;
iii) rs2870458 (G/T) (SEQ ID NO:952) and rs2206024 (C/T) (SEQ ID NO:802) defining the haplotype TT

25. A method for assessing susceptibility or predisposition to CHD or CHD death in an individual, the method comprising determining alteration of expression levels of one or several of the genes of table 6 in the individual, wherein a difference in expression is indicative of susceptibility to CHD or CHD death.

26. The method according to claim 25, wherein alteration of expression levels is determined by assessing transcription levels of one or several of the genes of table 6 in the individual.

27. The method according to claim 25, wherein alteration of expression levels is determined by assessing translation of mRNAs encoded by one or several of the genes of table 6 in the individual.

28. A method for assessing susceptibility or predisposition to CHD or CHD death in an individual, the method comprising determining alteration of biological activity of one or several ot the polypeptides encoded by one or several of the genes of table 6 in the individual, wherein a difference in biological activity of one or several of the polypeptides is indicative of susceptibility to CHD or CHD death.

29. The method according to claim 28, wherein alteration of biological activity is determined by assessing structure of one or several ot the polypeptides encoded by one or several of the genes of table 6 in the individual.

30. The method according to claim 28, wherein alteration of biological activity is determined by assessing amount of one or several of the metabolites of a polypeptide or polypeptides encoded by one or several of the genes of table 6 in the individual.

31. The method according to claim 1, wherein the personal and clinical information, i.e. non-genetic information, concerns age, gender, behaviour patterns and habits, biochemical measurements, clinical measurements, obesity, the family history of CHD, cerebrovascular disease, other cardiovascular disease, hypercholesterolemia, obesity and diabetes, waist-to-hip circumference ratio (cm/cm), socioeconomic status, psychological traits and states, and the medical history of the subject.

32. The method according to claim 31, wherein the behaviour patterns and habits include tobacco smoking, physical activity, dietary intakes of nutrients, alcohol intake and consumption patterns and coffee consumption and quality.

33. The method according to claim 31, wherein the biochemical measurements include determining blood, serum or plasma VLDL, LDL, HDL or total cholesterol or triglycerides, apolipoprotein (a), fibrinogen, ferritin, transferrin receptor, C-reactive protein, glucose or insulin concentration.

34. The method according to claim 31, wherein the non-genetic measurements are those presented in table 9.

35. The method according to claim 31, wherein the non-genetic information contains age and the plasma insulin concentration of the subject.

36. The method according to claim 31 further comprising a step of calculating the risk of CHD or CHD death using a logistic regression equation as follows: Risk of CHD or CHD death=[1+e^{-(a+Σ(bi*Xi)]}⁻¹, where e is Napier's constant, Xᵢ are variables associated with the risk of CHD or CHD death, bi are coefficients of these variables in the logistic function, and a is the constant term in the logistic function

37. The method according to claim 36, wherein a and bᵢ are determined in the population in which the method is to be used.

38. The method according to claim 36, wherein Xi are selected among the variables that have been measured in the population in which the method is to be used.

39. The method according to claim 36, wherein Xi are selected among the SNP markers of tables 3, 4, 5, 7 and 8, among haplotype regions and haplotypes of tables 4, 5, 7 and 8 and among non-genetic variables of the invention.

40. The method according to claim 36, wherein bᵢ are between the values of −20 and 20 and/or wherein Xᵢ can have values between −99999 and 99999 or are coded as 0 (zero) or 1 (one).

41. The method according to claim 36, wherein i are between the values 0 (none) and 100,000.

42. The method according to claim 1, wherein subject's short term, median term, and/or long term risk of CHD or CHD death is predicted.

43. A method for treating a human or animal subject suffering from CHD or for treating complications of CHD, said method comprising a step of modulating or administering any of the polypeptides produced by the CHD risk genes as set forth in table 6.

44. A method for identifying compounds useful in prevention or treatment of CHD comprising determining the effect of a compound on biological networks and/or metabolic pathways related to one or several polypeptides encoded by CHD risk genes of table 6 in living cells; wherein a compound altering activity of one or several said biological networks and/or metabolic pathways is considered useful in prevention or treatment of CHD.

45. The method according to claim 44 comprising determining the effect of a compound on a biological activity of one or several polypeptides encoded by CHD risk genes of table 6 in living cells; wherein a compound altering biological activity of a polypeptide is considered useful in prevention and/or treatment of CHD.

46. A method for prevention or treatment of CHD comprising administering to a mammalian subject in need of such treatment an effective amount of a compound in a pharmaceutically acceptable carrier enhancing or reducing biological activity of one or several polypeptides encoded by CHD risk genes of table 6; and/or enhancing or reducing activity of one or several biological networks and/or metabolic pathways related to said polypeptides.

47. The method according to claim 44 comprising administering to a mammalian subject in need of such treatment an effective amount of a compound in a pharmaceutically acceptable carrier enhancing or reducing expression of one or several CHD risk genes of table 6; and/or enhancing or reducing the expression of one or several genes in biological networks and/or metabolic pathways related to polypeptides encoded by said CHD risk genes.

48. The method according to claim 46 comprising administering to a mammalian subject in need of such treatment an effective amount of a compound in a pharmaceutically acceptable carrier enhancing or reducing activity of one or several pathophysiological pathways involved in cardiovascular diseases and related to polypeptides encoded by CHD risk genes of table 6.

49. The method according to claim 46, said method comprising the steps of:
a) providing a biological sample taken from a subject;
b) determining the nucleotides present in one or several of the polymorphic sites associated with altered expression and/or biological activity and present in CHD risk genes of table 6 in said individual's nucleic acid; and
c) combining polymorphic site genotype data to select effective therapy for treating CHD in said subject.

50. The method according to claim 46, said method comprising the steps of:
a) providing a biological sample taken from a subject;
b) determining expression of one or several CHD risk genes of table 6 and/or determining biological activity of one or several polypeptides encoded by the CHD risk genes of table 6 in said individual's sample; and
c) combining the expression and/or biological activity data to select effective therapy for treating CHD in said subject.

51. The method according to claim 46, wherein said treatment is gene therapy or gene transfer.

52. The method according to claim 50, wherein said treatment comprises the transfer of one or several CHD risk genes of table 6 or variants, fragments or derivatives thereof.

53. The method according to claim 50, wherein said CHD risk genes of table 6 or variants, fragments or derivatives thereof are associated with reduced risk of CHD.

54. The method according to claim 50, wherein said treatment comprises treating regulatory regions and/or gene containing region of one or more CHD risk genes of table 6 or variants, fragments or derivatives thereof in somatic cells of said subject.

55. The method according to claim 50, wherein said treatment comprises treating regulatory regions and/or gene containing region of one or more CHD risk genes of table 6 or variants, fragments or derivatives thereof in stem cells.

56. The method according to claim 55, wherein said treatment comprises treating regulatory regions and/or gene containing region of one or more CHD risk genes of table 6 or variants, fragments or derivatives thereof in stem cells in tissues affected by cardiovascular diseases.

57. The method according to claim 46, wherein said compound is a recombinant polypeptide encoded by an CHD risk gene of table 6 or variant, fragment or derivative thereof.

58. The method according to claim 46, wherein said treatment is based on siRNA hybridising to mRNA and/or to hnRNA of a CHD risk gene of table 6.

59. The method according to claim 46, wherein said treatment is based on siRNA hybridising to mRNA and/or to hnRNA of one or several genes in biological networks and/or metabolic pathways related to polypeptides encoded by said CHD risk genes of table 6.

60. The method according to claim 46, wherein said method of treating is a dietary treatment or a vaccination.

61. The method according to claim 46 comprising a therapy restoring, at least partially, the observed alterations in biological activity of one or several polypeptides encoded by CHD risk genes of table 6 in said subject, when compared with CHD free healthy subjects.

62. The method according to claim 46 comprising a therapy restoring, at least partially, the observed alterations in expression of one or several CHD risk genes of table 6 in said subject, when compared with CHD free healthy subjects.

63. A method for monitoring the effectiveness of treatment of CHD in a human subject the method comprising measuring mRNA levels of CHD risk genes of table 6, and/or levels of polypeptides encoded by said CHD risk genes, and/or biological activity of polypeptides encoded by said CHD risk genes in a biological sample taken from said subject;
alteration of mRNA levels or polypeptide levels or biological activity of a polypeptide following treatment being indicative of the efficacy of the treatment.

64. A method for predicting the effectiveness of a given therapeutic for CHD such as AMI prevention or treatment in a given individual comprising screening for the presence or absence of the CHD death associated SNP markers, haplotypes or haplotype regions in one or several of the CHD risk genes of claim 15.

65. A method for predicting the effectiveness of a given therapeutic for CHD such as AMI prevention or treatment in a given individual, the method comprising the steps of:
a) providing a biological sample taken from a subject
b) determining the nucleotides present in one or several of the polymorphic sites as set forth in tables 3, 4, 5, 7 and 8 in said individual's nucleic acid; and
c) combining the SNP marker data to predict the effectiveness of a given therapeutic in an individual for CHD such as AMI prevention or treatment.

66. A method for diagnosing of a subtype of AMI in an individual having AMI, the method comprising the steps of:
a) providing a biological sample taken from a subject;
b) determining the nucleotides present in one or several of the SNP markers as set forth in tables 3, 4, 5, 7 and 8 in said individual's nucleic acid; and
d) combining the SNP marker data to assess the subtype of AMI of an individual.

67. The method according to claim 66, wherein said one or several SNP markers reside within a CHD risk gene or genes as set forth in table 6.

68. The method according to claim 66, wherein the CHD risk genes reside in the genome region which is defined by the haplotype pattern mining analysis, the genes and regions set forth in tables 4, 5 and 8.

69. The method according to claim 66, wherein the polymorphic sites are associated with the haplotype regions, haplotypes or SNP markers defining the haplotypes set forth in tables 4, 5 and 8.

70. The method according to claim 66, wherein the polymorphic sites are in complete linkage disequilibrium with the haplotype regions, haplotypes or SNP markers defining the haplotypes set forth in tables 4, 5 and 8.

71. The method according to claim 66, wherein the polymorphic sites are in complete linkage disequilibrium in the population in which the said method is used.

72. The method according to 46 further comprising a step of combining non-genetic information with the results obtained by a method comprising determining the effect of a compound on biological networks and/or metabolic pathways related to one or several polypeptides encoded by CHD risk genes of table 6 in living cells; wherein a compound altering activity of one or several said biological networks and/or metabolic pathways is considered useful in prevention or treatment of CHD.

73. The method according to claim 72, wherein the non-genetic information concerns age, gender, behaviour patterns and habits, biochemical measurements, clinical measurements, obesity, the family history of CHD, cerebrovascular disease, other cardiovascular disease, hypercholesterolemia, obesity and diabetes, waist-to-hip circumference ratio (cm/cm), socioeconomic status, psychological traits and states, and the medical history of the subject.

74. The method according to claim 72, wherein the behaviour patterns and habits include tobacco smoking, physical activity, dietary intakes of nutrients, alcohol intake and consumption patterns and coffee consumption and quality.

75. The method according to claim 72, wherein the biochemical measurements include determining blood, serum or plasma VLDL, LDL, HDL or total cholesterol or triglycerides, apolipoprotein (a), fibrinogen, ferritin, transferrin receptor, C-reactive protein, glucose, serum or plasma insulin concentration.

76. The method according to claim 72, wherein the non-genetic measurements are those presented in tables 7 and 8.

77. The method according to claim 72, wherein the non-genetic information contains the mean serum triglycerides of the subject.

78. A method for measuring CHD risk gene product protein expression, production or concentration in a biological sample taken from a subject, wherein said CHD risk gene is as defined in table 6, the method comprising the steps of:
a) providing a biological sample taken from a subject to be tested; and
b) detecting the expression, production or concentration of said protein in said sample, wherein altered expression, production or concentration indicates an altered risk of cardiovascular disease in said subject.

79. A test kit based on a method according to claim 1 for assessment of an altered risk of or susceptibility for CHD or CHD death in a subject.

80. A test kit for determining the nucleotides present in one or several of the SNP markers as set forth in tables 3, 4, 5, 7 and 8 in said individual's nucleic acid for assessment of an altered risk of CHD or CHD death in a subject.

81. A test kit for determining the nucleotides present in one or several of the SNP markers as set forth in tables 3, 4, 5, 7 and 8 in said individual's nucleic acid for assessment of an altered risk of CHD or CHD death in a subject, containing:
a) reagents and materials for assessing nucleotides present in one or several SNP markers as set forth in tables 3, 4, 5, 7 and 8; and
b) software to interpret the results of the determination.

82. The test kit according to claim 79 further comprising PCR primer set for amplifying nucleic acid fragments containing one or several SNP markers as set forth in tables 3, 4, 5, 7 and 8 from the nucleic acids of the subject.

83. The test kit according to claim 79 comprising a capturing nucleic acid probe set specifically binding to one or several SNP markers present in CHD death associated markers and haplotype regions as set forth in tables 3, 4, 5, 7 and 8.

84. The test kit according to claim 79 comprising a microarray or multiwell plate to assess the genotypes.

85. The test kit according to claim 79 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning CHD.

86. A test kit for detecting the presence of SNP markers in one or several of CHD risk genes as set forth in table 6 in a biological sample, wherein said SNP markers are more frequently present in a biological sample of a subject susceptible to CHD death compared to a sample from a subject not susceptible to CHD death, the kit comprising:
a) reagents and materials for assessing nucleotides present in SNP markers in one or several of CHD risk genes as set forth in table 6; and
b) software to interpret the results of the determination.

87. The test kit of claim 86 further comprising PCR primer set for amplifying nucleic acid fragments containing said SNP markers from CHD risk genes as set forth in table 6 from the nucleid acids of the subject.

88. The test kit of claim 86 comprising a capturing nucleic acid probe set specifically binding to one or several SNP markers present in CHD risk genes as set forth in table 6.

89. The test kit of claim 86 comprising a microarray or multiwell plate to assess the genotypes.

90. The test kit of claim 86 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning CHD.

91. A test kit based on a method according to claim 49.

92. The test kit of claim 91 further comprising PCR primer set for amplifying nucleic acid fragments containing said SNP markers from CHD risk genes as set forth in tables 3, 4, 5, 7 and 8 from the nucleid acids of the subject.

93. The test kit of claim 91 comprising a capturing nucleic acid probe set specifically binding to one or several SNP markers present in CHD risk genes as set forth in tables 3, 4, 5, 7 and 8.

94. The test kit of claim 91 comprising a microarray or multiwell plate to assess the genotypes.

95. The test kit of claim 91 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning CHD.

96. The test kit of claim 79, further comprising a marker set to assess the ancestry of an individual.

97. The test kit of claim 96 comprising a SNP marker set to assess the ancestry of an individual.

98. The test kit of claim 96 comprising a microsatellite marker set to assess the ancestry of an individual.

99. A method of claim 1 further comprising a marker set to assess the ancestry of an individual.

100. A method of claim 1 comprising a SNP marker set to assess the ancestry of an individual.

101. A method of claim 1 comprising a microsatellite marker set to assess the ancestry of an individual.


SEQUENCE LISTING (1503 sequences)

SEQ ID NO: 1 - DNA, 51 bp, Homo sapiens
caacctcgaatgtgtctcccaccaawagggtctcacgtaggattcaaacac

SEQ ID NO: 2 - DNA, 51 bp, Homo sapiens
tcccctgaggtagaccagagaagatrttgaagccatggaggggcaacaaac

SEQ ID NO: 3 - DNA, 51 bp, Homo sapiens
tccagctcattagagcagttagagaygcactgagcacgtaataacagatca

SEQ ID NO: 4 - DNA, 51 bp, Homo sapiens
aacattaatacagtgtttagttttaraaagtgtctgtttatacaccatgtc

SEQ ID NO: 5 - DNA, 51 bp, Homo sapiens
aggaagagagtatatttctcctcacygatttgggcttgaaatggccagcag

SEQ ID NO: 6 - DNA, 51 bp, Homo sapiens
aaaggccttctccaaccctagttaamgccatatctccaccttgtcccacca

SEQ ID NO: 7 - DNA, 51 bp, Homo sapiens
tagttagtcactagacaacccagtgyatcttaaactcccacttctggagaa

SEQ ID NO: 8 - DNA, 51 bp, Homo sapiens
atcacaagtatcactcattaaacatraaattacatttgttgataactggca

SEQ ID NO: 9 - DNA, 51 bp, Homo sapiens
aaacatacatgctttgactttacatyatctcatgaggcgttaacccagctg

SEQ ID NO: 10 - DNA, 51 bp, Homo sapiens
attctgttatagctgttaaaattaascatcaactataaaatcaggtaataa

SEQ ID NO: 11 - DNA, 51 bp, Homo sapiens
agttcttatacatcatttggagttargtttactgtctcctagtttctttga

SEQ ID NO: 12 - DNA, 51 bp, Homo sapiens
atgattaagttaaggaattatcgaaraccaatttgctcctcagtatgcaga

SEQ ID NO: 13 - DNA, 51 bp, Homo sapiens
ttaacataatgagaatctttatggamgaatttgcgacagctcaagaacatt

SEQ ID NO: 14 - DNA, 51 bp, Homo sapiens
taaaaaataaataagtaagataagcrcaggtcttcatccttctgttctgta

SEQ ID NO: 15 - DNA, 51 bp, Homo sapiens
acccttggttccaatggaaccaacaygtaaagcaagatcaactcagtaata

SEQ ID NO: 16 - DNA, 51 bp, Homo sapiens
aaataagtttttgatgtcttctataratatttcaacagtcttacaaggtat

SEQ ID NO: 17 - DNA, 51 bp, Homo sapiens
acacatatatgtgagtatgaaaatcrgacgtacttttttctaaactgaagc

SEQ ID NO: 18 - DNA, 51 bp, Homo sapiens
ctaactgttgtgttttaagagcggtmgtggcatgtccctagaccccctttt

SEQ ID NO: 19 - DNA, 51 bp, Homo sapiens
ttgcctatcaagtcttctgttttcaygctcttatgtcctctctcttctcga

SEQ ID NO: 20 - DNA, 51 bp, Homo sapiens
tgataatcccttccccgactaaagtkaatgtaaagagatttagtaacatag

SEQ ID NO: 21 - DNA, 51 bp, Homo sapiens
tcagtaatcttctgaaactactgtcsttgttgttcaaaaagcaatgaagcc

SEQ ID NO: 22 - DNA, 51 bp, Homo sapiens
tacaagtacatatcatattatgtgamccagagacctatctaaatgtataga

SEQ ID NO: 23 - DNA, 51 bp, Homo sapiens
aagaaaaggaaaatagccagaggtargtgaaatcaaatgagagaagtctaa

SEQ ID NO: 24 - DNA, 51 bp, Homo sapiens
actaagaatatcctgagttagtagaygtattagagttctctagagaaatag

SEQ ID NO: 25 - DNA, 51 bp, Homo sapiens
ctggaagccatggtagctttggtagmcatattactgaaatatagcccaaaa

SEQ ID NO: 26 - DNA, 51 bp, Homo sapiens
gaagaaaaagaagagctttcataagsagaaatgagtgtgagaggggaggta

SEQ ID NO: 27 - DNA, 51 bp, Homo sapiens
cagaaaatccaaggcagacataatgrattaatacgcacagatggagctgtt

SEQ ID NO: 28 - DNA, 51 bp, Homo sapiens
atctaggacagcctataccttgcacmaagaatgctaccaaattccaaccag

SEQ ID NO: 29 - DNA, 51 bp, Homo sapiens
atgtccttagcacagtgtccagcacrctacatatgcacagctattaatatt

SEQ ID NO: 30 - DNA, 51 bp, Homo sapiens
cataatttgtaatttaacaagtgacrgaatattttgacattagtaaatgaa

SEQ ID NO: 31 - DNA, 51 bp, Homo sapiens
tgagagagagtgattctacaaagatmagatcatgcagggtgttgctgaaga

SEQ ID NO: 32 - DNA, 51 bp, Homo sapiens
atttaccctaccacctcacagtaaasgttgctagaaagactgtcacaatac

SEQ ID NO: 33 - DNA, 51 bp, Homo sapiens
ataaaaatgaaatcaccagggagaaragcaattgactgagtttccatagtc

SEQ ID NO: 34 - DNA, 51 bp, Homo sapiens
ggagttctctggggctacaatttgarctatgaattattctgtgggaggaag

SEQ ID NO: 35 - DNA, 51 bp, Homo sapiens
gcatcagaacatattggtcaaaggastaaggctgtggtacaacatggccag

SEQ ID NO: 36 - DNA, 51 bp, Homo sapiens
tacttaacagatcctcacttggacaygaactatagaggccagtaaatggaa

SEQ ID NO: 37 - DNA, 51 bp, Homo sapiens
ctagggatttaggcttccacattcartctatagcctatagttattcatttt

SEQ ID NO: 38 - DNA, 51 bp, Homo sapiens
tttagtctcctaattcacctcatcaygtaaaagcaactctctaagtttgtt

SEQ ID NO: 39 - DNA, 51 bp, Homo sapiens
gcacatctgccatctttggtcagcarccctccaggactgccttcttcacac

SEQ ID NO: 40 - DNA, 51 bp, Homo sapiens
gcagttcaagatgagttggacacaaycaagaaaaatagggaacaaatatgt

SEQ ID NO: 41 - DNA, 51 bp, Homo sapiens
ttagtatcagtgactctctatggcasgatattttttatcagagcaggggaa

SEQ ID NO: 42 - DNA, 51 bp, Homo sapiens
agaggagtcattgagtgtttcttcartagagttaacaaatgtcccagtgtg

SEQ ID NO: 43 - DNA, 51 bp, Homo sapiens
ccaagctagagacaaaattggccacygaaacatatttttctgaaatgtaat

SEQ ID NO: 44 - DNA, 51 bp, Homo sapiens
ttaatattttcttcctatcgtgttcyaggattaaattgtatagtagccctc

SEQ ID NO: 45 - DNA, 51 bp, Homo sapiens
tatttaactttaagtagtacagtaawccaataattggggaaattaggatgt

SEQ ID NO: 46 - DNA, 51 bp, Homo sapiens
gctgtagatgtttgaccaagatgaaraaatagaaaatataagcaacatcga

SEQ ID NO: 47 - DNA, 51 bp, Homo sapiens
aaggtttttttaaatgataactttargaaggtgatatacaaaacttatcaa

SEQ ID NO: 48 - DNA, 51 bp, Homo sapiens
ataatatacaatgtgtgttttcagartccaccagcatagaggaaacaatca

SEQ ID NO: 49 - DNA, 51 bp, Homo sapiens
gccatctggtggtactttgaaacgasattgatttaatttttggacactgtg

SEQ ID NO: 50 - DNA, 51 bp, Homo sapiens
gggtagaaatagttgttatagatacscagaaaactgaggcaatgaaaaacc

SEQ ID NO: 51 - DNA, 51 bp, Homo sapiens
aagatgtctgccatggaggtctccartgccagtttcacctatacctctgac

SEQ ID NO: 52 - DNA, 51 bp, Homo sapiens
aaatgactggatattactgcacagtsaatctagtacgtgaacaagaagatt

SEQ ID NO: 53 - DNA, 51 bp, Homo sapiens
tgggccaactccagtttatctttaaycaaagctgtgctgacttaaatagtt

SEQ ID NO: 54 - DNA, 51 bp, Homo sapiens
attttatggtttaattccttgcattyaaattttctgatgcatctgggattt

SEQ ID NO: 55 - DNA, 51 bp, Homo sapiens
ggaaaggcctggagatagcccaacartatcctagtttgcaaagtaaaagaa

SEQ ID NO: 56 - DNA, 51 bp, Homo sapiens
ccttattcatcaccctcattctctcyaggctggtacatcccggtctaggaa

SEQ ID NO: 57 - DNA, 51 bp, Homo sapiens
ttgatttcctgggactcaagtctcayaggaaactgattaccataactcatt

SEQ ID NO: 58 - DNA, 51 bp, Homo sapiens
aagtctttttagttgattgttttgayatctataggcatacttaattcagga

SEQ ID NO: 59 - DNA, 51 bp, Homo sapiens
atcacgaacgaagcaaacttcagaaytgattaggtgaccttatatgtgtac

SEQ ID NO: 60 - DNA, 51 bp, Homo sapiens
attccactggatagaatcttccagartaacatgcaggaaagaaaattatta

SEQ ID NO: 61 - DNA, 51 bp, Homo sapiens
attccattaccacttcattaaaaaayatgctgtccccggtgctgaaactgc

SEQ ID NO: 62 - DNA, 51 bp, Homo sapiens
gcatttaaggtttgacttcctttctyaacttgtgaattctttatgttgtag

SEQ ID NO: 63 - DNA, 51 bp, Homo sapiens
tgagttacttatcatttaagaaatawccaaactgctaacattgtaaaagca

SEQ ID NO: 64 - DNA, 51 bp, Homo sapiens
ccaatattccctcattggactaaagrattaaagagcaaaataagtatgctc

SEQ ID NO: 65 - DNA, 51 bp, Homo sapiens
agctgaaggaataccttcagatcaaraaatatgatgtaaatatgcaggaca

SEQ ID NO: 66 - DNA, 51 bp, Homo sapiens
gcagattgccacatatctattacagracatcattgcctaaattaggcactc

SEQ ID NO: 67 - DNA, 51 bp, Homo sapiens
aagtgacagcaggtatgaatttaaaragaatggacattgaaaaagaacaaa

SEQ ID NO: 68 - DNA, 51 bp, Homo sapiens
tatgtgcttggtatagttcctggtayagagctattagtgtttatcctagga

SEQ ID NO: 69 - DNA, 51 bp, Homo sapiens
taataatggacaattacagaataacrcaatgcagaactgcaaatgacctta

SEQ ID NO: 70 - DNA, 51 bp, Homo sapiens
taaaaggcgcctaagatcattttaaracagcatttcaaagagttttcaaaa

SEQ ID NO: 71 - DNA, 51 bp, Homo sapiens
agctacctctgtaccctcaaaattayagcatcacattttaaacctacagtc

SEQ ID NO: 72 - DNA, 51 bp, Homo sapiens
aatcagtattgtaatggggaaggtayggagcattcaactatagagagacat

SEQ ID NO: 73 - DNA, 51 bp, Homo sapiens
tctactttgtcacacgttaactccarataaccacatgtagctgacctcatg

SEQ ID NO: 74 - DNA, 51 bp, Homo sapiens
gatagaggtttggagccagacagacsaaataaaaatctcagccacttagta

SEQ ID NO: 75 - DNA, 51 bp, Homo sapiens
aagtaaaaggaaacctgactatagakaattctgtgcctcccttctctgagg

SEQ ID NO: 76 - DNA, 51 bp, Homo sapiens
tatgaacccaacatactcaactcaayggctttcttttacaataacccttta

SEQ ID NO: 77 - DNA, 51 bp, Homo sapiens
agagagaaaaaaagcattttctttayggtttacaaagtgctttcatatgat

SEQ ID NO: 78 - DNA, 51 bp, Homo sapiens
ctgcttatctcagggaagccaaagaygactttacgaaagaggagattcagg

SEQ ID NO: 79 - DNA, 51 bp, Homo sapiens
ctgtgctcactgtgttcaataactcrgaaggagactgatgcaggcaagcat

SEQ ID NO: 80 - DNA, 51 bp, Homo sapiens
tgagtagctgagcttccataaatcaygtttcataacttggagctgcagagg

SEQ ID NO: 81 - DNA, 51 bp, Homo sapiens
tgtgaattatagggtcactttatgargatcaaggaaatagatttcttaaca

SEQ ID NO: 82 - DNA, 51 bp, Homo sapiens
gaatacagtaatttttagtataatgyaaattcgtagacaagattattgtaa

SEQ ID NO: 83 - DNA, 51 bp, Homo sapiens
catttggaatctactttcaatatcaygctcaggccaggcatggtggctcat

SEQ ID NO: 84 - DNA, 51 bp, Homo sapiens
tttcactcttttaaatcccagagccratacttgaacagaagtaagtttgct

SEQ ID NO: 85 - DNA, 51 bp, Homo sapiens
tgtggagagatgtaaaacaagaaacrcagtatattagcatagatgtatcag

SEQ ID NO: 86 - DNA, 51 bp, Homo sapiens
acaacttgctctggttgctaataagwcactatgaattaaggttttgacagc

SEQ ID NO: 87 - DNA, 51 bp, Homo sapiens
cacattcaacttcaaagctatatcaygggcctttgattttagttcctattg

SEQ ID NO: 88 - DNA, 51 bp, Homo sapiens
tggttatttaagggtgtgtaatagamtcctactccaacaggaatttggatg

SEQ ID NO: 89 - DNA, 51 bp, Homo sapiens
agacaaattaatgtaatgtaaggaayatagcctagtcagcaagacttaaat

SEQ ID NO: 90 - DNA, 51 bp, Homo sapiens
tgataggaatttgctgactagaagawtatcagctgacagcactaaatgaaa

SEQ ID NO: 91 - DNA, 51 bp, Homo sapiens
acagtcaattccacttgcttgcaaasgaaaaaccaccactgtaacgcagca

SEQ ID NO: 92 - DNA, 51 bp, Homo sapiens
tcattgagaatccaatatatactacycagtggaaatggggagtaaaaacta

SEQ ID NO: 93 - DNA, 51 bp, Homo sapiens
agctattattagattcaaaattgccsgattctaatttatattttaagtgta

SEQ ID NO: 94 - DNA, 51 bp, Homo sapiens
aactctgattacatttatttcaaccragagatgaaggaaacaaaggtccag

SEQ ID NO: 95 - DNA, 51 bp, Homo sapiens
agtgacatgagagttggtctttcaaytgggcgagattaatggctgtgggct

SEQ ID NO: 96 - DNA, 51 bp, Homo sapiens
ttcagatgtgtgtgctgacaggaaaygaagacactttcttctcagacagtg

SEQ ID NO: 97 - DNA, 51 bp, Homo sapiens
cttaaaatctagaacctgtaactcartaagatgtcagtcacaatgtgtccg

SEQ ID NO: 98 - DNA, 51 bp, Homo sapiens
ttttgttagttttgattaaagggaaygaggagctctgagtagggcagcaaa

SEQ ID NO: 99 - DNA, 51 bp, Homo sapiens
actcaggtcaattaaattagaatgamgatggagtaagtgcacgtactatga

SEQ ID NO: 100 - DNA, 51 bp, Homo sapiens
ctagaccaagccctctaaaacacaawtacacagggagtacaagaaggggac

SEQ ID NO: 101 - DNA, 51 bp, Homo sapiens
gccttttgtcattccacttggaaagrcaatttagtgctatgttttttgaac

SEQ ID NO: 102 - DNA, 51 bp, Homo sapiens
tctttcagggaaataatgattgaaartatcttacgttagattctaagaact

SEQ ID NO: 103 - DNA, 51 bp, Homo sapiens
cctaggatttcagcatctgttcttaygtgacgagaagaaacagctcccttg

SEQ ID NO: 104 - DNA, 51 bp, Homo sapiens
attgctcccaactgataccaacaccyaatattatatctttttttttctaaa

SEQ ID NO: 105 - DNA, 51 bp, Homo sapiens
tcacagcagttaccatactctattayactctctttactttcttatgtcctc

SEQ ID NO: 106 - DNA, 51 bp, Homo sapiens
tgctattaagttttattatttttaarcatctgtgatcaatcaaataggtaa

SEQ ID NO: 107 - DNA, 51 bp, Homo sapiens
cctgtgggtacagtcagctttccacrcagaggtactgtttcccgggaagga

SEQ ID NO: 108 - DNA, 51 bp, Homo sapiens
tccacaaatactaaaattgcaaaaaygattttggtgagaagtggcgaggag

SEQ ID NO: 109 - DNA, 51 bp, Homo sapiens
atgataggatttaccaaattatgtgraaatatatactgtcacctcccactt

SEQ ID NO: 110 - DNA, 51 bp, Homo sapiens
gtctcaccctagccctctactttgawaaggcagactgcaagtggcaatagt

SEQ ID NO: 111 - DNA, 51 bp, Homo sapiens
tattctatggttcggtatgttgggcyctttaagaaatggattctagagcct

SEQ ID NO: 112 - DNA, 51 bp, Homo sapiens
catttccctttagtcacactgtttcraactctggaaattccttccatctca

SEQ ID NO: 113 - DNA, 51 bp, Homo sapiens
ccattcgtcatcttttagtttcacayggttccattggttccagtcactgag

SEQ ID NO: 114 - DNA, 51 bp, Homo sapiens
atgcataatggtagttatattgttgwagaccattagaaaaatatggtatag

SEQ ID NO: 115 - DNA, 51 bp, Homo sapiens
tacccatgggacagatggcatttaawctaggccttgaagcaagggtggctc

SEQ ID NO: 116 - DNA, 51 bp, Homo sapiens
ttcctttacttctccatgtaaataaycatactagtaaatttataggaggcc

SEQ ID NO: 117 - DNA, 51 bp, Homo sapiens
acagattacggttttgagatttatartacaacctggggcaatgaagaaaaa

SEQ ID NO: 118 - DNA, 51 bp, Homo sapiens
tatgtcttaacaattttaatcagaaytcatatgacgtgaacagcatttttt

SEQ ID NO: 119 - DNA, 51 bp, Homo sapiens
ttgtttctaccattgcctggaaacartaaataatattcactactggttttc

SEQ ID NO: 120 - DNA, 51 bp, Homo sapiens
taaaattggacctttttagttatcayagaagtctattcagaaagagggtat

SEQ ID NO: 121 - DNA, 51 bp, Homo sapiens
ggtagaaaatatacagtttaagtaayaggaatgcatccatgagatatcagc

SEQ ID NO: 122 - DNA, 51 bp, Homo sapiens
ggttgggtataagatcctaagactayaagaaatgaaattttatatgggaga

SEQ ID NO: 123 - DNA, 51 bp, Homo sapiens
tcaaataaaagtgcatttctaaatgraactgattagactaggttctttgta

SEQ ID NO: 124 - DNA, 51 bp, Homo sapiens
gaataagaggccaattaaactagagwaagggatcttggtaggaaaatttta

SEQ ID NO: 125 - DNA, 51 bp, Homo sapiens
tttctgcaccatcggtagtttctacraggatggaagaacaataacaataat

SEQ ID NO: 126 - DNA, 51 bp, Homo sapiens
gcgagtttaagggatacagcagtaaytggaagaaccaaagaagacagggaa

SEQ ID NO: 127 - DNA, 51 bp, Homo sapiens
gataaaaatattatccaaaatgaacrcaagccaggctgacatttcactggg

SEQ ID NO: 128 - DNA, 51 bp, Homo sapiens
ctcattctactgtataaaaagtcaaygaactgaagagcaaatattaaagga

SEQ ID NO: 129 - DNA, 51 bp, Homo sapiens
ccatgggtcccaacgtgcccaggtamattcccagacaactgttgtcaatag

SEQ ID NO: 130 - DNA, 51 bp, Homo sapiens
catatcatgcagcaccctcgtttaayagtgattacagcagtttcacccagc

SEQ ID NO: 131 - DNA, 51 bp, Homo sapiens
ataactgcccacagcctgctcaaaaytttatttcacagcctgggtgcctga

SEQ ID NO: 132 - DNA, 51 bp, Homo sapiens
aatttggtattaatatggatctaaawgcaagaccctttcatttatatcaac

SEQ ID NO: 133 - DNA, 51 bp, Homo sapiens
aatgatatagtgtcaaaaataataaygatgatttgctctgctaaaagttga

SEQ ID NO: 134 - DNA, 51 bp, Homo sapiens
acttaatgggaaataagtatagatayagcaagtgccattataacaaagtta

SEQ ID NO: 135 - DNA, 51 bp, Homo sapiens
aacagtaatagtaataacagcaatarcagtgtgtccttgaggaagtaagct

SEQ ID NO: 136 - DNA, 51 bp, Homo sapiens
ccattgtggcaatagtctgaattgaygttcacaaattacttgaaggccaga

SEQ ID NO: 137 - DNA, 51 bp, Homo sapiens
attttcaacttactgttctaacttayattttgaattagaacattttaacta

SEQ ID NO: 138 - DNA, 51 bp, Homo sapiens
taatcagaagaacacattggaattamcagggaagcattttaaaaactcaca

SEQ ID NO: 139 - DNA, 51 bp, Homo sapiens
tcatgatggtatggtttgatgagtaytaaataggatattttgatattttat

SEQ ID NO: 140 - DNA, 51 bp, Homo sapiens
attgttttaaataactaatgaataawccataaactatcttctattaatgct

SEQ ID NO: 141 - DNA, 51 bp, Homo sapiens
tagaattcacagtattttcaagttaraggtacatgtaataaggaagttgtt

SEQ ID NO: 142 - DNA, 51 bp, Homo sapiens
gtcacaaacttgggaaaagctcgtakatttgaggcagctgtccagataata

SEQ ID NO: 143 - DNA, 51 bp, Homo sapiens
tcttcagaggtatatattattcagargtatgtatttttctttggaggtata

SEQ ID NO: 144 - DNA, 51 bp, Homo sapiens
ataggaattgaggtcaaaaaatgggyatgattaaagcagtattcaaagtgg

SEQ ID NO: 145 - DNA, 51 bp, Homo sapiens
ggtagcaagctgtatttgttcttccygcagtaatttatagtcccctaattt

SEQ ID NO: 146 - DNA, 51 bp, Homo sapiens
aaataatataacttagcctccttgawtattaaattactttgagtaaattac

SEQ ID NO: 147 - DNA, 51 bp, Homo sapiens
aaatggaaataattcattctattcckatttagaaaacattctagaaagtgg

SEQ ID NO: 148 - DNA, 51 bp, Homo sapiens
aagaagcaacagacgaagcatctaaycttgtgaataaaatgattgatttag

SEQ ID NO: 149 - DNA, 51 bp, Homo sapiens
agcatcttgtatgcagtgagtgttaygaattttctttttgttaaaatgaat

SEQ ID NO: 150 - DNA, 51 bp, Homo sapiens
caaatgaacaagttcagaattattasagtgttacagttgactccgcatcat

SEQ ID NO: 151 - DNA, 51 bp, Homo sapiens
tcttgtgaggaaacagatatgcaaaragataattattactgcagtatagaa

SEQ ID NO: 152 - DNA, 51 bp, Homo sapiens
cttaggtttgattgcttaccttaaawtctaacctcagctttggtttttcag

SEQ ID NO: 153 - DNA, 51 bp, Homo sapiens
ccaaaatctttaaagactttcaatarcttacaacctgtgttcatatattct

SEQ ID NO: 154 - DNA, 51 bp, Homo sapiens
tccatattgacttgaaagtagctaaytacagccaggcagagggactatcta

SEQ ID NO: 155 - DNA, 51 bp, Homo sapiens
ccaaggactgcaggtgagcccggcasagtgaagttcaaggacattgtccat

SEQ ID NO: 156 - DNA, 51 bp, Homo sapiens
cgtgctttggaaaaacacagtaagartcccttcacattgaaactagactga

SEQ ID NO: 157 - DNA, 51 bp, Homo sapiens
ttataatgaagaggtttcagaggaaktacatgtatcccttaaaagtgcttt

SEQ ID NO: 158 - DNA, 51 bp, Homo sapiens
tatatggtcaatctctggaacattckcagatttaacagccgagtcctggca

SEQ ID NO: 159 - DNA, 51 bp, Homo sapiens
tagtcaggctttgcacatgaactagrctagagctagagtaaccatcttatc

SEQ ID NO: 160 - DNA, 51 bp, Homo sapiens
tcttggggggttcaaagtcatgcaarggatttggtataaattaagatggca

SEQ ID NO: 161 - DNA, 51 bp, Homo sapiens
ttgtgcttgctttggccaagaatcasataagtagtaccatcctgggattat

SEQ ID NO: 162 - DNA, 51 bp, Homo sapiens
cagttcaaacatattaaaaatgctayggtaacaaattatcattgaagtttg

SEQ ID NO: 163 - DNA, 51 bp, Homo sapiens
aagaaggggtatcaggagaaaatcaytgaatatggaaaaacgaaaggacca

SEQ ID NO: 164 - DNA, 51 bp, Homo sapiens
gaatgggtctttcagtttttgagaaycagcaaaagagtgttgttgtagcaa

SEQ ID NO: 165 - DNA, 51 bp, Homo sapiens
aagggcaagcttttgcaggacagcargctagcctaatggaaacagattttt

SEQ ID NO: 166 - DNA, 51 bp, Homo sapiens
tcatctaattacagtaactgctcaasagtaatgtggaacaaatgcagtgcc

SEQ ID NO: 167 - DNA, 51 bp, Homo sapiens
agaagagttattgttttaaacctcargatatatttaggaagacatttgagc

SEQ ID NO: 168 - DNA, 51 bp, Homo sapiens
caccaggtgctgtttgcacagaatcyaattttggtgtatttaattcattag

SEQ ID NO: 169 - DNA, 51 bp, Homo sapiens
ctctcaatgatattatcacaaatcartctggtaggtattatgcacatcaga

SEQ ID NO: 170 - DNA, 51 bp, Homo sapiens
atgggctggagtgtgaggataatcargatggtactgtttgtacctccgtgg

SEQ ID NO: 171 - DNA, 51 bp, Homo sapiens
ctagcccttaatgttttcaagaatcwaatagcattcagtgcaatcatacaa

SEQ ID NO: 172 - DNA, 51 bp, Homo sapiens
cgtggcattacctccagcaacagtcrataacaggattatttttggaaaaaa

SEQ ID NO: 173 - DNA, 51 bp, Homo sapiens
ttctacttatggtatgttaccacctyacaatacaaaggaatataacaataa

SEQ ID NO: 174 - DNA, 51 bp, Homo sapiens
acaactgccaggaactagccaattarctgtcaatctatctatgttcataca

SEQ ID NO: 175 - DNA, 51 bp, Homo sapiens
aacgttttatataagcaggactcaarttagttaagagagtttattatgtaa

SEQ ID NO: 176 - DNA, 51 bp, Homo sapiens
atgaatctatctcctgaatcaaatayactgcaaaggagaattttcaacttg

SEQ ID NO: 177 - DNA, 51 bp, Homo sapiens
accactttattagacttacagcaaaygaaagaaacgtctaactttaatgtg

SEQ ID NO: 178 - DNA, 51 bp, Homo sapiens
gtggagggattggagccagctgaagycagagttgtgaagttctgcttagaa

SEQ ID NO: 179 - DNA, 51 bp, Homo sapiens
caaggtcatgccagaaaattaattcmaatcaaatcatgagccagatcaaca

SEQ ID NO: 180 - DNA, 51 bp, Homo sapiens
aaccttcaaggcaggagagttaggcraatacagcatctttgaacactttat

SEQ ID NO: 181 - DNA, 51 bp, Homo sapiens
atttatcttgctcttttatggacctsaggatgttcaaaaggacttttcttt

SEQ ID NO: 182 - DNA, 51 bp, Homo sapiens
gaagcacagtccatcaattgtttcayggaaaagctagcaaaagaattagcc

SEQ ID NO: 183 - DNA, 51 bp, Homo sapiens
aatactggtccttaggtgaaagataygacacaattagtaagtgctgtctag

SEQ ID NO: 184 - DNA, 51 bp, Homo sapiens
ctcaggaggaatacatagttatggaygatacattctgagcaaatgagagtc

SEQ ID NO: 185 - DNA, 51 bp, Homo sapiens
ataaattggaaaggcagagtgttccrgagataaggagtcagactttggagt

SEQ ID NO: 186 - DNA, 51 bp, Homo sapiens
ctacatatttctttctgattccttgyacttctatcccaaaagagtgtgcct

SEQ ID NO: 187 - DNA, 51 bp, Homo sapiens
caattacattatgagatactagcaakaattcctaaggataatgttctcctt

SEQ ID NO: 188 - DNA, 51 bp, Homo sapiens
tggcgagaggcaccatcagaattgawctcaggtctatgtggcagccagcct

SEQ ID NO: 189 - DNA, 51 bp, Homo sapiens
gctgaactccagtcatgaaccgtcasctagctaatatctatcttctgtgag

SEQ ID NO: 190 - DNA, 51 bp, Homo sapiens
aattatgattttgagaagtggcccakcgagggttttgtcttttcattatta

SEQ ID NO: 191 - DNA, 51 bp, Homo sapiens
tttagtagctctgggtgcaaatgtaygatccaccattccgtgtgctactgt

SEQ ID NO: 192 - DNA, 51 bp, Homo sapiens
gggccacaaggatctgtgagaacccrgcctatcctcaaagaggccaccatc

SEQ ID NO: 193 - DNA, 51 bp, Homo sapiens
caaaaactggtggaaagaatgcgaayggtataatgggaatgtttctaacac

SEQ ID NO: 194 - DNA, 51 bp, Homo sapiens
aatagtttctgataatttaatcccastagagaaaacactcatttacatatc

SEQ ID NO: 195 - DNA, 51 bp, Homo sapiens
ttttcttatatttaccacaaatcacrgataattctcaagtgttcacagtct

SEQ ID NO: 196 - DNA, 51 bp, Homo sapiens
gattgcaggaaggttgtaactttgayaatatcctttaccttgatttattct

SEQ ID NO: 197 - DNA, 51 bp, Homo sapiens
tgttgtggcatatgccaggacttaaytgtagccataatttcatgttgagcc

SEQ ID NO: 198 - DNA, 51 bp, Homo sapiens
atggattgaacattatatacacaagraattttacccactgtattgcattta

SEQ ID NO: 199 - DNA, 51 bp, Homo sapiens
acaatccaattaaatgaatagttagrattactccttgaacataatagcaaa

SEQ ID NO: 200 - DNA, 51 bp, Homo sapiens
ccccgttgttctcagcataaaggggwccaagtacttaaataaaaaattcat

SEQ ID NO: 201 - DNA, 51 bp, Homo sapiens
tatttttctgaaaaacactcgctcaygttcaccctaatataaccttattgg

SEQ ID NO: 202 - DNA, 51 bp, Homo sapiens
ctgctcactttttattcattcttcamgacttaagtcaggattttccccagg

SEQ ID NO: 203 - DNA, 51 bp, Homo sapiens
ggctgaagtagactagaaagaaaaawattgagtcaatgttgtatagaggca

SEQ ID NO: 204 - DNA, 51 bp, Homo sapiens
aataaaccttgcaattgaggtattcrcagtgtccacaggaacaaactcatt

SEQ ID NO: 205 - DNA, 51 bp, Homo sapiens
ataccttcattacctcacgagtgcartgatgtgaagatgtctcaattctca

SEQ ID NO: 206 - DNA, 51 bp, Homo sapiens
gcagtgtcaaggccttgggatctccractcaatttctgtcttttctgtcaa

SEQ ID NO: 207 - DNA, 51 bp, Homo sapiens
atgactctatacaacctttctttgaygcacttgagcttttgtaataatccc

SEQ ID NO: 208 - DNA, 51 bp, Homo sapiens
ctcggactcctcatcttgaccagagycattgtctttggttaaataaccaaa

SEQ ID NO: 209 - DNA, 51 bp, Homo sapiens
caagggaaaaatgtcaagaatcaaaraatgatcaaccctgtcataccacag

SEQ ID NO: 210 - DNA, 51 bp, Homo sapiens
ccatcagattcagtcaatctcaatayattaggtttaacattctaaaactca

SEQ ID NO: 211 - DNA, 51 bp, Homo sapiens
aggttaaagggtatatctcacatacrcttcttcttttctgacatttcaaac

SEQ ID NO: 212 - DNA, 51 bp, Homo sapiens
agataatgatgttccagattctaggwcagtcttgtggtatgtttcacctca

SEQ ID NO: 213 - DNA, 51 bp, Homo sapiens
ctggactctaattgctgcatgtaaasaaacatcatctggaacagagccacc

SEQ ID NO: 214 - DNA, 51 bp, Homo sapiens
cgcatgcacacacacacacatgctamagaaggacaaagcacagatggacag

SEQ ID NO: 215 - DNA, 51 bp, Homo sapiens
atgccatttttctttgatgcaatcaygaagaaaagtggaaatgcaggacga

SEQ ID NO: 216 - DNA, 51 bp, Homo sapiens
ataaggaaaatgtgttgtcatcaatragaaaaaatctaatgagtcaatttc

SEQ ID NO: 217 - DNA, 51 bp, Homo sapiens
aatagccccatgcctcagtacttctyaaaccatgtcattgttctaaagcct

SEQ ID NO: 218 - DNA, 51 bp, Homo sapiens
ttaatatatataattctgggtgaacyagtgtactatattacaagcttttct

SEQ ID NO: 219 - DNA, 51 bp, Homo sapiens
tcaagctcttaaaatagggcccacasagttacatccaatctcctctactga

SEQ ID NO: 220 - DNA, 51 bp, Homo sapiens
cgagacttttcttaatgcaccaagckatttcattctttctcattcattcat

SEQ ID NO: 221 - DNA, 51 bp, Homo sapiens
ttgcaatgccaccactcagttctaayacaccattcctaagcctccaacaat

SEQ ID NO: 222 - DNA, 51 bp, Homo sapiens
gttatgtgttggatctggccaaaagyatatctcacaccaaatatatgatgt

SEQ ID NO: 223 - DNA, 51 bp, Homo sapiens
tatggagcaggctcctgggatactakagtcattatgtaacacatttttttt

SEQ ID NO: 224 - DNA, 51 bp, Homo sapiens
gggaaataagttttcaatagcactakacttaggcttaactcacgttattaa

SEQ ID NO: 225 - DNA, 51 bp, Homo sapiens
gtcgtaatcaacctaagggcataaaycttgaatcccaactcagggaaaaga

SEQ ID NO: 226 - DNA, 51 bp, Homo sapiens
ttagttattttgctatttccttttcyaagcaatttgaggtgatcacatcat

SEQ ID NO: 227 - DNA, 51 bp, Homo sapiens
cctgctctttagtgtatctggcgaargttatcatattttcttaacgagatg

SEQ ID NO: 228 - DNA, 51 bp, Homo sapiens
agagaccccaaatcaaatcaggagckattaaatgactcattcaattacagg

SEQ ID NO: 229 - DNA, 51 bp, Homo sapiens
cacgttttctttcatcttgtgtttgsacattagccacagagggattgatgt

SEQ ID NO: 230 - DNA, 51 bp, Homo sapiens
ctagacaagaaaagagactttataastggattatcttgctttccagtgtta

SEQ ID NO: 231 - DNA, 51 bp, Homo sapiens
ggatacgtttttaaacctgatgacaragacagagctgaagggcttcatcgt

SEQ ID NO: 232 - DNA, 51 bp, Homo sapiens
aaaattaacaaatgagacaataatasgaaatcatgaagggctgcaattaca

SEQ ID NO: 233 - DNA, 51 bp, Homo sapiens
tatttacagaacttgttcctgattaygtaagatttaactaccaagcatatg

SEQ ID NO: 234 - DNA, 51 bp, Homo sapiens
ctgtgtccctcctcactgggtttccrctgcacacttggttgcagtgaagga

SEQ ID NO: 235 - DNA, 51 bp, Homo sapiens
ataccaaaatacatagcacttaaaaytggtgttcagaatcaagcttcaaaa

SEQ ID NO: 236 - DNA, 51 bp, Homo sapiens
ccatgatttccttgttgacagtgagyacttaaacagacttctatgaccagc

SEQ ID NO: 237 - DNA, 51 bp, Homo sapiens
tgacttcagttttgaaattgatgaayctgcatacaacatatcccttaaaat

SEQ ID NO: 238 - DNA, 51 bp, Homo sapiens
taaaaaatcatttcttgcaggctaawcctaccataattatctacataaaat

SEQ ID NO: 239 - DNA, 51 bp, Homo sapiens
tcataagactctcagaccctgtataygtaaaaccagctatctagacagtgc

SEQ ID NO: 240 - DNA, 51 bp, Homo sapiens
ctttccttcaatgatccacaatactractagcaatctgagactgaaagtta

SEQ ID NO: 241 - DNA, 51 bp, Homo sapiens
tttatttgtgttgaattagcaattasctccgctcaataaatcctactctat

SEQ ID NO: 242 - DNA, 51 bp, Homo sapiens
catttgctttgtgaatgcttgatgaygctgttgtataatgactgaagtaaa

SEQ ID NO: 243 - DNA, 51 bp, Homo sapiens
tttctacaaccactgggtagtaaccrgaagctgactcctgacttgagtata

SEQ ID NO: 244 - DNA, 51 bp, Homo sapiens
atccaccattttgaaatctaattcasgctcaaaactatatctggtttgtgt

SEQ ID NO: 245 - DNA, 51 bp, Homo sapiens
gaaaattgaatgtgacaggtgttcartaaataattggttcaataaattggt

SEQ ID NO: 246 - DNA, 51 bp, Homo sapiens
gtgtacatggttaatataagcaagcygaaagttgaggtgatgctcccgaga

SEQ ID NO: 247 - DNA, 51 bp, Homo sapiens
tagcattcctatcaatagccttaacwctgttcctcacctgcattttaccat

SEQ ID NO: 248 - DNA, 51 bp, Homo sapiens
gaagtagtgacaagttcatattgaaragagtgaattagtgttcctttcaat

SEQ ID NO: 249 - DNA, 51 bp, Homo sapiens
gtttacaacaaaactataatgtatgwagcagtcataaaagaaactttatta

SEQ ID NO: 250 - DNA, 51 bp, Homo sapiens
gcgagctcatataaacatctcttacrcatgattctgatccttaaacattga

SEQ ID NO: 251 - DNA, 51 bp, Homo sapiens
ccataaatattccttggacacccacyatgaagacatactttattttcctcc

SEQ ID NO: 252 - DNA, 51 bp, Homo sapiens
cacagcagtgtgaggtagctattgtmaaggaaaatgaggctcataaaggtt

SEQ ID NO: 253 - DNA, 51 bp, Homo sapiens
gagcaaagcttggttagaatgtgtakgacactgattaatatactttcaaag

SEQ ID NO: 254 - DNA, 51 bp, Homo sapiens
taagtatggcatgtagcgtcttcaamagttcatggtggagggtaggagtac

SEQ ID NO: 255 - DNA, 51 bp, Homo sapiens
tgtgtggtccttttggaggcatttcycttttaccattcaccgccttcgtgg

SEQ ID NO: 256 - DNA, 51 bp, Homo sapiens
ttaaccgaatttatttttcttcaaamggctaattacactttaatatgttgc

SEQ ID NO: 257 - DNA, 51 bp, Homo sapiens
tccaaacacgtttgacacatctaaakaaccttctaaaggatgactaaacta

SEQ ID NO: 258 - DNA, 51 bp, Homo sapiens
gattgtaggaaaattcatacaaaaaratgctacataggaatataaactgaa

SEQ ID NO: 259 - DNA, 51 bp, Homo sapiens
tatagcagctatatgagtttggcaaygggcattaagctacatgagtttgca

SEQ ID NO: 260 - DNA, 51 bp, Homo sapiens
acatacagtatgtgctgagtacttakgaacttttggttatcctctattatt

SEQ ID NO: 261 - DNA, 51 bp, Homo sapiens
tccttcaatattgacacaataaagamagacagttggtatagctatctttct

SEQ ID NO: 262 - DNA, 51 bp, Homo sapiens
atttaggtcaagcaatcaagcacaaygattcaaaattgaaaccaacttttt

SEQ ID NO: 263 - DNA, 51 bp, Homo sapiens
cctccaacatatattcttatatgtcractgcctaatattccattgaagggc

SEQ ID NO: 264 - DNA, 51 bp, Homo sapiens
tgggtgaacttcaactaatacaaaasaagagacaagagaaaggtaggcagt

SEQ ID NO: 265 - DNA, 51 bp, Homo sapiens
gggccagagtctgaagagaaagacartgggttgcaaaggtggtgaatgaat

SEQ ID NO: 266 - DNA, 51 bp, Homo sapiens
ctttccacctgctttgcccattttcrcctggtggagtagaagctgtcagtt

SEQ ID NO: 267 - DNA, 51 bp, Homo sapiens
ttgctttactgtgagtgaattggggmagtttgcttatctagaagcagctta

SEQ ID NO: 268 - DNA, 51 bp, Homo sapiens
tttaaagggcaactctgattgcaggraagagattttattaaaaggaagaag

SEQ ID NO: 269 - DNA, 51 bp, Homo sapiens
gagaggagactggagatgtaaacacyagttggatcatggagggtctgtgag

SEQ ID NO: 270 - DNA, 51 bp, Homo sapiens
atgaggatgtcactaactgtcctaawcttaagcattgtcgaaaataaaatg

SEQ ID NO: 271 - DNA, 51 bp, Homo sapiens
ccaaccatcacctttaaggaattacrgataccctaagactctgggtgtttc

SEQ ID NO: 272 - DNA, 51 bp, Homo sapiens
aaatcagttactaaaactggctcaaytgatgcggagaacatttagaacaat

SEQ ID NO: 273 - DNA, 51 bp, Homo sapiens
gtggttctatgataaagactcttacraacgttgtgacaaaatatctaaaat

SEQ ID NO: 274 - DNA, 51 bp, Homo sapiens
tactaataaactcatatactcagtargctaatagaaaatgagcagaaatag

SEQ ID NO: 275 - DNA, 51 bp, Homo sapiens
accacaatattttacaatagcccaaygtagctgagttcagcatacctggat

SEQ ID NO: 276 - DNA, 51 bp, Homo sapiens
aagcaatataagaattcctgcataaygtctttgagaagaatattctccttg

SEQ ID NO: 277 - DNA, 51 bp, Homo sapiens
aaaatcactttaattaacacaagaaktattcaagtgaatgactaaagtgac

SEQ ID NO: 278 - DNA, 51 bp, Homo sapiens
aatcacatctggaagtgagcccatcragagagtgttctctacttcctacac

SEQ ID NO: 279 - DNA, 51 bp, Homo sapiens
caagtacaaagagtccctgacttacrgatgaaatggtgactgactgacttg

SEQ ID NO: 280 - DNA, 51 bp, Homo sapiens
ctgcagtgtgagcacagaggattaayggtagtcacagagagcctgagcagc

SEQ ID NO: 281 - DNA, 51 bp, Homo sapiens
aaagtagtagctgtatacacaatagwagttgctaactgaaatgatcttttt

SEQ ID NO: 282 - DNA, 51 bp, Homo sapiens
tattctcttcatttttaaaatggggrcatcatctttccttatgacatcttg

SEQ ID NO: 283 - DNA, 51 bp, Homo sapiens
atgtccctaggtgctatacatagaaygctgatttgctgtatcccaaatatg

SEQ ID NO: 284 - DNA, 51 bp, Homo sapiens
agaaatatttagcctattcttggatwaagtgctaattttagggagattcac

SEQ ID NO: 285 - DNA, 51 bp, Homo sapiens
tttaggctaacatctgactctattarcttctgaagagaagaaccataaata

SEQ ID NO: 286 - DNA, 51 bp, Homo sapiens
tgtgtcttgtgtcatgcagaccaagkatattgaaagaaagctactatgagg

SEQ ID NO: 287 - DNA, 51 bp, Homo sapiens
ttccagtgtatttgccagaaaatacrcttttacactttagcttaactggac

SEQ ID NO: 288 - DNA, 51 bp, Homo sapiens
tttaactcataaacataactttacaytgccctaaacaaacataactaaatt

SEQ ID NO: 289 - DNA, 51 bp, Homo sapiens
aaaaaagtgttagaagcaggagggaycttacaaataatctagttcagagat

SEQ ID NO: 290 - DNA, 51 bp, Homo sapiens
ttcagtacaacttaacagctagatayactccaaggtctttaatgtgtattt

SEQ ID NO: 291 - DNA, 51 bp, Homo sapiens
gcaagtccattatgtgacgtgcctcyatttcaaggatatgagatgtgttgg

SEQ ID NO: 292 - DNA, 51 bp, Homo sapiens
taaaagtctcccattcaactattcargtcattcagctctctagagatgcat

SEQ ID NO: 293 - DNA, 51 bp, Homo sapiens
atcttctttttttacaaattggttargtgaaacagagaaattaagtgactt

SEQ ID NO: 294 - DNA, 51 bp, Homo sapiens
gagtctgagattaagaaaattggaargtggtactcaaagagatagaagtta

SEQ ID NO: 295 - DNA, 51 bp, Homo sapiens
aagcagtacaatgaatgagcttatayagttatttcatcagaaataggcaca

SEQ ID NO: 296 - DNA, 51 bp, Homo sapiens
atcgtgttcaaggtttgctgttatayggacccgatgtcaagcttggagaaa

SEQ ID NO: 297 - DNA, 51 bp, Homo sapiens
ccacgtaaaagctagtccagaaaacyggtatgaaagtcagttggcttgcta

SEQ ID NO: 298 - DNA, 51 bp, Homo sapiens
cattgtgacatctgtaattactcaaygcactaatcctcataagtgtagcca

SEQ ID NO: 299 - DNA, 51 bp, Homo sapiens
attcctctgtgttcagaactttacawaataataacagtctgagacaaacaa

SEQ ID NO: 300 - DNA, 51 bp, Homo sapiens
tgttaaactctcattcttaactgtasatcttcttttcccagagtaatggaa

SEQ ID NO: 301 - DNA, 51 bp, Homo sapiens
tcttaaaagtgaatttgcagtatgayagatgaattagtggagaaagaatag

SEQ ID NO: 302 - DNA, 51 bp, Homo sapiens
tcactcccttagagctgttggacaamctttggctaataaagattaaacaaa

SEQ ID NO: 303 - DNA, 51 bp, Homo sapiens
cctgcataggctgactaaagtgctasggttctcataaactagtacataaag

SEQ ID NO: 304 - DNA, 51 bp, Homo sapiens
atcataaggatggaaagagcttggaragttctcacaacaagaggccagtgt

SEQ ID NO: 305 - DNA, 51 bp, Homo sapiens
taagaggccctagggtgatgctaaayggcaaattagatacatgtctgctgt

SEQ ID NO: 306 - DNA, 51 bp, Homo sapiens
agtggttgcctcactgaaaagaaacyggtagctaggaaacagggataggag

SEQ ID NO: 307 - DNA, 51 bp, Homo sapiens
tacaataaaatttgagaaccattgcyctataaagaaaaagaatgaatgttt

SEQ ID NO: 308 - DNA, 51 bp, Homo sapiens
cgcatctcacaaactaaggaccaaaktctgaaacgagttatctgcaaggaa

SEQ ID NO: 309 - DNA, 51 bp, Homo sapiens
agatccagcaggaggttagcgtgaargaatcacatcagatataggtaagaa

SEQ ID NO: 310 - DNA, 51 bp, Homo sapiens
tgaagcatttggttatggggtgaacrcaaatcaaatattgtgagatgtttc

SEQ ID NO: 311 - DNA, 51 bp, Homo sapiens
agtgaatatactagtggatcatcacraataataaactagtgactcaaacat

SEQ ID NO: 312 - DNA, 51 bp, Homo sapiens
tcagaaatatcgacttcctgtcctaygaaccaagagattgtgatttaatag

SEQ ID NO: 313 - DNA, 51 bp, Homo sapiens
tgagatcttatgtttagacactacgratcttttatttttcaatctcattgt

SEQ ID NO: 314 - DNA, 51 bp, Homo sapiens
aggaatcataaaacagccattgcaarttaaatgaatgatgtttagttattg

SEQ ID NO: 315 - DNA, 51 bp, Homo sapiens
tagagcattagagaaatctgctgaaygagagagacctggcattgggtctta

SEQ ID NO: 316 - DNA, 51 bp, Homo sapiens
aaggacatatcagctgtgtttataartagtgagactaactccacaaaccaa

SEQ ID NO: 317 - DNA, 51 bp, Homo sapiens
tccagagtctgctcttataacctcaygttacattatgctctccattgggtt

SEQ ID NO: 318 - DNA, 51 bp, Homo sapiens
aacctttttggttcacctgcataatyagtccaaagtaagcatacattattt

SEQ ID NO: 319 - DNA, 51 bp, Homo sapiens
catccattcaataaattgttacagarcacctaatatgggccagacgtgtca

SEQ ID NO: 320 - DNA, 51 bp, Homo sapiens
ggaaccacagatatcttttacccacrgagaaacattttcatcaaataataa

SEQ ID NO: 321 - DNA, 51 bp, Homo sapiens
atcccctgtatagctgaaactcccgrattggaagtggcagagccatctcta

SEQ ID NO: 322 - DNA, 51 bp, Homo sapiens
ttatcacatacaaatccagaaaataygacaaactccattattcacaactag

SEQ ID NO: 323 - DNA, 51 bp, Homo sapiens
tacattatctctcaagcaaggtgacrcttttttcaaggttaattatttata

SEQ ID NO: 324 - DNA, 51 bp, Homo sapiens
actcaagtccttcagctacgttcaartgagtgacctactcactcaaggcag

SEQ ID NO: 325 - DNA, 51 bp, Homo sapiens
ctaccttgttttaggtacagtgtcamaatattccttctcttattccatcca

SEQ ID NO: 326 - DNA, 51 bp, Homo sapiens
gccaggagctgaggagacggttatayaccaggaagttgagggagttccagc

SEQ ID NO: 327 - DNA, 51 bp, Homo sapiens
gagcatctgtctgggaaggttattcragtttaaggatgctgcagttacaat

SEQ ID NO: 328 - DNA, 51 bp, Homo sapiens
aaacttccttgtagtcaatgggagargtaaacctgaacaaaatgtgtgtgt

SEQ ID NO: 329 - DNA, 51 bp, Homo sapiens
aaaaatccgcctatctaggcttatgmatgttatataataccacagatatga

SEQ ID NO: 330 - DNA, 51 bp, Homo sapiens
acgcttatcaaaaagaactggggtamgttattcatcaaagttttatcatta

SEQ ID NO: 331 - DNA, 51 bp, Homo sapiens
gcaggtaccgccaaattcatcatggmacccatctgcattgtttgtgttttt

SEQ ID NO: 332 - DNA, 51 bp, Homo sapiens
ggaaatatgagtttgcaaagtataargtactgaggaaccttctctgtaaat

SEQ ID NO: 333 - DNA, 51 bp, Homo sapiens
tatggagtattgatcagtggcgggayacagtttgaaggtagagggagaggc

SEQ ID NO: 334 - DNA, 51 bp, Homo sapiens
ctccatggcacctcacagtaaacacrcattcataagggtcgcaggggcaaa

SEQ ID NO: 335 - DNA, 51 bp, Homo sapiens
tctaggagcaatgtcagggcaattascacttggtcagtttctagtgtttca

SEQ ID NO: 336 - DNA, 51 bp, Homo sapiens
cagagcagctgtgttgatcacctagraagtacaaactaaatgtggatcatg

SEQ ID NO: 337 - DNA, 51 bp, Homo sapiens
ccccatgactaatgactcttccacaytggacagcctttttagcagcctcca

SEQ ID NO: 338 - DNA, 51 bp, Homo sapiens
acaatggatatgattgaacaattcarctggagtatccaaaaacgtatgaca

SEQ ID NO: 339 - DNA, 51 bp, Homo sapiens
ggatttctgcctttgctacttgccaygtttgtgaagtctcctaccctttgt

SEQ ID NO: 340 - DNA, 51 bp, Homo sapiens
tcttactgtcattagcttccatcccrcgagctctctctggaattcactctt

SEQ ID NO: 341 - DNA, 51 bp, Homo sapiens
aagaaacatctacagggccgccgcasacgggtggtaagcgaaacttctaga

SEQ ID NO: 342 - DNA, 51 bp, Homo sapiens
tacggagcaaaaccatggctcatgcmaacaatatcccatctacacgcatac

SEQ ID NO: 343 - DNA, 51 bp, Homo sapiens
cgatccaactacataaaaatttgaaragcacaaagaacattacatcttggg

SEQ ID NO: 344 - DNA, 51 bp, Homo sapiens
tgcatgtgtatatgtaaaaatattaygtatcagtcaggttaagttgcattt

SEQ ID NO: 345 - DNA, 51 bp, Homo sapiens
tgacaaatgtgcgtcctgtgcatgarctgcgaagattttccatgatcagca

SEQ ID NO: 346 - DNA, 51 bp, Homo sapiens
caaattctggcttaaactgactaaayttacctgggtctccatgcaatgttt

SEQ ID NO: 347 - DNA, 51 bp, Homo sapiens
caaatgacataacagcatctaaaagyccaagtttgtaggatttgctgactt

SEQ ID NO: 348 - DNA, 51 bp, Homo sapiens
tgtgtaggtagagcaagtagccgctraagagtaatgttcatgaaacttaag

SEQ ID NO: 349 - DNA, 51 bp, Homo sapiens
cttgctaataatatgtacgccctaayagagtccatgcatctcttaaccaaa

SEQ ID NO: 350 - DNA, 51 bp, Homo sapiens
ttgtattcctgcttaatttttagacycatttggaagcagtgcatttttatc

SEQ ID NO: 351 - DNA, 51 bp, Homo sapiens
ctcagcggggcactcatacaagggcyaacttggaaagggacagataccagt

SEQ ID NO: 352 - DNA, 51 bp, Homo sapiens
taattaaaattctgtgcttcttagayggagacactctatgttttacaatga

SEQ ID NO: 353 - DNA, 51 bp, Homo sapiens
gcagctttgcccttccttcctcactragagccctatagcccaggacggtcc

SEQ ID NO: 354 - DNA, 51 bp, Homo sapiens
acatggtaggtatttgatacagagaygaatgaatgctcttcagttccccag

SEQ ID NO: 355 - DNA, 51 bp, Homo sapiens
aaaggctaaaatattttccacttgamtaaggcagtattaaaaacgaattag

SEQ ID NO: 356 - DNA, 51 bp, Homo sapiens
taaaaagtcacagtttagcccagaasgaggtatctccaagtttgtcagttc

SEQ ID NO: 357 - DNA, 51 bp, Homo sapiens
ctgtgactttttaaaatcacaattcratgaaaagttcaacacactaaaata

SEQ ID NO: 358 - DNA, 51 bp, Homo sapiens
gtcccattaattcattcaagagtccyaatgctaattctatcaaaaaaccca

SEQ ID NO: 359 - DNA, 51 bp, Homo sapiens
gtctacatattttcaaagtatttcaygacattcactcaactattcataaat

SEQ ID NO: 360 - DNA, 51 bp, Homo sapiens
gaattcaggaaagttaaatgtgtaartactgcggctcctgagaatattatg

SEQ ID NO: 361 - DNA, 51 bp, Homo sapiens
gcaatatcttaaaagagagataatargcagttgagattcaaccccacccca

SEQ ID NO: 362 - DNA, 51 bp, Homo sapiens
tgtaagcatcctgcactcatgatacragtcaccaaatcctgaaagcccata

SEQ ID NO: 363 - DNA, 51 bp, Homo sapiens
tcttgtctgcacagtgacatcagaaytcagttaaataattaaagactgtta

SEQ ID NO: 364 - DNA, 51 bp, Homo sapiens
gctcactgatcctttttgtttctaartgtcctaacagacttgcctactcca

SEQ ID NO: 365 - DNA, 51 bp, Homo sapiens
ccctgcattcttgtctgtttcctggratctataatgcccatgccctgtatc

SEQ ID NO: 366 - DNA, 51 bp, Homo sapiens
tccacactatgagatgctttgatggyaagatccatgttttgtgaagaccag

SEQ ID NO: 367 - DNA, 51 bp, Homo sapiens
gtatgtcatttctatgcctttgttayattcttgtgtaatttaaataagatg

SEQ ID NO: 368 - DNA, 51 bp, Homo sapiens
gaagcagagagaagaggccaaataayacgttggttcagctcaatccatgcc

SEQ ID NO: 369 - DNA, 51 bp, Homo sapiens
gcatagagttccaggatgtagaaacrgatttggttggaggagggaaataag

SEQ ID NO: 370 - DNA, 51 bp, Homo sapiens
gtgttatacaaggcatccatggtaargttctaagaagactgttggaggata

SEQ ID NO: 371 - DNA, 51 bp, Homo sapiens
aagaaacacccaacaataatgaaaayggtaggataagggcactccaaatga

SEQ ID NO: 372 - DNA, 51 bp, Homo sapiens
cacagcaaaagcctttctgataagcrgagatcaataccaggagaatatcta

SEQ ID NO: 373 - DNA, 51 bp, Homo sapiens
gtacagagtaatatggtgctaaaccygaattaaatttgtgactcggaaatt

SEQ ID NO: 374 - DNA, 51 bp, Homo sapiens
gctatggagacacttgcttcccatgyatgcttattaaatatataacccaac

SEQ ID NO: 375 - DNA, 51 bp, Homo sapiens
gtaactttgtaaactaatattctgtyaaacttggcgatttgttttttttga

SEQ ID NO: 376 - DNA, 51 bp, Homo sapiens
gccatgttatcttgttggttagcacrattatgcaaacaagttgctcagtga

SEQ ID NO: 377 - DNA, 51 bp, Homo sapiens
tctcttgtgctgtcatgaagggtacrgatctatcattgactccaagaccaa

SEQ ID NO: 378 - DNA, 51 bp, Homo sapiens
tagctcccaactggtatgatctgaaraatcagcggttactagggtaggaga

SEQ ID NO: 379 - DNA, 51 bp, Homo sapiens
tcttggttttgccagaaccctggtaygtaaattctctcctacttggtgaaa

SEQ ID NO: 380 - DNA, 51 bp, Homo sapiens
atgaagtctctattattgaaagaaayggatataagcaggtaaagctcaact

SEQ ID NO: 381 - DNA, 51 bp, Homo sapiens
agtatacattaagcaatgatcagacraatgaaaagctgtgtaatgaaacct

SEQ ID NO: 382 - DNA, 51 bp, Homo sapiens
gtcagtgagagagactgaagcataayaccgtaaattaataatgacctcatg

SEQ ID NO: 383 - DNA, 51 bp, Homo sapiens
atcatcttaaaattagcatcccttaygtattgatttgatgagaactcttta

SEQ ID NO: 384 - DNA, 51 bp, Homo sapiens
agactggagacaacaggaatggtaaygacttctcaagaggagttgggattt

SEQ ID NO: 385 - DNA, 51 bp, Homo sapiens
aagtacatcacaaatcacaaatgtaygacaagcctgcatgcgatgcttttg

SEQ ID NO: 386 - DNA, 51 bp, Homo sapiens
ggtctctaccactctctgaagataarcaataggcatttatttattcattcc

SEQ ID NO: 387 - DNA, 51 bp, Homo sapiens
ttttgtttgtttgtttgtttagcaartacattccttatatacatttatttg

SEQ ID NO: 388 - DNA, 51 bp, Homo sapiens
agccagtaaataaaggctgcacttaycaaaaatattatctgctcagcagca

SEQ ID NO: 389 - DNA, 51 bp, Homo sapiens
tgataccgacatacactcaactgatyagattttaatttagcaagcatcatt

SEQ ID NO: 390 - DNA, 51 bp, Homo sapiens
tttctaagcctgctttatttctaacycaacttttcactacaaggagaggga

SEQ ID NO: 391 - DNA, 51 bp, Homo sapiens
tcaaataactttccaagactccacaytatccaacttagtgaaccttcatca

SEQ ID NO: 392 - DNA, 51 bp, Homo sapiens
ataattaactttctagtttgtgttayacttctctttgcttaaaaaataaat

SEQ ID NO: 393 - DNA, 51 bp, Homo sapiens
aactaaaggtggatatgccatgtacragagagtcttcaaaaagttcataga

SEQ ID NO: 394 - DNA, 51 bp, Homo sapiens
acattaatgtcatacaggactacaamaaattagggttcagagttatagata

SEQ ID NO: 395 - DNA, 51 bp, Homo sapiens
tatgatgaatttatagatgcaaagaygattgacaaaagcctggcctttaaa

SEQ ID NO: 396 - DNA, 51 bp, Homo sapiens
gctaaaatgttggactatcataaaaycattttcaaagtgtgagacaagaag

SEQ ID NO: 397 - DNA, 51 bp, Homo sapiens
gtaattatcgacgtgaaacgcaaacrcactcttcttttggaatgttctaat

SEQ ID NO: 398 - DNA, 51 bp, Homo sapiens
aaatcatatcaacctaaaagactgaygagtgttaatttaagtaattttgat

SEQ ID NO: 399 - DNA, 51 bp, Homo sapiens
caatttctccaagttttccaaaacartaagtagctaatgtaatcttagcaa

SEQ ID NO: 400 - DNA, 51 bp, Homo sapiens
ttaaattaccaaccaacatctaagargtccaaactcctctctcccttcaaa

SEQ ID NO: 401 - DNA, 51 bp, Homo sapiens
aaaactcactctccagaatgatgaartaataaagaatggtatttgaagcca

SEQ ID NO: 402 - DNA, 51 bp, Homo sapiens
cctataccgtgaagctggccaaaacrgtataatctcaatatttacacttaa

SEQ ID NO: 403 - DNA, 51 bp, Homo sapiens
gtattcaattgctatggtaattttgkagcgccaaagacaaattttctaaaa

SEQ ID NO: 404 - DNA, 51 bp, Homo sapiens
ttttaatgagatgtggcttaataggwaaattttacttgttactagatgtca

SEQ ID NO: 405 - DNA, 51 bp, Homo sapiens
gtctgttgcaaaatggcctagggaargaagtcagtagaaggcaggccactt

SEQ ID NO: 406 - DNA, 51 bp, Homo sapiens
ccacttcctctatcatccatctatckctatgtaccggcctatctatatata

SEQ ID NO: 407 - DNA, 51 bp, Homo sapiens
acaggaaagatttcaggaaataataygagtcacacatgttggttctatagc

SEQ ID NO: 408 - DNA, 51 bp, Homo sapiens
cttttataatttgtttaaagttaacrctgaatatgacatatttttaaataa

SEQ ID NO: 409 - DNA, 51 bp, Homo sapiens
ttattcagtcctattttcattcatargtcagccagtcactgtatctcctat

SEQ ID NO: 410 - DNA, 51 bp, Homo sapiens
ttaatatcattttcttttggtggaastacacaatgatttaaaaattcaatt

SEQ ID NO: 411 - DNA, 51 bp, Homo sapiens
tacctcaacaaatcggaagccaaacrggatacctttaccaaagctaccgcc

SEQ ID NO: 412 - DNA, 51 bp, Homo sapiens
agagtgtcagttatgtatcttaataycatatcatactctacatagtcacag

SEQ ID NO: 413 - DNA, 51 bp, Homo sapiens
gaatttcaggtttctcggtcctaaarattgagacataggaaaagagaacac

SEQ ID NO: 414 - DNA, 51 bp, Homo sapiens
aatcatacatagcttagggatatagraaaacactgtcccaactctcagaag

SEQ ID NO: 415 - DNA, 51 bp, Homo sapiens
tggagctgacgcttcctcaatcatasggtgaaccgtcccagggtagctggg

SEQ ID NO: 416 - DNA, 51 bp, Homo sapiens
gacctcagtacagcattccaggccamagatccagaatgttcccttactggg

SEQ ID NO: 417 - DNA, 51 bp, Homo sapiens
gacacttctaatgtttcatacagcastcaagttcctagtcctcttttaatt

SEQ ID NO: 418 - DNA, 51 bp, Homo sapiens
tccccaacgatgatggcccttcttarcccaagcctcactttatggtttctt

SEQ ID NO: 419 - DNA, 51 bp, Homo sapiens
aacaattctgcctttttctctgtatraattgaaaccatatataatttttaa

SEQ ID NO: 420 - DNA, 51 bp, Homo sapiens
aacctcatttgtaccaaattgcagaygatgtgtatacaaccccatcgtttg

SEQ ID NO: 421 - DNA, 51 bp, Homo sapiens
tactctatcctataattaaatgccastactgtttctccacataaccatccc

SEQ ID NO: 422 - DNA, 51 bp, Homo sapiens
actgatacatttcaagagataagggycacaaggagattttcactcaagttg

SEQ ID NO: 423 - DNA, 51 bp, Homo sapiens
ttgatttttcccaaagccatcttgtyaagtagtggaagtcatggacaatgg

SEQ ID NO: 424 - DNA, 51 bp, Homo sapiens
caatccattgtccatgacttccaatmaataatttgtcttaaaataatgttt

SEQ ID NO: 425 - DNA, 51 bp, Homo sapiens
ttcttaagggctgaaagcacctgtcratatattttccagagttccagttta

SEQ ID NO: 426 - DNA, 51 bp, Homo sapiens
ccaaagagacgaaatagatcttagasaccttagaatattaatatccttagg

SEQ ID NO: 427 - DNA, 51 bp, Homo sapiens
gagcattttgcaataattatcactaycatcattatttcaacttcatgaaat

SEQ ID NO: 428 - DNA, 51 bp, Homo sapiens
aaaaatctctaaaagcaatgacttaraaaatgacaagactctagatatcat

SEQ ID NO: 429 - DNA, 51 bp, Homo sapiens
cagtagcccattcaaggtatacaaarttaaaggaatttgtagagggttttg

SEQ ID NO: 430 - DNA, 51 bp, Homo sapiens
tctcatgttgtatgagcctgtgtcaygttagatgaaaaatgcatacatata

SEQ ID NO: 431 - DNA, 51 bp, Homo sapiens
ggattaatatcaattgaaagctagtraaggtttaccccatgttagtcacct

SEQ ID NO: 432 - DNA, 51 bp, Homo sapiens
ttgaattatttatctttagtaaatcraaatgtgttttaagtgtgctcaccc

SEQ ID NO: 433 - DNA, 51 bp, Homo sapiens
ataaataattcaagccttcaataaamaggactgtgagttttttgaaagcaa

SEQ ID NO: 434 - DNA, 51 bp, Homo sapiens
catgaaaatgttagattaaatatgaracctgaatctagaagaaacaagctt

SEQ ID NO: 435 - DNA, 51 bp, Homo sapiens
gtcattattaactagcacttattgastattgattatgtgccaggaatcatg

SEQ ID NO: 436 - DNA, 51 bp, Homo sapiens
gcttttgaagcataagtgattacaayttggcaatggtgtcaggcagaactg

SEQ ID NO: 437 - DNA, 51 bp, Homo sapiens
ccactgtctttcaccagagatgaaaktaacaatgtactcaatgtcatctag

SEQ ID NO: 438 - DNA, 51 bp, Homo sapiens
gcgcttagtagccttagaagcatcaygtatttcgaaagtggtattttaccc

SEQ ID NO: 439 - DNA, 51 bp, Homo sapiens
aaattaagatacgtctagttatacasattgacccagaacccaggcatgtgg

SEQ ID NO: 440 - DNA, 51 bp, Homo sapiens
tctctcttagggaaattgttctttcratgtgtccagagacccgtttatttt

SEQ ID NO: 441 - DNA, 51 bp, Homo sapiens
actccaattgttccccctttttatckctattggtcatgtctggacttggaa

SEQ ID NO: 442 - DNA, 51 bp, Homo sapiens
ctgccctcaactcatggtttctgcaytgaaaaacgtgaaattgagattgtc

SEQ ID NO: 443 - DNA, 51 bp, Homo sapiens
tcctctcctagggtcatcaaggacckatatgatttgtgcccatttgtatct

SEQ ID NO: 444 - DNA, 51 bp, Homo sapiens
aacgatcagagcagaaataaatgaaractaacaacaataaaagcatcaaaa

SEQ ID NO: 445 - DNA, 51 bp, Homo sapiens
ctgccaaagcaatatcttctaaatamtccacccaatgccccatgaattaca

SEQ ID NO: 446 - DNA, 51 bp, Homo sapiens
aaaaaatctgaaaagatcatgatcgkgtaaagagattgattccacaataaa

SEQ ID NO: 447 - DNA, 51 bp, Homo sapiens
agtgcccttataaaatagacccatargaactcgttcacccctcccaccaca

SEQ ID NO: 448 - DNA, 51 bp, Homo sapiens
agatactaaacaactacgtactataygaccaataccggaaaaatattccat

SEQ ID NO: 449 - DNA, 51 bp, Homo sapiens
aggaggagacaatggcttatgtgacytatgagacagcatagagcaaccaaa

SEQ ID NO: 450 - DNA, 51 bp, Homo sapiens
ctattgttcaactgttcactgaagayaggctttgcagttacatttcccctt

SEQ ID NO: 451 - DNA, 51 bp, Homo sapiens
atatttgcaaaaaagaaaaatccaaytaaatgattgtcataaggtttaaat

SEQ ID NO: 452 - DNA, 51 bp, Homo sapiens
tgttctcctcttccttttacatacamgactggactgcatctggctcttcca

SEQ ID NO: 453 - DNA, 51 bp, Homo sapiens
tgggggaggtcccagactccttaatwactagatctcacataaactcattac

SEQ ID NO: 454 - DNA, 51 bp, Homo sapiens
aaaaaaaaatagcacttgcatcacayagatactcctaggaaaatgcaaatt

SEQ ID NO: 455 - DNA, 51 bp, Homo sapiens
tcagttcttttcgaatgtatcatacygctctttctctataggagataagag

SEQ ID NO: 456 - DNA, 51 bp, Homo sapiens
cctctcttcttcccaatgtcatggaygatgaaaagaactggcctcatatgg

SEQ ID NO: 457 - DNA, 51 bp, Homo sapiens
aagcattaaccatgggacaccattckaaagatttttctaaataattgtata

SEQ ID NO: 458 - DNA, 51 bp, Homo sapiens
catggaaagctatgaaggcattagarcggatatataaatttctggtgatgt

SEQ ID NO: 459 - DNA, 51 bp, Homo sapiens
catactcactttgttcttccattcaycttgataatttgacaccatactaac

SEQ ID NO: 460 - DNA, 51 bp, Homo sapiens
aataataaagaaactgatgtcaagcratttatctgattcagattacagtct

SEQ ID NO: 461 - DNA, 51 bp, Homo sapiens
gtatttctcttggctattttatccartcactttttggtggcatttacaatg

SEQ ID NO: 462 - DNA, 51 bp, Homo sapiens
aaccctcatcactcactttggaggakaccatgctaccatgtcacagtgtaa

SEQ ID NO: 463 - DNA, 51 bp, Homo sapiens
ttgaattctactcattgcaaggagaygctttaaagacagaggggcagcaag

SEQ ID NO: 464 - DNA, 51 bp, Homo sapiens
gccatgtacagcactatcaggactayggtgaggcgaccgtagatagctggg

SEQ ID NO: 465 - DNA, 51 bp, Homo sapiens
ttacagttaaataaattatgttaaaracaaaggtaatacatactctaaatg

SEQ ID NO: 466 - DNA, 51 bp, Homo sapiens
gaaggggtatctgtgaaggtattgcyaaggcaccaggcagtgaggaagatg

SEQ ID NO: 467 - DNA, 51 bp, Homo sapiens
taagtgggtgacttgagagattcaartgtatgatttaacctttgacttggt

SEQ ID NO: 468 - DNA, 51 bp, Homo sapiens
taagcaggcagaatacagaatcatcraattttaagataggccaattgaaaa

SEQ ID NO: 469 - DNA, 51 bp, Homo sapiens
tgtttaaacatgcaagaataagagcrgatgataatgaacatgtgaaagatg

SEQ ID NO: 470 - DNA, 51 bp, Homo sapiens
gaatccttccacgaaacttcaaataygtgggttaaattccggaaagcctct

SEQ ID NO: 471 - DNA, 51 bp, Homo sapiens
gatgcatagattgtgactgatttagraagtaccagaaagatcctctattca

SEQ ID NO: 472 - DNA, 51 bp, Homo sapiens
ctagagtctatcattaacacattackatacttgctttatgcatagctaccc

SEQ ID NO: 473 - DNA, 51 bp, Homo sapiens
gttggagagccaggcctgtatcccawctcaagtccaaattcagcactattt

SEQ ID NO: 474 - DNA, 51 bp, Homo sapiens
aaaaattttaattttaaaggagatasacaagaaatgagcatgtgtgaaagc

SEQ ID NO: 475 - DNA, 51 bp, Homo sapiens
catgccagtgacaattttttattcarcaggactctcttacacatatgctgt

SEQ ID NO: 476 - DNA, 51 bp, Homo sapiens
aacctttttatcattcagaaagttayatcattcagaaagcacccctccagt

SEQ ID NO: 477 - DNA, 51 bp, Homo sapiens
tttcttgattgtccattataattccrtatgttgtatcttctgctgtagtat

SEQ ID NO: 478 - DNA, 51 bp, Homo sapiens
gttgagaaatttgttctaacattacrgcagaaattttcaagaaggcacaat

SEQ ID NO: 479 - DNA, 51 bp, Homo sapiens
ggtcttagaagctgcttcagaattarggtctctcttatcttgtttttcccc

SEQ ID NO: 480 - DNA, 51 bp, Homo sapiens
aatatgacacagattactaatggtgsatcttaataaaaatatattttatct

SEQ ID NO: 481 - DNA, 51 bp, Homo sapiens
ggatgagcatgagccgaggcaaaaawtgggaagtcaagtggaggaatgata

SEQ ID NO: 482 - DNA, 51 bp, Homo sapiens
acataaaatcaatacatccatacaayttacagagaggtgctaggcactgtc

SEQ ID NO: 483 - DNA, 51 bp, Homo sapiens
caaagggagaaatgattgagaaatayacctgcttggaactagaggaacatc

SEQ ID NO: 484 - DNA, 51 bp, Homo sapiens
gctaaggctgactgagacaaatttamgtattagttggcagctgtaatcgtt

SEQ ID NO: 485 - DNA, 51 bp, Homo sapiens
ccatcacgtgactggcgctaaaatayaaacgttcccattttgtgtggtata

SEQ ID NO: 486 - DNA, 51 bp, Homo sapiens
catagtaaacacctaataaatattgmagactgaatgactataataggaaat

SEQ ID NO: 487 - DNA, 51 bp, Homo sapiens
gagtttacagcagggaattacaaatkataagggagatcactgctatatatc

SEQ ID NO: 488 - DNA, 51 bp, Homo sapiens
acttaaaatttccacatgcagattgsagtttacagcagggaattacaaatt

SEQ ID NO: 489 - DNA, 51 bp, Homo sapiens
caaagtctggacaagaaaccaacacsgtatttacatgatcctcaatttctg

SEQ ID NO: 490 - DNA, 51 bp, Homo sapiens
aaaatctcaagcccttgtatgaaacraagaagtgtggcctgtccctacgta

SEQ ID NO: 491 - DNA, 51 bp, Homo sapiens
ctgtcaaaggatgagctgaataatayggttgaaagagtcctgctgttattt

SEQ ID NO: 492 - DNA, 51 bp, Homo sapiens
gcaagccccaatgttctctgattcakctaaacttatttaaggtttttttaa

SEQ ID NO: 493 - DNA, 51 bp, Homo sapiens
aacattctgaaaaatgcactgaaaartaagtgtaggtactaagaatgacaa

SEQ ID NO: 494 - DNA, 51 bp, Homo sapiens
tccaacttgcaagcatttggaaagawccagtgattgattaaaaaaggttga

SEQ ID NO: 495 - DNA, 51 bp, Homo sapiens
ctgagaatctcttgctcctttaccasgctgtaggaatttggaaagacagag

SEQ ID NO: 496 - DNA, 51 bp, Homo sapiens
ttttggaaatgtgttaccagatactyaatttactgcaagttcattttggaa

SEQ ID NO: 497 - DNA, 51 bp, Homo sapiens
agatggaggctcacactgaagattaygccctgattcatgtaaaaatatttg

SEQ ID NO: 498 - DNA, 51 bp, Homo sapiens
ggactctttcttaaaatatggctagmatcctccctttgtccttgctcttca

SEQ ID NO: 499 - DNA, 51 bp, Homo sapiens
cacctcattctattcttaacactttygtcactaagcacattgccagtttac

SEQ ID NO: 500 - DNA, 51 bp, Homo sapiens
caaataattttttgcatgttgaatayagtacttctaatttctttctccaga

SEQ ID NO: 501 - DNA, 51 bp, Homo sapiens
agtgcaggggagaggagccacagaamgccgaacctcagaggggactcccag

SEQ ID NO: 502 - DNA, 51 bp, Homo sapiens
cagctgcagaggtgagctgccaaggytacatcattttgtgggtgggccaca

SEQ ID NO: 503 - DNA, 51 bp, Homo sapiens
agggttgtatcacccggctgacagayaaatataaagtaaaacctcagatgc

SEQ ID NO: 504 - DNA, 51 bp, Homo sapiens
tcgggtctattttatggtaatgtackatttgtcggagtgtcaaaatgactt

SEQ ID NO: 505 - DNA, 51 bp, Homo sapiens
atagatatctaaaggttatttgaagrccaatctggcatatagaatattctt

SEQ ID NO: 506 - DNA, 51 bp, Homo sapiens
caattacatggaggtaacttcatgaraagaataagatcagtcatactcaag

SEQ ID NO: 507 - DNA, 51 bp, Homo sapiens
tggtaagaacagattaaaccttagartgtgaacgcctaatagattcatctt

SEQ ID NO: 508 - DNA, 51 bp, Homo sapiens
taaatttatgacctttatgtcaggaytatatttgatttcagtatgaaaatg

SEQ ID NO: 509 - DNA, 51 bp, Homo sapiens
cctctcaagagagtcaactcttctcwctgtaatagctctatagttctagtt

SEQ ID NO: 510 - DNA, 51 bp, Homo sapiens
gttcagggctggttaagccagaggcycagtgaagttatcgcaaggtgctgt

SEQ ID NO: 511 - DNA, 51 bp, Homo sapiens
tgaaatgcatttatctttggtgcaarttattttattgtctcttctcatacc

SEQ ID NO: 512 - DNA, 51 bp, Homo sapiens
gtgcaagttattttattgtctcttcycatacctcttatccatttttttgcc

SEQ ID NO: 513 - DNA, 51 bp, Homo sapiens
aagaattgcccgtctttgatgccagragtaaccctagccgtgaaattcctt

SEQ ID NO: 514 - DNA, 51 bp, Homo sapiens
aattcttagagactagtggttccaaragtgaaatcccaagaccagtagaag

SEQ ID NO: 515 - DNA, 51 bp, Homo sapiens
aagtcattactgaacatcacatttaraggagatatatatattatatagccc

SEQ ID NO: 516 - DNA, 51 bp, Homo sapiens
attccaaatataaaactatgttacaytgaaagacacccctcccaagagatt

SEQ ID NO: 517 - DNA, 51 bp, Homo sapiens
tttcaaaatcctcagtgggcaaaaasctaccaacacagccacaattttttt

SEQ ID NO: 518 - DNA, 51 bp, Homo sapiens
agtattgaggatttaaaggaagaaawtattccaagtaaaagtaactgtata

SEQ ID NO: 519 - DNA, 51 bp, Homo sapiens
tcttgacagttgtttctttaacaatratgtataatccaataatgtataatc

SEQ ID NO: 520 - DNA, 51 bp, Homo sapiens
caatctacaaccctttctatggcaaycttgacagctcagtcattttcctca

SEQ ID NO: 521 - DNA, 51 bp, Homo sapiens
tgaatgtcggggggcactctgaagaygtaattgtatcattgtgtcctgctc

SEQ ID NO: 522 - DNA, 51 bp, Homo sapiens
taatctcctctcatgtggtaagttaygatagaattatttaaaaaattgctg

SEQ ID NO: 523 - DNA, 51 bp, Homo sapiens
gaaatatataatccatgtgaagagayagccgtaaggaaaggtaatacattt

SEQ ID NO: 524 - DNA, 51 bp, Homo sapiens
aacaactcgtctacttcctgtttcaratgctccgatattctcatatagcta

SEQ ID NO: 525 - DNA, 51 bp, Homo sapiens
caaatttaaaaggggcaaggaaagasaatcccaacatgtacttaaaggaag

SEQ ID NO: 526 - DNA, 51 bp, Homo sapiens
gataaatattttgcattacttttaaycctagacattatagagatactcagt

SEQ ID NO: 527 - DNA, 51 bp, Homo sapiens
atcttctgttattcacaaatacataygaaagagccagaatacgcccagtga

SEQ ID NO: 528 - DNA, 51 bp, Homo sapiens
atatgtatttgtgaataacagaagayacaatgacatggtgatggtaacaca

SEQ ID NO: 529 - DNA, 51 bp, Homo sapiens
atctgacatttccctacctgtataaytaatctagtgagtggtctacttagc

SEQ ID NO: 530 - DNA, 51 bp, Homo sapiens
ctttttttttctttcctgcagtaaamcttcctgcgttagcagtgattgatc

SEQ ID NO: 531 - DNA, 51 bp, Homo sapiens
gctaagatgtctaaaaagctatattyctacatttcatttctgactcttttc

SEQ ID NO: 532 - DNA, 51 bp, Homo sapiens
gatgacagagttgattcttcttctayggttccactaatttccaaatttctt

SEQ ID NO: 533 - DNA, 51 bp, Homo sapiens
gagtggcctggccatggcattgtgtrtagttgctgatcatggtcttacaca

SEQ ID NO: 534 - DNA, 51 bp, Homo sapiens
gttagtaagattagtaagaaaataaygacaaattcgcctttagaaatcttt

SEQ ID NO: 535 - DNA, 51 bp, Homo sapiens
aaagccaaagaaaagtagagtgagaytatgttgcaggttaggatacatatc

SEQ ID NO: 536 - DNA, 51 bp, Homo sapiens
tcatcagaattaacccattgacccawatggatctttgacacttcctaagtc

SEQ ID NO: 537 - DNA, 51 bp, Homo sapiens
tttaccagaagcccagccctggcaayagcagtaacagcagcaatgacaata

SEQ ID NO: 538 - DNA, 51 bp, Homo sapiens
ctatacagcatgggcatgccagacawagagaagattcatttaccagcatgg

SEQ ID NO: 539 - DNA, 51 bp, Homo sapiens
aatagattattcagctacaccttctwacatatcctgcaaccagatgtagtc

SEQ ID NO: 540 - DNA, 51 bp, Homo sapiens
attaaaacaataagctaaatttcacrgactgttaacctctgaacaactgct

SEQ ID NO: 541 - DNA, 51 bp, Homo sapiens
ctttattttcattcatttgcacatayccttattgaaaagtataaacaaagt

SEQ ID NO: 542 - DNA, 51 bp, Homo sapiens
acagaaagaaatattttatggccaayataatagtgtatcaaaccactttta

SEQ ID NO: 543 - DNA, 51 bp, Homo sapiens
gcagggcccctgcctcactttgggasaagaaagccttagtggggagaaata

SEQ ID NO: 544 - DNA, 51 bp, Homo sapiens
attattcttagaaagtacattggggyagatcaatcaggataggatttgtct

SEQ ID NO: 545 - DNA, 51 bp, Homo sapiens
attcctgactcctgtgtccatgaaasacatcactaatcaatctggtactct

SEQ ID NO: 546 - DNA, 51 bp, Homo sapiens
tattatgtgtcttggcttaaagggakaaacatcaatattaacaccactctt

SEQ ID NO: 547 - DNA, 51 bp, Homo sapiens
cagttaatgggctggttactgtcacrcacaagcactgtcccagcatttgcc

SEQ ID NO: 548 - DNA, 51 bp, Homo sapiens
ataatgctgtgactttgtattagccrggagtcctgaattatcaaaagattt

SEQ ID NO: 549 - DNA, 51 bp, Homo sapiens
ggaaattaattaaatagaggcaacarattgggatagtaaacctatttgtaa

SEQ ID NO: 550 - DNA, 51 bp, Homo sapiens
aaaaaaatctcaacatgaataaaaakcagaatagaaaattatatatgcacc

SEQ ID NO: 551 - DNA, 51 bp, Homo sapiens
caccctaccactaacttgcttcttayctgaaaagattttggagaagagctt

SEQ ID NO: 552 - DNA, 51 bp, Homo sapiens
ttatgagtccccgactaaagcctaaragaatcaactccaaagaaacatagc

SEQ ID NO: 553 - DNA, 51 bp, Homo sapiens
caagaaggagaagttgaactgagtargtgaagaaaaatcaagggccagaag

SEQ ID NO: 554 - DNA, 51 bp, Homo sapiens
ccctaacacatagtcatacaggtaartaatatctctcaatctctatctgta

SEQ ID NO: 555 - DNA, 51 bp, Homo sapiens
ctagggaaaaaccattatggctgtaygctctattctgtctctcatcctgac

SEQ ID NO: 556 - DNA, 51 bp, Homo sapiens
tgtgctcagggttatgcaattaaacyacattctatatttaaaattctccct

SEQ ID NO: 557 - DNA, 51 bp, Homo sapiens
aacaatcagatctctcaggaacgaayagagaaagagttcatttattactgc

SEQ ID NO: 558 - DNA, 51 bp, Homo sapiens
cacctctctatccctaatatctggaraattacctagtatatagtaaatgct

SEQ ID NO: 559 - DNA, 51 bp, Homo sapiens
ttaatgtagaacctgatgacactgartaggaccatgatttatttctaagtt

SEQ ID NO: 560 - DNA, 51 bp, Homo sapiens
agttcattttagataatatttgaaayggaagaagtagctgaggacagaaga

SEQ ID NO: 561 - DNA, 51 bp, Homo sapiens
aaaaatgttaatccttatgagggtaygatgaagtagacacgcagtaactat

SEQ ID NO: 562 - DNA, 51 bp, Homo sapiens
actttaagaattagtgctttaacacratttttactggctatctaaaaaatt

SEQ ID NO: 563 - DNA, 51 bp, Homo sapiens
caagacaccatgacatacacattaayagaacagagtagagagtccagaaaa

SEQ ID NO: 564 - DNA, 51 bp, Homo sapiens
atttcttgctagtattgctgaaatasacctctaataggttcctaaccttac

SEQ ID NO: 565 - DNA, 51 bp, Homo sapiens
ttatgacgtcattataagaaagaccrtatgcaagctaaaactataagatac

SEQ ID NO: 566 - DNA, 51 bp, Homo sapiens
cttgaacttttttgatcagatgtgayattctccatttttactggcattatc

SEQ ID NO: 567 - DNA, 51 bp, Homo sapiens
ggaggtgtggtgtcagaggttaatcraagacagaagttgggtaggctaaga

SEQ ID NO: 568 - DNA, 51 bp, Homo sapiens
aagaaagtgaaaagacaaccaatccrctgaataagagagactatttacaaa

SEQ ID NO: 569 - DNA, 51 bp, Homo sapiens
aataagctctatatttttgaaatacsaagactaagatgtaaacttcgatct

SEQ ID NO: 570 - DNA, 51 bp, Homo sapiens
cataaggcatgattgtaccaaattawgtgctaagaacctcaaatacacttc

SEQ ID NO: 571 - DNA, 51 bp, Homo sapiens
tatagctgcactgaaaattcaatagragcactcaacaacatactagtttaa

SEQ ID NO: 572 - DNA, 51 bp, Homo sapiens
ctatacaaattgcatttatcatcaasgcacaaggcccagctcccccaaagc

SEQ ID NO: 573 - DNA, 51 bp, Homo sapiens
tttaagagcatttgcttggccataayaatacaactaacagtgatcgaaaac

SEQ ID NO: 574 - DNA, 51 bp, Homo sapiens
gttcttcccagtgatttcagggaagragactcggcttatagctcaatgttg

SEQ ID NO: 575 - DNA, 51 bp, Homo sapiens
ctgtttattttatgctgtagccatayctttcaaaactcctcatccttccca

SEQ ID NO: 576 - DNA, 51 bp, Homo sapiens
cgcaatagagattccaagatgtaggmcaagggaccactttatttttctctg

SEQ ID NO: 577 - DNA, 51 bp, Homo sapiens
tattcagtctttaacactccagatgmacagggtactaagatgactatgtct

SEQ ID NO: 578 - DNA, 51 bp, Homo sapiens
tagaaaacttcaaatgacatctccayacctctgagtaatatgaatctcctg

SEQ ID NO: 579 - DNA, 51 bp, Homo sapiens
tcagaggtatggagatgtcatttgargttttctatagaggtctcactaggg

SEQ ID NO: 580 - DNA, 51 bp, Homo sapiens
tgctacaacttgtgaagctggcaagrattgaaccatattgcctaagatcct

SEQ ID NO: 581 - DNA, 51 bp, Homo sapiens
gccacaaaccatctcagtcataccamattctcgtcccagtctgtatcctta

SEQ ID NO: 582 - DNA, 51 bp, Homo sapiens
ctgcaggcctagtaacttgcttaccyagtcggatacagcagaaatgatgtt

SEQ ID NO: 583 - DNA, 51 bp, Homo sapiens
atcttggcagacatagacagtgttgraatgtagataattagatttatactt

SEQ ID NO: 584 - DNA, 51 bp, Homo sapiens
cctgagacatccaggctatgattgayaattaccatgtaagagctcctaagg

SEQ ID NO: 585 - DNA, 51 bp, Homo sapiens
tccatctcaggtcaaaatccaatgayctagtacatactctgaatcttttca

SEQ ID NO: 586 - DNA, 51 bp, Homo sapiens
aaagtcttcaatacatgactaataayggcataaaagaaccaataaaccatt

SEQ ID NO: 587 - DNA, 51 bp, Homo sapiens
tattccatgtgtagtatactatgaasatataaaaacacattttaaattttg

SEQ ID NO: 588 - DNA, 51 bp, Homo sapiens
tcctccttcatgaggttcatagtccratgaggtgacaaatattatttactc

SEQ ID NO: 589 - DNA, 51 bp, Homo sapiens
gtaaattttaggtgcttcttagcacsagaaaaagtaagtaactatatgaga

SEQ ID NO: 590 - DNA, 51 bp, Homo sapiens
acctaagccagggaattgactttccygattctcagcgtcttctttcttaaa

SEQ ID NO: 591 - DNA, 51 bp, Homo sapiens
tatattccaatttcttgaattcttarccatcatataaagatgcaaatcata

SEQ ID NO: 592 - DNA, 51 bp, Homo sapiens
aaaacaacgaataatattaatgacaratatcaaggccatactatcacagag

SEQ ID NO: 593 - DNA, 33 bp, Homo sapiens
gttttcaatttgctaartggatactaaaaatta

SEQ ID NO: 594 - DNA, 51 bp, Homo sapiens
aaaaacatctctttcctcaggcatasaaatttgcaaggtacaaatgtcttt

SEQ ID NO: 595 - DNA, 51 bp, Homo sapiens
cattcaaggaaaggaaaatgctgcaraaaactagtggaggtgtgaaggctt

SEQ ID NO: 596 - DNA, 51 bp, Homo sapiens
taacaagaactgtctggttcctagtstgctagttgatatctggtatcattc

SEQ ID NO: 597 - DNA, 51 bp, Homo sapiens
ttcccttactagactgaaaggtgcayattttattattttgttcccaatcaa

SEQ ID NO: 598 - DNA, 51 bp, Homo sapiens
gggacttactgtcatttcttatctayggatattaattagtaacgtgagaac

SEQ ID NO: 599 - DNA, 51 bp, Homo sapiens
gagaataccagtccttcaggattaargccccattcttataactttatttaa

SEQ ID NO: 600 - DNA, 51 bp, Homo sapiens
cttatgtttaaatatttaaactacargaataaatctcacttgatcaagggg

SEQ ID NO: 601 - DNA, 51 bp, Homo sapiens
agtctagcaattagagaattcaaaastcagaaaagcaaatttaacaactta

SEQ ID NO: 602 - DNA, 51 bp, Homo sapiens
gcctccaaaactgtgtaagaataaaygtctgttggtataagccacccagtt

SEQ ID NO: 603 - DNA, 51 bp, Homo sapiens
gatctgtcaatatcacaatccagcartatcagatgctgtttggctctgatt

SEQ ID NO: 604 - DNA, 51 bp, Homo sapiens
ttggctagatctcatcatagggccasatccaatgcaagggaggctgcaaaa

SEQ ID NO: 605 - DNA, 51 bp, Homo sapiens
ttttctccccttttagaactaccaasgtaatggaagctgaaagaggaggct

SEQ ID NO: 606 - DNA, 51 bp, Homo sapiens
gtatcttcttcgcaggaccatgtcayacgaccaaccctaactgcaaaaggt

SEQ ID NO: 607 - DNA, 51 bp, Homo sapiens
gttcaaagattacagagaaaattccracagcatctgtttcaacagggaggg

SEQ ID NO: 608 - DNA, 51 bp, Homo sapiens
gatccactatgcctggccttctttcraggttctggttgcaatttaggatta

SEQ ID NO: 609 - DNA, 51 bp, Homo sapiens
tccatccttcaccatagttaatatcrattagagctttgtggagttgtttta

SEQ ID NO: 610 - DNA, 51 bp, Homo sapiens
gcaacgctgttttgtaagagaatcgyattctgagtgagaaaatcaagatat

SEQ ID NO: 611 - DNA, 51 bp, Homo sapiens
ctgagagtcccaacactaaattcacyattaggtacctttggggctcagctg

SEQ ID NO: 612 - DNA, 51 bp, Homo sapiens
ccccgtcttgggtattctcttataamaatgcaagaacagcctaataaccct

SEQ ID NO: 613 - DNA, 51 bp, Homo sapiens
cataatggaaacaaatgttacatctsaaagctaaatgaaaaaaatgtataa

SEQ ID NO: 614 - DNA, 51 bp, Homo sapiens
ttctagtgagctgtgctaacccctayagtcttatggtctggatcctattga

SEQ ID NO: 615 - DNA, 51 bp, Homo sapiens
gtaaccaagcaaaccaaactcgacartgaaaagagatgcctctgtgatgtg

SEQ ID NO: 616 - DNA, 51 bp, Homo sapiens
aagagacttgtaaggtatattaatcraagggtgctgatggttagagtagat

SEQ ID NO: 617 - DNA, 51 bp, Homo sapiens
ctcctagagcatctccctctgatcartgaagaaaataattccaaaaagagt

SEQ ID NO: 618 - DNA, 51 bp, Homo sapiens
gtgtttattcttcttgtcttagtgaygatttttcttgtgattgtcccatcg

SEQ ID NO: 619 - DNA, 51 bp, Homo sapiens
atgagacaatggtatagttcacataygaatttggagtacttttcaggtttt

SEQ ID NO: 620 - DNA, 51 bp, Homo sapiens
tttttaaaaatatatttggacttaamatattatgatatctagttaaataat

SEQ ID NO: 621 - DNA, 51 bp, Homo sapiens
gtggaaagggaaccgtaagtattcaygttctcctgtgttcctggccctgtg

SEQ ID NO: 622 - DNA, 51 bp, Homo sapiens
ccttgggttgtatattcctatccttytgtggacagtgaaacccctgccatc

SEQ ID NO: 623 - DNA, 51 bp, Homo sapiens
tttactagttaataatcagtaagcaracacttcagtgaatctttccagcta

SEQ ID NO: 624 - DNA, 51 bp, Homo sapiens
atatcaaaatccaaatagatgaagastcgccttacactttaccacactatt

SEQ ID NO: 625 - DNA, 51 bp, Homo sapiens
gcctgtaaatttacacaagctgtgcyagataggaacacagaacattgctgt

SEQ ID NO: 626 - DNA, 51 bp, Homo sapiens
aaaccaaagcacactacacaaccaaygctatagatacttttacaggaaaaa

SEQ ID NO: 627 - DNA, 51 bp, Homo sapiens
aaggaatataaaccatgctgttatamaggaatataagtcattctgtcataa

SEQ ID NO: 628 - DNA, 51 bp, Homo sapiens
ctcagacaagcaaacactgagagaartcatttcaattagaccagccttaca

SEQ ID NO: 629 - DNA, 51 bp, Homo sapiens
ccaatttctgcaaaaagctagcggarattctgaaggggattgcattgctgt

SEQ ID NO: 630 - DNA, 51 bp, Homo sapiens
gactcacagttccctagagtagcctyatccacagggtgttgtctatttgac

SEQ ID NO: 631 - DNA, 51 bp, Homo sapiens
tgctaatgtagaggatatatgctgcraggtgtccagaagatctagctaaga

SEQ ID NO: 632 - DNA, 51 bp, Homo sapiens
gtgaagaactaataaaatttctctcytaaacgctggtataattcactagtg

SEQ ID NO: 633 - DNA, 51 bp, Homo sapiens
accacagtatgtaagtccaattcaaygattttctaagtatgtaaaaaatga

SEQ ID NO: 634 - DNA, 51 bp, Homo sapiens
gttgctttcaacaaatgtcttctaaycaaagggctttttgtagtgggagag

SEQ ID NO: 635 - DNA, 51 bp, Homo sapiens
ctgctcctttgagtagctactagackgctgtttttcaccatgaatgcacat

SEQ ID NO: 636 - DNA, 51 bp, Homo sapiens
tgaagaataaaattgcaatatcaccrcttcctcctaatgaaggaaggattg

SEQ ID NO: 637 - DNA, 51 bp, Homo sapiens
tgcaaattcgtaacattctacctacwaacctctttctcagctctccctgcc

SEQ ID NO: 638 - DNA, 51 bp, Homo sapiens
tgttgcctaagtgtacaatgtttatraattctacagtacagtagtatacag

SEQ ID NO: 639 - DNA, 51 bp, Homo sapiens
actggatcttgtccaaaacaataaargtagtaacctctatgagtctgcaag

SEQ ID NO: 640 - DNA, 51 bp, Homo sapiens
tacattgcaatcacaatcaaaatcaragtgtcattttacagaaataggaaa

SEQ ID NO: 641 - DNA, 51 bp, Homo sapiens
taacttactttccctctttctatgarcaatggcaaataatctgtatggtta

SEQ ID NO: 642 - DNA, 51 bp, Homo sapiens
ggcatcattgctccaaaaatgatcartactaaataggttgaagacaagaca

SEQ ID NO: 643 - DNA, 51 bp, Homo sapiens
ataaagcaatcttcagggcattttgrtaaataaaggaaagcacaataactc

SEQ ID NO: 644 - DNA, 51 bp, Homo sapiens
tcaatccatgcacatgcttgccctasagaagtctcttatattaaagttcag

SEQ ID NO: 645 - DNA, 51 bp, Homo sapiens
aacgaaatgaacaaattttcggttayagatcagctctcaaacactaaagtc

SEQ ID NO: 646 - DNA, 51 bp, Homo sapiens
agactcatgtttttactagccacaakaaagtattaaactatttctatgaaa

SEQ ID NO: 647 - DNA, 51 bp, Homo sapiens
tttaaagcaaacccagatcttatacyggaatgtcttattagagtttttcct

SEQ ID NO: 648 - DNA, 51 bp, Homo sapiens
ttattaaatgcctgtaaggctgggasacttgtgttgactcctacacattta

SEQ ID NO: 649 - DNA, 51 bp, Homo sapiens
aaatggggtcacgagaataagtaaarggattagtctcatcagagtgaacgt

SEQ ID NO: 650 - DNA, 51 bp, Homo sapiens
aaaaaaaagcaaacaaaacaccccaytagaccagaagcttttgtattatgc

SEQ ID NO: 651 - DNA, 51 bp, Homo sapiens
atttattttcagaaataatttcaccyatgttactgtattttgggaatagcc

SEQ ID NO: 652 - DNA, 51 bp, Homo sapiens
ataacaggacctacattattttatawggtagacaaattgagtgtcagtagt

SEQ ID NO: 653 - DNA, 51 bp, Homo sapiens
gatttatttatttatcaaacccttayatagtacttgctatgtgtaatgata

SEQ ID NO: 654 - DNA, 51 bp, Homo sapiens
ggtttctcaagaatatgaagtgctcratgtaatttagagcaatctgcacag

SEQ ID NO: 655 - DNA, 51 bp, Homo sapiens
aaaaatcctctttgcaactccattgmattgtacataaacaagtcaagaaag

SEQ ID NO: 656 - DNA, 51 bp, Homo sapiens
atgttcatgattcaacaacctacacrgtacaatgtaagaattccacactgc

SEQ ID NO: 657 - DNA, 51 bp, Homo sapiens
atactaaaaagcaccacctcttctayaacgcagtactcttgctcaggccct

SEQ ID NO: 658 - DNA, 51 bp, Homo sapiens
ctctgtgctaaaaattgaggtcaccrctttcttcccattagtcctaatact

SEQ ID NO: 659 - DNA, 51 bp, Homo sapiens
ctagacatgctgtaaaacaattaaawaaggcattacagatacgattgtatt

SEQ ID NO: 660 - DNA, 51 bp, Homo sapiens
cctttgccgacataaaggcaccaaawcatccacaaatgttaatcatttctc

SEQ ID NO: 661 - DNA, 51 bp, Homo sapiens
taatcatttctcatgaccaatgtgaytaatatggtccaaatcacccttcag

SEQ ID NO: 662 - DNA, 51 bp, Homo sapiens
ccatatgagaacacagtctgactgayaccttgcttttagcctagtgagttc

SEQ ID NO: 663 - DNA, 51 bp, Homo sapiens
tcctttggtaactgcttgtaccaggyatcacggtaaacactgactggggat

SEQ ID NO: 664 - DNA, 51 bp, Homo sapiens
gtactgtgataagccctttgcatacracaacatccacattattatgttcat

SEQ ID NO: 665 - DNA, 51 bp, Homo sapiens
aatgttggagaggagcagtggaaacsgtcataaatcattggtgagtgtata

SEQ ID NO: 666 - DNA, 51 bp, Homo sapiens
gtgagggtaaggaagtcctcagtaaygtttcccttttaatgaaaagcagcc

SEQ ID NO: 667 - DNA, 51 bp, Homo sapiens
aaacccaaagtgctcaatagctataygttggatgacattgaactgaacact

SEQ ID NO: 668 - DNA, 51 bp, Homo sapiens
ttggatgacattgaactgaacactcrctgccatgctataaggcaaaaactg

SEQ ID NO: 669 - DNA, 51 bp, Homo sapiens
tcctaggacctcatatctgatatacraaagaaagtgatacagatttcccta

SEQ ID NO: 670 - DNA, 51 bp, Homo sapiens
ataaaaggtaatatatgtgtaaatgraatattattctacattacaaaggaa

SEQ ID NO: 671 - DNA, 51 bp, Homo sapiens
gagaacgaaagtgccagcgatttgaygatcggacactcttcgagcctgaag

SEQ ID NO: 672 - DNA, 51 bp, Homo sapiens
gcacgtgcagtaccaccacctgaagkcagggcatgaagccaaaaaggatta

SEQ ID NO: 673 - DNA, 51 bp, Homo sapiens
ataaaatgtcaagactgatattaaawctgggtggttgaaacaacagacatt

SEQ ID NO: 674 - DNA, 51 bp, Homo sapiens
attctaaatgcttactacccagttayaaatgctatgaatcttccaacatcc

SEQ ID NO: 675 - DNA, 51 bp, Homo sapiens
cagcactgtgctcaggactttgctckcattactattaccccttacataagc

SEQ ID NO: 676 - DNA, 51 bp, Homo sapiens
ggtaacattaaaaaatccataagtcrtttaggagcaaatctaactcatcta

SEQ ID NO: 677 - DNA, 51 bp, Homo sapiens
caagacctctcggcaggtctaataartactgtaattttgaaagagagatga

SEQ ID NO: 678 - DNA, 51 bp, Homo sapiens
ccccttctcaaacatattttctgaaygaatcactctcagcaggaaagtggt

SEQ ID NO: 679 - DNA, 51 bp, Homo sapiens
tgcttagacaactgcctagttttccrctgaagacacccagtaatccttctg

SEQ ID NO: 680 - DNA, 51 bp, Homo sapiens
cctctcaaggccaactataggctcckcttgccctgtacaaaactaagaaac

SEQ ID NO: 681 - DNA, 51 bp, Homo sapiens
ttcccaagccttatagcactaaacartggtttgcagttcaaggtgctcttg

SEQ ID NO: 682 - DNA, 51 bp, Homo sapiens
tattggcacaaactctgctagtatcraccatttccaatattagaaatagat

SEQ ID NO: 683 - DNA, 51 bp, Homo sapiens
tgatgaccaaagtaagaactgcccaratgccattctcacgttcctaagtca

SEQ ID NO: 684 - DNA, 51 bp, Homo sapiens
agatgctgagggccaaaagggtggakaatgtggatatcatgcatttgcctg

SEQ ID NO: 685 - DNA, 51 bp, Homo sapiens
ttagttgaggagtgaaccagatagcraaacttatgcccaagtctttaggct

SEQ ID NO: 686 - DNA, 51 bp, Homo sapiens
attactcacctcacattgtatgagcraaataaaattataccatgtaaaaaa

SEQ ID NO: 687 - DNA, 51 bp, Homo sapiens
taattgaaagagcaagaatttagagkcagaattatggtaaaagtcattctc

SEQ ID NO: 688 - DNA, 51 bp, Homo sapiens
ctctgtcactcttgcttttgttccawatcattaaaagaagtagcctcagag

SEQ ID NO: 689 - DNA, 51 bp, Homo sapiens
agggatgtctcctatttacactccartctgtgaaggatcctttgcttttct

SEQ ID NO: 690 - DNA, 51 bp, Homo sapiens
agtataattagaatacctggccttasattataagaaaggagaaatgacaag

SEQ ID NO: 691 - DNA, 51 bp, Homo sapiens
atactttaccttcctatctgacctayagtaaaacattttagaagcctttct

SEQ ID NO: 692 - DNA, 51 bp, Homo sapiens
catcacattattcattgtttcagatsagcatattctctttctagatctcat

SEQ ID NO: 693 - DNA, 51 bp, Homo sapiens
aaaatttaaaagggtacaagatcaayggtgtctcctaggatgttactgagc

SEQ ID NO: 694 - DNA, 51 bp, Homo sapiens
gtgactttattaacaaattgaatcaygtgcctcctcttctttagaatactt

SEQ ID NO: 695 - DNA, 51 bp, Homo sapiens
cctgtgttcatatattctaaatccarctagtcttctataaaaagttgaatg

SEQ ID NO: 696 - DNA, 51 bp, Homo sapiens
caatttaaagagaaagattataagcraatggtaatttctctaattgagtta

SEQ ID NO: 697 - DNA, 51 bp, Homo sapiens
cagtgaaagattgtgtgctttaggartaaaaatagctctttcaggtctcat

SEQ ID NO: 698 - DNA, 51 bp, Homo sapiens
ataatttctcataggtcaaagacaaygttattactgggtacatctagaata

SEQ ID NO: 699 - DNA, 51 bp, Homo sapiens
tagaatagactggcctcacggaagayattctccaacctaccagcctatcag

SEQ ID NO: 700 - DNA, 51 bp, Homo sapiens
acccactttatggctagatgggtccraaagcatgcagttcattataggtag

SEQ ID NO: 701 - DNA, 51 bp, Homo sapiens
gcttgacagtccatctagtgctatgragtatttcctaggtacacgatggaa

SEQ ID NO: 702 - DNA, 51 bp, Homo sapiens
tattgaaacagtatagatcttctcgycactgcagtaccctaataccattta

SEQ ID NO: 703 - DNA, 51 bp, Homo sapiens
aagcccaagctacaagaagagcccayatgtaggtttcagttgagcctagca

SEQ ID NO: 704 - DNA, 51 bp, Homo sapiens
cctttagcaaactcctaatgtgtcaktaaaaattctcaaggatgaaatagt

SEQ ID NO: 705 - DNA, 51 bp, Homo sapiens
aaatatgaggagaaatcattaacatyagaagcacatttaaggcttgatgtt

SEQ ID NO: 706 - DNA, 51 bp, Homo sapiens
aaccccctactttgtgtatatgaacmatgacaaggttacaggtgaatgtct

SEQ ID NO: 707 - DNA, 51 bp, Homo sapiens
taaggtcaagaaattttcccagagcraggagataaatattttaaaataatt

SEQ ID NO: 708 - DNA, 51 bp, Homo sapiens
ggtgacaagacttcatggtctaccartcagcccaaatgcccgccaaccttc

SEQ ID NO: 709 - DNA, 51 bp, Homo sapiens
acatttgaaatgtggctgtaggaaayggtgatggaaataatgggtacattt

SEQ ID NO: 710 - DNA, 51 bp, Homo sapiens
gacttatctgaaagttggagaataaygaaagagaagtaactggagaatgac

SEQ ID NO: 711 - DNA, 51 bp, Homo sapiens
aaatataacatgagaacaagactgcyatgactagaggtagctggcaggaac

SEQ ID NO: 712 - DNA, 51 bp, Homo sapiens
tatagatatgattctgaagacaagayacaatccaagcaatggctactgaga

SEQ ID NO: 713 - DNA, 51 bp, Homo sapiens
cctatattaaaccatgatttattacygaaattatactactaataattttct

SEQ ID NO: 714 - DNA, 51 bp, Homo sapiens
tatctgataagcagtgtatttgaacygtaccatggaaaacactgtattgat

SEQ ID NO: 715 - DNA, 51 bp, Homo sapiens
tgatgaggagaccatccacatggcaraggtatggtggccaagatggaagac

SEQ ID NO: 716 - DNA, 51 bp, Homo sapiens
atttgcataaattagtcatgggcccsccaattcaatgaaagaggaaaagag

SEQ ID NO: 717 - DNA, 51 bp, Homo sapiens
aaccctcaccacttgactgtattcartatcccttaatgataaaaactctca

SEQ ID NO: 718 - DNA, 51 bp, Homo sapiens
agtaagatcaattaacattctcttcyatattgaacataattagaatggcaa

SEQ ID NO: 719 - DNA, 51 bp, Homo sapiens
tatttaagcttttccctaattccagkactctaatattcagtactaattttt

SEQ ID NO: 720 - DNA, 51 bp, Homo sapiens
tgatcagatcagaatcagaacaatamtactcaggaaaatatcttgtatcac

SEQ ID NO: 721 - DNA, 51 bp, Homo sapiens
tttggtggttgttccaaagaaatackatcatccacaggcgtagattcagat

SEQ ID NO: 722 - DNA, 51 bp, Homo sapiens
tagaggtggggcctttgggaagcaaytagaattagataaggtcatcaaggt

SEQ ID NO: 723 - DNA, 51 bp, Homo sapiens
tcatttcatgcagcagcgaaattccyaaaaagtgttcttccagttttggtg

SEQ ID NO: 724 - DNA, 51 bp, Homo sapiens
aaccaagcccaattagacctcatcasttttccccaacattaacaaaccacg

SEQ ID NO: 725 - DNA, 51 bp, Homo sapiens
ttttcacaagaaaacagcacttaagscttctatatataatcactgttactt

SEQ ID NO: 726 - DNA, 51 bp, Homo sapiens
gtgtccaagctttttgctttaaagaycagcaagttgaaaccagagaattcc

SEQ ID NO: 727 - DNA, 51 bp, Homo sapiens
gaagtctgtaggaagctaagattcaraggtgaggaaggactgaggtttggg

SEQ ID NO: 728 - DNA, 51 bp, Homo sapiens
ctgattggaaatatcatccaaattgytttggctctgcccctccacattcaa

SEQ ID NO: 729 - DNA, 51 bp, Homo sapiens
gggcaatatgtgtttgataagtgaaygagaatgaatgaatgaagcttttcc

SEQ ID NO: 730 - DNA, 51 bp, Homo sapiens
atgtatgtacatgtatggatataaaycaatatgctaattctaacaatcaca

SEQ ID NO: 731 - DNA, 51 bp, Homo sapiens
aaaaagaaaggaatcaagcatacaasgatagaaaactcttaagttttatct

SEQ ID NO: 732 - DNA, 51 bp, Homo sapiens
tcctcctggctgcatatcggtgcackctgggtaactacagatgtcttcttt

SEQ ID NO: 733 - DNA, 51 bp, Homo sapiens
ttatattctcctataattttttaaaygagactagctctaggatcctgtggg

SEQ ID NO: 734 - DNA, 51 bp, Homo sapiens
gctattctcattcctgtgacttatarataatgctttatgttctttagtatt

SEQ ID NO: 735 - DNA, 51 bp, Homo sapiens
catttgcattcagggtctcacataawccaaaacttacacattgctttgcag

SEQ ID NO: 736 - DNA, 51 bp, Homo sapiens
tgcactttcagggtccttgatcatasgtcctggcctcttcttcagtcagag

SEQ ID NO: 737 - DNA, 51 bp, Homo sapiens
cagtttcctccccagagtgttaatartactaaaacaagttatttctgctgg

SEQ ID NO: 738 - DNA, 51 bp, Homo sapiens
caggtctaaagcaatgtcctggttaygaataagtggctggttcctcaaaaa

SEQ ID NO: 739 - DNA, 51 bp, Homo sapiens
ttgatttagagagagcttaccgatawaagtaagctgtctcactgtgtatcc

SEQ ID NO: 740 - DNA, 51 bp, Homo sapiens
gctgaaatgatattagatttagtgargtgagccgtatatcacaagtactcc

SEQ ID NO: 741 - DNA, 51 bp, Homo sapiens
agcaccgacaacattctttccttcawtcaaagtacataaatcacttactta

SEQ ID NO: 742 - DNA, 51 bp, Homo sapiens
attacttaaagacaagggtagtaagyattacaaattctgctggcaagagtt

SEQ ID NO: 743 - DNA, 51 bp, Homo sapiens
attaaacagttacctaatacaaagamaatctaggctgaatagcttcaaatg

SEQ ID NO: 744 - DNA, 51 bp, Homo sapiens
cacaatcctcatgaatgcgcttagarcccttacattagataccccagagaa

SEQ ID NO: 745 - DNA, 51 bp, Homo sapiens
aaggaatgtatcactgagtacattayctgagtatattcattagaagagtaa

SEQ ID NO: 746 - DNA, 51 bp, Homo sapiens
acatcaggcatgaagagatataatarctacactaccatagtgggtgaaggg

SEQ ID NO: 747 - DNA, 51 bp, Homo sapiens
tatgataagcttaactatctgaaccyggggtaaaaggctaataaccagcaa

SEQ ID NO: 748 - DNA, 51 bp, Homo sapiens
ggaggaagttcccgctaagcgtttayagtctgaaaggtacacactgtatga

SEQ ID NO: 749 - DNA, 51 bp, Homo sapiens
agaaaaaaaatttattacagcgtaargaattggcaaagtgattagggaggc

SEQ ID NO: 750 - DNA, 51 bp, Homo sapiens
acatctcattaaaaacatgtatagarcagtagaggtgatatgaaatagcca

SEQ ID NO: 751 - DNA, 51 bp, Homo sapiens
ttgtcatttttgtgatgcagagagartattttcaatagcatgtttaaagca

SEQ ID NO: 752 - DNA, 51 bp, Homo sapiens
agctactgggatgtgcagaactttayaatgtattgaaatccttggaagaga

SEQ ID NO: 753 - DNA, 51 bp, Homo sapiens
taagacacaagtccaatgaatggtaractgtagatgccttctttatttcct

SEQ ID NO: 754 - DNA, 51 bp, Homo sapiens
agtcatttctttataaaagtaaacaraagaagcatatcacagaatctctta

SEQ ID NO: 755 - DNA, 51 bp, Homo sapiens
ctttatcacctaaatgcgtgtccaartactatgttccacattccccaaaaa

SEQ ID NO: 756 - DNA, 51 bp, Homo sapiens
tgcctcctgtaaagtttttccggaartcaagccttctgtagtcagccagca

SEQ ID NO: 757 - DNA, 51 bp, Homo sapiens
gattacagcctaacactgcactaacmatggaaatcttatgcttaattcatc

SEQ ID NO: 758 - DNA, 51 bp, Homo sapiens
cctaattttttcctgcctttgataayggctctgtagcaactctagaaaaaa

SEQ ID NO: 759 - DNA, 51 bp, Homo sapiens
gtcattacatattttgagcaagtaartaagtaaaggtcttctgtgtgctct

SEQ ID NO: 760 - DNA, 51 bp, Homo sapiens
aaaagcattttggaaatagtggacamctaaggtttctcttaacgattacat

SEQ ID NO: 761 - DNA, 51 bp, Homo sapiens
ggctgaggtgggcggatcaccccaartcagaagttcgagaccagcctggcc

SEQ ID NO: 762 - DNA, 51 bp, Homo sapiens
agcttaaatgagtcactcccttgtawacatcctaaattctcttgctcctcc

SEQ ID NO: 763 - DNA, 51 bp, Homo sapiens
atagtttgccttttccagagtgccawacggttggaatcatacagtatgtag

SEQ ID NO: 764 - DNA, 51 bp, Homo sapiens
tctggaggctgggagttgaagataargataccagctgggtacgatccatgc

SEQ ID NO: 765 - DNA, 51 bp, Homo sapiens
atctagattgcgttgggtttcgtgaygacttcttaaatgaaacaccagagg

SEQ ID NO: 766 - DNA, 51 bp, Homo sapiens
acgtgattgagccccaactgatatayactaactccaaaacatcatgctaga

SEQ ID NO: 767 - DNA, 51 bp, Homo sapiens
actttctaaatatttgtaaatttagraggacacttataactaagccaagga

SEQ ID NO: 768 - DNA, 51 bp, Homo sapiens
cttagcttcccttcctttgggaaacsttccatcccttgctctagtctagag

SEQ ID NO: 769 - DNA, 51 bp, Homo sapiens
ccaatccccaacctgtaggccatgarcatcacgtcagtttttaaagccccc

SEQ ID NO: 770 - DNA, 51 bp, Homo sapiens
ctacaataaagagttttattccaaayaccagtgaaggactataaaaagggg

SEQ ID NO: 771 - DNA, 51 bp, Homo sapiens
gtaaaccagcatcaaatctttttacrgcctattgcagtttcaagaaacaca

SEQ ID NO: 772 - DNA, 51 bp, Homo sapiens
aaacaaacaaaaacgaaagggcttargataatggaataagatcagggtggc

SEQ ID NO: 773 - DNA, 51 bp, Homo sapiens
tatcagttctaagagttttctggtasagcctagatttttctataaaaccct

SEQ ID NO: 774 - DNA, 51 bp, Homo sapiens
agtttgcagcagggattcgatgagtraacacttggacggtgcctggaatag

SEQ ID NO: 775 - DNA, 51 bp, Homo sapiens
gtttatttgaagagttggtaggcccragactttttctgaaatgtttattga

SEQ ID NO: 776 - DNA, 51 bp, Homo sapiens
tccctttgcaataactcacaaactayacagttttcctccaaatgtgtgtaa

SEQ ID NO: 777 - DNA, 51 bp, Homo sapiens
tagcctggatgttttcaatctcagaygctagtcgtgttttttctctaaaat

SEQ ID NO: 778 - DNA, 51 bp, Homo sapiens
taagaagagaccagtaagaaagaacragatcaggacagtaggtcaaagagt

SEQ ID NO: 779 - DNA, 51 bp, Homo sapiens
atcaggcagctagtaattagcaacaytgggatccaaactcaggtctgtctg

SEQ ID NO: 780 - DNA, 51 bp, Homo sapiens
aaggaagcaagccaagttttaaacarcctgtctctaatgatctggtcagga

SEQ ID NO: 781 - DNA, 51 bp, Homo sapiens
atagttacaatactacgtaaaataarctaagataacagaggataggtgaat

SEQ ID NO: 782 - DNA, 51 bp, Homo sapiens
tcatcttccattcaactttcaaacaytgtccatactgttgtgtagtaacca

SEQ ID NO: 783 - DNA, 51 bp, Homo sapiens
aactgaggcagaagaaaattacatcrgatggaaacttggatctagacagat

SEQ ID NO: 784 - DNA, 51 bp, Homo sapiens
catatggtattcctaataaagagatwagataggaagaacgagaggtatttc

SEQ ID NO: 785 - DNA, 51 bp, Homo sapiens
ctatcacatcaccctatttcagtcayaaaacagtgctggtcatagagaagg

SEQ ID NO: 786 - DNA, 51 bp, Homo sapiens
attccctggcgttcctctcattcaasatgatcaaaatcatggaggcaggat

SEQ ID NO: 787 - DNA, 51 bp, Homo sapiens
aaacatgcattattggagttcttgcrtttaagcctgtttcacacatgtgcc

SEQ ID NO: 788 - DNA, 51 bp, Homo sapiens
caaaggcccagagggaggaacttgcrccttcataagagactcttggggctc

SEQ ID NO: 789 - DNA, 51 bp, Homo sapiens
ctggctcctccacttgccgtgtgtayaaccttggacggcagttcaagtggg

SEQ ID NO: 790 - DNA, 51 bp, Homo sapiens
agattccagattcagccttagcatawcagctgggagacttaacaatgagtc

SEQ ID NO: 791 - DNA, 51 bp, Homo sapiens
acagtattgaaacttcttattcgtgracatacgcaatcatggcatctgtga

SEQ ID NO: 792 - DNA, 51 bp, Homo sapiens
attattcttttgaactgaaagtcagkagaccttgattttagactcactcca

SEQ ID NO: 793 - DNA, 51 bp, Homo sapiens
agaatattatttaatggcatggaaasacaccacatgacaaaattcaattcc

SEQ ID NO: 794 - DNA, 51 bp, Homo sapiens
agaaagtggatggaaaagtaaaggcractatttcctgcacctcaggtatga

SEQ ID NO: 795 - DNA, 51 bp, Homo sapiens
tgtaatatttcagtacacaggtagartaggagacaggttttggtaattcat

SEQ ID NO: 796 - DNA, 51 bp, Homo sapiens
ctttagttcaaaattctgtgatatayaaataactcgaaataactgctaaca

SEQ ID NO: 797 - DNA, 51 bp, Homo sapiens
tatgccaaaagaggactgacagatayataagttttaagcttagggatgaga

SEQ ID NO: 798 - DNA, 51 bp, Homo sapiens
tgtttttatttctgaaagagtgggayactacctcaaagttgggaacatatt

SEQ ID NO: 799 - DNA, 51 bp, Homo sapiens
ttaatctatgcagatttactaaaggkaataagagtgaaactgatataacta

SEQ ID NO: 800 - DNA, 51 bp, Homo sapiens
cctctgcctcaccaaatcccaaagckagattccctcatggccctgcctgca

SEQ ID NO: 801 - DNA, 51 bp, Homo sapiens
tttactttggccctcctggcattcaygggagagtaagaagttatattgcat

SEQ ID NO: 802 - DNA, 51 bp, Homo sapiens
cctataaatattttttgtttaatccyaattcttcatattaggagacaaaga

SEQ ID NO: 803 - DNA, 51 bp, Homo sapiens
aggctaatttccaggaggcaaatgcyatttaactgtatatctttcagatct

SEQ ID NO: 804 - DNA, 51 bp, Homo sapiens
ttctctcttttctcaaagtgtttagwccatagtgagttactttgcttccaa

SEQ ID NO: 805 - DNA, 51 bp, Homo sapiens
catttaaatgctagataatatagtasgtgaatgagtaattttgaaagaaat

SEQ ID NO: 806 - DNA, 51 bp, Homo sapiens
tctggaattttattccgacctatgaygttaagacttcaaatctatggttga

SEQ ID NO: 807 - DNA, 51 bp, Homo sapiens
gggcttctaatgatcttgtagtgaaygtagtaacctatagatagttttgca

SEQ ID NO: 808 - DNA, 51 bp, Homo sapiens
gcaaggatttccaaactattaaaacyatcaagaaacagcagtgtaaggaac

SEQ ID NO: 809 - DNA, 51 bp, Homo sapiens
caagaaacattttacatacataacayagatgggcaatacaatttagtttaa

SEQ ID NO: 810 - DNA, 51 bp, Homo sapiens
acagtcataactgacaacattgaacracttcctatataaagagttagagtt

SEQ ID NO: 811 - DNA, 51 bp, Homo sapiens
aaggaaccatctcactgtgtgtaaayatgacttccaagctggccgtggctc

SEQ ID NO: 812 - DNA, 51 bp, Homo sapiens
ttaggactcggtgggaaataattgcsgaagatgaaacaaaggaggcagaaa

SEQ ID NO: 813 - DNA, 51 bp, Homo sapiens
atacaatattggatatatggatggastattcctcatttaatgactatcaaa

SEQ ID NO: 814 - DNA, 51 bp, Homo sapiens
cttcagaagcagagaatactaacagraattcaatttgagttgaacagtagg

SEQ ID NO: 815 - DNA, 51 bp, Homo sapiens
taaaccgtaaatgcaattgatgaaayattcaaatctacacaccgaaataaa

SEQ ID NO: 816 - DNA, 51 bp, Homo sapiens
aatcacaatcagaattccagtatacrctttttatcaaaattgacaacattc

SEQ ID NO: 817 - DNA, 51 bp, Homo sapiens
atccagtctgtggctcaatagaaacygtgaagaatcatcttctagaatagg

SEQ ID NO: 818 - DNA, 51 bp, Homo sapiens
tattctacatgtaaaggaattgattraagaagaaaaaaaagactcactatg

SEQ ID NO: 819 - DNA, 51 bp, Homo sapiens
ttataaaaaaacaaaaataacaacaygcaaaaaatatggagttgagtcatt

SEQ ID NO: 820 - DNA, 51 bp, Homo sapiens
cacctttccacccacctacaaataaytgtgtgtccaatccaatctatatat

SEQ ID NO: 821 - DNA, 51 bp, Homo sapiens
acaccactagtctgattctcatataygcacactgctgtaaaagactctcag

SEQ ID NO: 822 - DNA, 51 bp, Homo sapiens
tttgggaggagattcggctaaagtcyacgagggccacgtatttatatataa

SEQ ID NO: 823 - DNA, 51 bp, Homo sapiens
attgagaaatgaataaataaccgaasctcattcaggcctcaaaaggggtac

SEQ ID NO: 824 - DNA, 51 bp, Homo sapiens
tactttttcattatctttaccttaawctcttagcatatgatttatggactg

SEQ ID NO: 825 - DNA, 51 bp, Homo sapiens
atattttatagttctttccaagtcakaggtatagattactacaagttgaat

SEQ ID NO: 826 - DNA, 51 bp, Homo sapiens
cataatgattgcatggaaaaggtgaygctgaaggttgatagtaaccaaaat

SEQ ID NO: 827 - DNA, 51 bp, Homo sapiens
gtgtctcagacattcgtgcagcaaaygtggtaccaaaggactcacacaagg

SEQ ID NO: 828 - DNA, 51 bp, Homo sapiens
ggcacttgttaaaaagtaagtactaratcaatgtttgctgtctatctgtta

SEQ ID NO: 829 - DNA, 51 bp, Homo sapiens
tctttgcagtgagcgccacgtttccrgagtgtttggtccacaaagcgttat

SEQ ID NO: 830 - DNA, 51 bp, Homo sapiens
gtgacatctagaggccacattcccartaattaagagcaaatgccatagcta

SEQ ID NO: 831 - DNA, 51 bp, Homo sapiens
caagcttctgccctgatggttcctcrctttgccaggtctgtctgatccaag

SEQ ID NO: 832 - DNA, 51 bp, Homo sapiens
ttgactggatgttttggcattaagamattttgctaaattcaaaaagatgga

SEQ ID NO: 833 - DNA, 51 bp, Homo sapiens
gttaacttttatacaatgaataacayatctacttaatactcaaaatattgc

SEQ ID NO: 834 - DNA, 51 bp, Homo sapiens
agacagagggggacattttctgacasagcgttcaaggccagcagcagctca

SEQ ID NO: 835 - DNA, 51 bp, Homo sapiens
ctaggcggtccttgattaaattcatmactgatttgcttctagaaaactcct

SEQ ID NO: 836 - DNA, 51 bp, Homo sapiens
gcatgcaagaagcattgccaagtgasagaccagagagcaggggccatgtgg

SEQ ID NO: 837 - DNA, 51 bp, Homo sapiens
tttctctgagtgtgttgtatatcacragttagtaaattatggcccacggcc

SEQ ID NO: 838 - DNA, 51 bp, Homo sapiens
aaaccccaagttcagaggttaaaaartgggctcaaagtcatatagatagtc

SEQ ID NO: 839 - DNA, 51 bp, Homo sapiens
agagccaggattctcatagagtttgsagactagtgagaaagctagacatta

SEQ ID NO: 840 - DNA, 51 bp, Homo sapiens
tccatttaggatgaaaatgattgacyaactagaaatagatgagagctttct

SEQ ID NO: 841 - DNA, 51 bp, Homo sapiens
ttgaatcaatcttgcataccataagmcaaatcttacttggccatcatttat

SEQ ID NO: 842 - DNA, 51 bp, Homo sapiens
aagccaatacctcaacttgggtctamctgattacaaagtttatgttctttc

SEQ ID NO: 843 - DNA, 51 bp, Homo sapiens
tatattttgattgccaactaattcartaatactcttatttgagttggaaac

SEQ ID NO: 844 - DNA, 51 bp, Homo sapiens
tgccaataataaactgccaaactagraacctcaacttatttgttgtatagg

SEQ ID NO: 845 - DNA, 51 bp, Homo sapiens
ctaaaaatcacagctatttgagatgragtacaagtcaaaatgttgagtcta

SEQ ID NO: 846 - DNA, 51 bp, Homo sapiens
atgcattaaatagaatggaatttgaraagtagagtctagagtctatagatg

SEQ ID NO: 847 - DNA, 51 bp, Homo sapiens
ggtgctgccagcagttctctctgggwaggattgtggtataattctttccac

SEQ ID NO: 848 - DNA, 51 bp, Homo sapiens
tgtgcaaaaaaagtgatatatacaartactataaaatatatgcatgtacgt

SEQ ID NO: 849 - DNA, 51 bp, Homo sapiens
atgtctttcccatctcaattacaccractccatctgtttagtggttcaggc

SEQ ID NO: 850 - DNA, 51 bp, Homo sapiens
acatcagattttcagtgtagatcgamcagccttctgctggaagaagatgcc

SEQ ID NO: 851 - DNA, 51 bp, Homo sapiens
acaggaatttctagttatttcttcawgctctaccagtctttaagcttcata

SEQ ID NO: 852 - DNA, 51 bp, Homo sapiens
gcactccatcacagtgggagaggccrcccttggagatcctgtgaaccaagc

SEQ ID NO: 853 - DNA, 51 bp, Homo sapiens
acaatttcatacaattaaattaaccmgctcaaggtcactataattacagag

SEQ ID NO: 854 - DNA, 51 bp, Homo sapiens
ctgcctattctaggctaactataaartggtacttcatctttaaagcttttt

SEQ ID NO: 855 - DNA, 51 bp, Homo sapiens
gtgccactaacaaagaccactattayaggttagaagaagatgccatcctta

SEQ ID NO: 856 - DNA, 51 bp, Homo sapiens
aaaacctatttttgtaagccacataytaaaaatcactttaaaaaatcaaat

SEQ ID NO: 857 - DNA, 51 bp, Homo sapiens
gtcttgagaaattttccagaaataamcaggcaaatttctcaagagcaggca

SEQ ID NO: 858 - DNA, 51 bp, Homo sapiens
ccttcagtccatgatttttacattcrccctatgactatgtatccatgtcga

SEQ ID NO: 859 - DNA, 51 bp, Homo sapiens
tattgttaatgcaaacttaggtggtraaactatacagcaaatcaaggaaat

SEQ ID NO: 860 - DNA, 51 bp, Homo sapiens
gggccaggtaaggagcagtaggcctrgcaaggagagagaggagctggtgag

SEQ ID NO: 861 - DNA, 51 bp, Homo sapiens
atgagaagaatgtcaattctgttgtwcttacatggagtcttctctagatgt

SEQ ID NO: 862 - DNA, 51 bp, Homo sapiens
gactgcccatgtacctcattttctcrcttccttcctccttccacacctatt

SEQ ID NO: 863 - DNA, 51 bp, Homo sapiens
ggttaagtcaagatccccgaagttgwagaggtcaaatacaggttaaacatg

SEQ ID NO: 864 - DNA, 51 bp, Homo sapiens
atatttaatcatagaatacttctaawctccttctgacaaataaattgaact

SEQ ID NO: 865 - DNA, 51 bp, Homo sapiens
attctagaacacttctcaataccttwtctaaatgccaatctctatccaagg

SEQ ID NO: 866 - DNA, 51 bp, Homo sapiens
caaactataaatagagaactaaaaawtcctaaatctgatattgggcatcta

SEQ ID NO: 867 - DNA, 51 bp, Homo sapiens
ttaacaatattcactctttttataartgatcatggcatgtttttccttttt

SEQ ID NO: 868 - DNA, 51 bp, Homo sapiens
aaagaaattagagatgatacacacayaaaaaaggaaaaacatgccatgatc

SEQ ID NO: 869 - DNA, 51 bp, Homo sapiens
atcaaaaacataaaatcaaaggattsttgtcttcattattattaatctaga

SEQ ID NO: 870 - DNA, 51 bp, Homo sapiens
tagaggcgatttaaagtatatgagaraaagtggatgtgtgtaggctatatg

SEQ ID NO: 871 - DNA, 51 bp, Homo sapiens
ctctgaccacagctccagagggcccraagcaatatgtggtattgtggccac

SEQ ID NO: 872 - DNA, 51 bp, Homo sapiens
tctcaacaattaggttaacaacaatmctggaagctgcaagagatgtgatct

SEQ ID NO: 873 - DNA, 51 bp, Homo sapiens
tctagaactctaaggaggattaggtyaaaaccaacagcatagacagaattt

SEQ ID NO: 874 - DNA, 51 bp, Homo sapiens
cctaagacaaagatagatgtttggaygaccatgaaaatttgagaggcccaa

SEQ ID NO: 875 - DNA, 51 bp, Homo sapiens
gagatagggtattcaaagtggtatcyagttaaaatgaattcattagggttg

SEQ ID NO: 876 - DNA, 51 bp, Homo sapiens
cctatttattcacttgagcattctgratatcattgacattttctttgactg

SEQ ID NO: 877 - DNA, 51 bp, Homo sapiens
aacattacttcccaaatcagattaarccaaagggggaacagaccttcacaa

SEQ ID NO: 878 - DNA, 51 bp, Homo sapiens
gcctttcactattcctttaccagccrcagaaataaaaaataattgaaagtt

SEQ ID NO: 879 - DNA, 51 bp, Homo sapiens
atggagaattagtaaaattagagtaygtcatcgtggattaggtatgattta

SEQ ID NO: 880 - DNA, 51 bp, Homo sapiens
gcatacttgctccaaggtttaaatartataatattgcatgtcaagtgcttt

SEQ ID NO: 881 - DNA, 51 bp, Homo sapiens
tttgtggtctgtaccttttgacatawactatgctgaacacatggcctttcc

SEQ ID NO: 882 - DNA, 51 bp, Homo sapiens
acagattaaaaatggggtaagtctaygcaaactaacttagaaaagtattca

SEQ ID NO: 883 - DNA, 51 bp, Homo sapiens
agagatttcttactagataagagtgmaataaacccttgaaaatcaacttca

SEQ ID NO: 884 - DNA, 51 bp, Homo sapiens
cctataatcagaggctaaaatggtcrataaccaaaattatcatcaaccttc

SEQ ID NO: 885 - DNA, 51 bp, Homo sapiens
ccacaagagttttaattctattacasaggagtagtcctacaaactaatgta

SEQ ID NO: 886 - DNA, 51 bp, Homo sapiens
cctggcccagctgccttaacttagcrcaaatgcaaacttgccctgtatctc

SEQ ID NO: 887 - DNA, 51 bp, Homo sapiens
ggatttgggaaatgtgcgttaaaaaygttctgtgtgaggcagagaagtcat

SEQ ID NO: 888 - DNA, 51 bp, Homo sapiens
cagaaaagaaaccacaatcttgtaaygaggtggtgagactaaatcagcaaa

SEQ ID NO: 889 - DNA, 51 bp, Homo sapiens
gagtttcattatagacaattgggctsagaagaccctgaataaatcttattt

SEQ ID NO: 890 - DNA, 51 bp, Homo sapiens
acagagcagaagagcaggctggctaygcattcttaacgagtctgcctctag

SEQ ID NO: 891 - DNA, 51 bp, Homo sapiens
atctttcttatctttgcacaatcaaracttataaaagcctcagggaactga

SEQ ID NO: 892 - DNA, 51 bp, Homo sapiens
cctgtttggttttctcctccttggcraacacatgtctcctggaacacagcg

SEQ ID NO: 893 - DNA, 51 bp, Homo sapiens
ttagcaatacacttttcattagtgarcttttttagtaagatgacattgaaa

SEQ ID NO: 894 - DNA, 51 bp, Homo sapiens
ccaagcttgtaaagtctcccgtggayatcttaattcttatattttcctttc

SEQ ID NO: 895 - DNA, 51 bp, Homo sapiens
gcagattaaaacagagtaaggcacaragagtttcaatgacctgttcaagtc

SEQ ID NO: 896 - DNA, 51 bp, Homo sapiens
atctctcagcttataaggacagctcygaaattaattacaaaacaaaacctc

SEQ ID NO: 897 - DNA, 51 bp, Homo sapiens
ccacagcatctatgcagattaggacsagccagtgggaagaaagcgattagt

SEQ ID NO: 898 - DNA, 51 bp, Homo sapiens
gcatttccactaacttagtgttttgmaaacttgtctatatcttggaatgtt

SEQ ID NO: 899 - DNA, 51 bp, Homo sapiens
atccatgccagggcacagaggattayagaataaaggctacgtggttaacag

SEQ ID NO: 900 - DNA, 51 bp, Homo sapiens
ttagaaaaaaatactctattagtcaygtggaggatggtggcaagactaaga

SEQ ID NO: 901 - DNA, 51 bp, Homo sapiens
cttatggccagatgacgtcaactctyactataaaagatgatgttttccatt

SEQ ID NO: 902 - DNA, 51 bp, Homo sapiens
tttaacataactaaaagaatataacygcattgactgtaacacaaaggatgg

SEQ ID NO: 903 - DNA, 51 bp, Homo sapiens
taatattttttgtttgcttaggaccrgagcaagaaggttggacttgggagc

SEQ ID NO: 904 - DNA, 51 bp, Homo sapiens
ttggttcaggcattttttccacacasagttcagaaatatactatggaaaga

SEQ ID NO: 905 - DNA, 51 bp, Homo sapiens
tatgttgttatgtgtgagtttggcaraaatgtagtagagtaacccagatga

SEQ ID NO: 906 - DNA, 51 bp, Homo sapiens
agatgaattacagacacctttaggayatagaatcaatagttcttgataatg

SEQ ID NO: 907 - DNA, 51 bp, Homo sapiens
ggtcattttattgttttgttttgaastcaagtggcagacatagacactggc

SEQ ID NO: 908 - DNA, 51 bp, Homo sapiens
gtttcaaaacaatctgtctcatatcyactcccaaaagaaatcaggcataaa

SEQ ID NO: 909 - DNA, 51 bp, Homo sapiens
ggtgtagagagctgattaaaacacasatctgatcctgacaccctcttactc

SEQ ID NO: 910 - DNA, 51 bp, Homo sapiens
agccctcctggcctggacttcacaasatgagtaagagggtgtcaggatcag

SEQ ID NO: 911 - DNA, 51 bp, Homo sapiens
tggctggcagaaagctgggaaagtasgctagtcctggcagccctcctggcc

SEQ ID NO: 912 - DNA, 51 bp, Homo sapiens
tacttgaacccaacttatgcttaaakattgaaacatttaccaaatatcttc

SEQ ID NO: 913 - DNA, 51 bp, Homo sapiens
tgtttttttaaaaaatcaagctatawagaagtgtatagagtaaaatacata

SEQ ID NO: 914 - DNA, 51 bp, Homo sapiens
tctgtcaaaatttacctaagaagaartacaccatctgaattttctattaaa

SEQ ID NO: 915 - DNA, 51 bp, Homo sapiens
gaacagggaagtggaataagaggccrctacgaagtgagaggtagcttccag

SEQ ID NO: 916 - DNA, 51 bp, Homo sapiens
tctagtagggaatatgagacattgaygtatgacaattgttatagaataaaa

SEQ ID NO: 917 - DNA, 51 bp, Homo sapiens
aatgccatccagatgtctagatgcayatccacattaacatattatgcaaat

SEQ ID NO: 918 - DNA, 51 bp, Homo sapiens
tagaaacagcacttggattttaaaamcttttcacctatatagatggtttca

SEQ ID NO: 919 - DNA, 51 bp, Homo sapiens
ctctgaggctgccaaggaattatgcraaggacttatgaatccatgtacaca

SEQ ID NO: 920 - DNA, 51 bp, Homo sapiens
aaataatttgaacattcctaactcawgcttattggaattttatgactttct

SEQ ID NO: 921 - DNA, 51 bp, Homo sapiens
tttaaaaggcttaaactagagatcarctcatgataagcactaatgaatgtt

SEQ ID NO: 922 - DNA, 51 bp, Homo sapiens
acttagcatgccagaaagttcgaaarcaaaggctctggaatcagaaatgtc

SEQ ID NO: 923 - DNA, 51 bp, Homo sapiens
tcaagtgtagccagtagtccttccaracgagaatgtaacatggaggcatgg

SEQ ID NO: 924 - DNA, 51 bp, Homo sapiens
gatctagcttcttccagttctgaagycctctgatttgatgtctggaaagct

SEQ ID NO: 925 - DNA, 51 bp, Homo sapiens
ccttgattcaagacctgagtatggcrcactcatcactggtcaggcacaagg

SEQ ID NO: 926 - DNA, 51 bp, Homo sapiens
atgactctccatcaaattaaattgcwtgttcttggaggaggggggaggtaa

SEQ ID NO: 927 - DNA, 51 bp, Homo sapiens
ttaggcatccaagggtcttcgggcaytatttcagaaggcgtctcaagctct

SEQ ID NO: 928 - DNA, 51 bp, Homo sapiens
aaaaggcgcatagtctatggactagraagtactgggatgtttcacatgaag

SEQ ID NO: 929 - DNA, 51 bp, Homo sapiens
atattttagcatttatgtatctctasactgtaatgaattgacagtttactt

SEQ ID NO: 930 - DNA, 51 bp, Homo sapiens
aacaagatttcttcttgcttaagatwactacaaaagcagttttgcaacaaa

SEQ ID NO: 931 - DNA, 51 bp, Homo sapiens
gttaaaatatgagaggtatggcttasaggaatattaaaaatgaatggaaca

SEQ ID NO: 932 - DNA, 51 bp, Homo sapiens
atagagttgaagtttaaaactgatcraaaactgaatactagaatggttttg

SEQ ID NO: 933 - DNA, 51 bp, Homo sapiens
tggcagcaaaaacaaaaaagaaagayctgctttataagttacttccttgta

SEQ ID NO: 934 - DNA, 51 bp, Homo sapiens
caggccttttggtatgaaaagactawaaattacaggacacaagcattcatt

SEQ ID NO: 935 - DNA, 51 bp, Homo sapiens
gagtactatggcattaaaaatgatarcatccagtaaatagctccatctata

SEQ ID NO: 936 - DNA, 51 bp, Homo sapiens
aagactgtgcactcttgggtggaaaygaaattatatagaaagaaaagattc

SEQ ID NO: 937 - DNA, 51 bp, Homo sapiens
ctgatcaatgggaaaaaaaactgaastcagccatgaataatatgtatgcca

SEQ ID NO: 938 - DNA, 51 bp, Homo sapiens
agaaagacagggagagtctccaggarctataagaatgtaatcaacttgagc

SEQ ID NO: 939 - DNA, 51 bp, Homo sapiens
aaggaatgaattttttgtgtgataargtcaaacagcacgtggaattagcca

SEQ ID NO: 940 - DNA, 51 bp, Homo sapiens
ttgtctctaaaagccaattggtttaygcatttattcaataaacacttattg

SEQ ID NO: 941 - DNA, 51 bp, Homo sapiens
tcacatagcataggctataacttcaytaaagaaaacatggtcacccctgcc

SEQ ID NO: 942 - DNA, 51 bp, Homo sapiens
tggcttctttactcccatataaaackaacaatgatcatagaaaatgtgtca

SEQ ID NO: 943 - DNA, 51 bp, Homo sapiens
gccttatctacaatataataatttcycacaaatgcaaagaatataggtttg

SEQ ID NO: 944 - DNA, 51 bp, Homo sapiens
gggactataggcacctgctactgtayctggctaaaatgttttttaagctgg

SEQ ID NO: 945 - DNA, 51 bp, Homo sapiens
tgtcaactgagtatacactggagacygaaaccatgatgaagtcagttaaaa

SEQ ID NO: 946 - DNA, 51 bp, Homo sapiens
ggagagccttcaagttatgattaggwatgacaattgtgaaagaagagaagg

SEQ ID NO: 947 - DNA, 51 bp, Homo sapiens
ttagaaataatattttttgcagccaygtaaattgtttcatcttctatttat

SEQ ID NO: 948 - DNA, 51 bp, Homo sapiens
agtagggtcttgaagtctaagaaagwattgtcattgtctgatttgactccc

SEQ ID NO: 949 - DNA, 51 bp, Homo sapiens
tggaatatacatacagaaacacaaawtgagggtgacaaaagtgtcaaactg

SEQ ID NO: 950 - DNA, 51 bp, Homo sapiens
tccaattgatttgtcaaagaactacrtaagcttctgaaatagaaaaaaatt

SEQ ID NO: 951 - DNA, 51 bp, Homo sapiens
gagaaaagaggaaggggatggagagwcagaaacggagacttgttgtcaatg

SEQ ID NO: 952 - DNA, 51 bp, Homo sapiens
gggaatagtggtgatagtctgactgkaatccccatgggcctgtggtcgtgg

SEQ ID NO: 953 - DNA, 51 bp, Homo sapiens
actctaatacaagggacgtatcacarcagagcatgtgtgaaatgattattt

SEQ ID NO: 954 - DNA, 51 bp, Homo sapiens
ccacaaaggacgatgagatttacaargagttttcccagaggcatgacgttt

SEQ ID NO: 955 - DNA, 51 bp, Homo sapiens
atcttgaaattccctaagtccaatarctccgtgccaggtcgttttaggggt

SEQ ID NO: 956 - DNA, 51 bp, Homo sapiens
ccatgtttctaaagctgaaaatctcrctagtctacaaaaatgacatcagga

SEQ ID NO: 957 - DNA, 51 bp, Homo sapiens
ttggttcaacgaaagataccaaagayaaagttaacagacgggtgatagaga

SEQ ID NO: 958 - DNA, 51 bp, Homo sapiens
aagaatttccattatgaatgttggaytcagtcatggacatttacatgaatc

SEQ ID NO: 959 - DNA, 51 bp, Homo sapiens
tgaagattagcacaaaaaagagtaamtcaagccttgggtgaacagaatgtc

SEQ ID NO: 960 - DNA, 51 bp, Homo sapiens
cactctccctttgccactgcactagyaaacgtttgtgaaaagaatctgcag

SEQ ID NO: 961 - DNA, 51 bp, Homo sapiens
tctagaactcagctgtcgtgagagasaagtgtaagcctgtgggtaggagca

SEQ ID NO: 962 - DNA, 51 bp, Homo sapiens
ggactttatcccaggtgtagcaccaytgagaggttttcaacagggaaatga

SEQ ID NO: 963 - DNA, 51 bp, Homo sapiens
aactttttaatgacaaacaatactasagagttacaaaaatatttttagatg

SEQ ID NO: 964 - DNA, 51 bp, Homo sapiens
ttgtgatctcctcacacagaggctaytaagagttggcagactgcattatca

SEQ ID NO: 965 - DNA, 51 bp, Homo sapiens
tgtatttttgtgtgtaattctttaayccatctggagatattttggttgttt

SEQ ID NO: 966 - DNA, 51 bp, Homo sapiens
caactagctacatagggaagcagtayagcacagtacattttttggtcttca

SEQ ID NO: 967 - DNA, 51 bp, Homo sapiens
ctgtatatctttctctttgtcaaatyaatatccaaacactctacttgtttt

SEQ ID NO: 968 - DNA, 51 bp, Homo sapiens
ttgaagtagcaaccaaagctgtccaraaacttgttcagcatatatgcctta

SEQ ID NO: 969 - DNA, 51 bp, Homo sapiens
ttgttttatgccaagcactttgttaygtgttgaggataagccgcttggttc

SEQ ID NO: 970 - DNA, 51 bp, Homo sapiens
caatacatctcagcatattctatgarcaatacatctcagctgctgtctgag

SEQ ID NO: 971 - DNA, 51 bp, Homo sapiens
tgcacaagcattgtctataacttcawctgaacacttgctctaccttacaca

SEQ ID NO: 972 - DNA, 51 bp, Homo sapiens
gggacttcaaacctagatctacctaycaaaacattataccaatccctcatg

SEQ ID NO: 973 - DNA, 51 bp, Homo sapiens
ttgaaataatcgaaatgggaaaaacmgaacttatccataacgcctccccac

SEQ ID NO: 974 - DNA, 51 bp, Homo sapiens
ttgatctgtaatcttccaaggtcacrctaaggttgataagtatacttgagt

SEQ ID NO: 975 - DNA, 51 bp, Homo sapiens
cttggaatcgaaggggagaccaggarcataaagaacacactggaaaggtgt

SEQ ID NO: 976 - DNA, 51 bp, Homo sapiens
cattttttacctcactcaagggtcayagtaattattaaaacagtcatcggc

SEQ ID NO: 977 - DNA, 51 bp, Homo sapiens
catccagggaggaagcaatgataagrctctaggaggtggcattcacctctg

SEQ ID NO: 978 - DNA, 51 bp, Homo sapiens
gtagttagaatagctctccacaataytaactaactaactaaataaataaat

SEQ ID NO: 979 - DNA, 51 bp, Homo sapiens
ccaataccaaagacattgaagctaayaatatcttctcctccacttgctgaa

SEQ ID NO: 980 - DNA, 51 bp, Homo sapiens
gatcttcaacatgcattccaaggacrcaaaacagggaaattattgtttctt

SEQ ID NO: 981 - DNA, 51 bp, Homo sapiens
atattcaaaatactattgctaagacrctaagaccaacatcaagaaggtttt

SEQ ID NO: 982 - DNA, 51 bp, Homo sapiens
ttctcaggccattattccatccctcratccattttctactactttccctgc

SEQ ID NO: 983 - DNA, 51 bp, Homo sapiens
aaagtgcacttatgtgctatacttcratacaaatttactttaaaaaagcag

SEQ ID NO: 984 - DNA, 51 bp, Homo sapiens
cctggagagggcaggttagatttgargaacttataccttagtgtttttttt

SEQ ID NO: 985 - DNA, 51 bp, Homo sapiens
ctctcttctttatttgctgtaaattyctttcagggtcatgccagctagcca

SEQ ID NO: 986 - DNA, 51 bp, Homo sapiens
attaaaaatgccttaaactttagacwatataccaattcaatgcttttaatt

SEQ ID NO: 987 - DNA, 51 bp, Homo sapiens
attctagttcactttagtgtgatagyacatacatctaatgagtatcatatg

SEQ ID NO: 988 - DNA, 51 bp, Homo sapiens
caatccagctggtatgataaagaccrggtcagcccaggagaatctctcaaa

SEQ ID NO: 989 - DNA, 51 bp, Homo sapiens
caggaagagtactgataagtagaggrcagctgaaccaaggcccaagcaccc

SEQ ID NO: 990 - DNA, 51 bp, Homo sapiens
agcaggcagttatgtgttttcaagamagtaatttaccaagttgaaattcct

SEQ ID NO: 991 - DNA, 51 bp, Homo sapiens
gccaccacagtgggccccaggagatyagaagattagaattcctcacaaacc

SEQ ID NO: 992 - DNA, 51 bp, Homo sapiens
tttattagttttcaatcacacttcaraatgatgcaggtcacagtgtgtcta

SEQ ID NO: 993 - DNA, 51 bp, Homo sapiens
ttcacatagtaaagtgagaaattaaygaatctaaagagaaaaagcaaataa

SEQ ID NO: 994 - DNA, 51 bp, Homo sapiens
tttcaactcagaggcaaagaaataasatggcaggatatagtgaatgaaaca

SEQ ID NO: 995 - DNA, 51 bp, Homo sapiens
aactgacgtgatgctaagcagaaccrgaaagtaaaacagaaaggatatggg

SEQ ID NO: 996 - DNA, 51 bp, Homo sapiens
cttatgagaattttctgtatggacartggaatattgtgttaggcgtttagg

SEQ ID NO: 997 - DNA, 51 bp, Homo sapiens
aggtaggtataaatgaataaaacaascaaattgcaagcagaaataacagtg

SEQ ID NO: 998 - DNA, 51 bp, Homo sapiens
ggagaaatccaagactctgaaggaaygaataatgtttcttaaatatctctg

SEQ ID NO: 999 - DNA, 51 bp, Homo sapiens
ttgacttccaacttttaattttgacygtaataattatatactttctctaaa

SEQ ID NO: 1000 - DNA, 51 bp, Homo sapiens
ttttgcatagcagacacaggaacctragatattaaccctcatctactttct

SEQ ID NO: 1001 - DNA, 51 bp, Homo sapiens
gcaagagataatttcgggtgatccaraaatgcatgtcaggttgtgtgtcac

SEQ ID NO: 1002 - DNA, 51 bp, Homo sapiens
tattgagattttgggtaagcatgagraccgcagtacataccccgtcttcat

SEQ ID NO: 1003 - DNA, 51 bp, Homo sapiens
acaattgtttgatgcttaatcttgayattaaaacctaaaacactactttca

SEQ ID NO: 1004 - DNA, 51 bp, Homo sapiens
acattctaatattgcatagccatgaragctgggaaaatgctgctgttttag

SEQ ID NO: 1005 - DNA, 51 bp, Homo sapiens
gtagaagcgtttgtagtagagagcaytgactccagatctcattttcttctt

SEQ ID NO: 1006 - DNA, 51 bp, Homo sapiens
gagtataatgtattcatgaaagttamggatttttgacaaatggaataatat

SEQ ID NO: 1007 - DNA, 51 bp, Homo sapiens
aagtaccatgagttaagaagtaacartataaactaaagtcttaaattggga

SEQ ID NO: 1008 - DNA, 51 bp, Homo sapiens
ccgtcaagccagatgatggtaggtayatttagcaacaaagttctatacaaa

SEQ ID NO: 1009 - DNA, 51 bp, Homo sapiens
taaatgttcttcctatttgttcttawatcaattttgttaaagaatatcaga

SEQ ID NO: 1010 - DNA, 51 bp, Homo sapiens
aaatcaattttgttaaagaatatcasaaactttatagacgtgattaaatct

SEQ ID NO: 1011 - DNA, 51 bp, Homo sapiens
tcttttaaagtccctgtgtccccaaytacaactgaactgggtttaaaaaaa

SEQ ID NO: 1012 - DNA, 51 bp, Homo sapiens
cacgtgtgaaagcgtgcagcatacargccagtgtgtagttgaaaattgcag

SEQ ID NO: 1013 - DNA, 51 bp, Homo sapiens
aaatatttaagaacactacggcctayatctataacataaaagtattttctg

SEQ ID NO: 1014 - DNA, 51 bp, Homo sapiens
gggccttgtgctcagtgacagaccasagtcccatcactgagagagcagggc

SEQ ID NO: 1015 - DNA, 51 bp, Homo sapiens
aagttaaatcacagataaggaaataygtagaagacaagggaaagatgagga

SEQ ID NO: 1016 - DNA, 51 bp, Homo sapiens
gcttgccatctaatccgtaaggaacscagtcacagagcaatgtaaaggaag

SEQ ID NO: 1017 - DNA, 51 bp, Homo sapiens
gtatcaacacaaccttcccagatcayaacggcaaggggagaagatgctaga

SEQ ID NO: 1018 - DNA, 51 bp, Homo sapiens
ttattatgaaagacaatgaagcccayggaagctgtagaatttacaaggtgg

SEQ ID NO: 1019 - DNA, 51 bp, Homo sapiens
gggatcatccagtggagttgtaaacragggaaccatcttgcaagatctagg

SEQ ID NO: 1020 - DNA, 51 bp, Homo sapiens
cttcaggcagataaaggtatacactsaatttttcattctctaggacttcat

SEQ ID NO: 1021 - DNA, 51 bp, Homo sapiens
tgctgaaggttgatagtaaccaaaaygcttttcagagaactaggtcacaca

SEQ ID NO: 1022 - DNA, 51 bp, Homo sapiens
aaatgctaattgttccagtaattagraagttctctggacaaagcccaaggt

SEQ ID NO: 1023 - DNA, 51 bp, Homo sapiens
gggaagagccagtggaggaggaagayggcaaactcgcctgtgaagaggctt

SEQ ID NO: 1024 - DNA, 51 bp, Homo sapiens
tttcttcatctatggaggactgcaaycattatcattatttcccagaaccta

SEQ ID NO: 1025 - DNA, 51 bp, Homo sapiens
gcttaaagggtgtatttatcttgctkagttctagtcgcagataacttaaca

SEQ ID NO: 1026 - DNA, 51 bp, Homo sapiens
tttttcttgcctcttctgctagaaaktcagcatcttacaagcagatggtgt

SEQ ID NO: 1027 - DNA, 51 bp, Homo sapiens
aattcagcttttggattaaatcaagyattgaatggataatttatccccaat

SEQ ID NO: 1028 - DNA, 51 bp, Homo sapiens
atgtttaattgtattccttttaacaktcctttaaccatattccttttcata

SEQ ID NO: 1029 - DNA, 51 bp, Homo sapiens
tccagatttagatgtcaaagcccagmatgaaaagagcatgtgtgatttgta

SEQ ID NO: 1030 - DNA, 51 bp, Homo sapiens
gtcaaggggttctgtacttctgtagycattactttggaatgggctattcca

SEQ ID NO: 1031 - DNA, 51 bp, Homo sapiens
tcaccagtatttactaaataaatgcycagaaaaaaaatagttcatctttaa

SEQ ID NO: 1032 - DNA, 51 bp, Homo sapiens
gatctatcttctttcaactacctcayacaacctctctgttaaagcttacct

SEQ ID NO: 1033 - DNA, 51 bp, Homo sapiens
cctcagtgtcctcaaatgtaaactamggatggcatccctcctgggccatta

SEQ ID NO: 1034 - DNA, 51 bp, Homo sapiens
tttcaagactctggacaaaacatacracaaactgaggagcactaattcatt

SEQ ID NO: 1035 - DNA, 51 bp, Homo sapiens
tgacttttgacacaagatgatcggayatttgtgcggtggattttcaaaggt

SEQ ID NO: 1036 - DNA, 51 bp, Homo sapiens
ctggaaatgattttctgatttaaaamggaggacaatcaactcgatagtagg

SEQ ID NO: 1037 - DNA, 51 bp, Homo sapiens
atagcattcatatcaatgttgcatamatacaggaaaatatacaacccaata

SEQ ID NO: 1038 - DNA, 51 bp, Homo sapiens
gcaaaggattattttagaatccataygtagagtgcccaaaaggaaaagctt

SEQ ID NO: 1039 - DNA, 51 bp, Homo sapiens
tctctggaaagtgatcaaaggcttayaacatctgaagaactgaaaaactgc

SEQ ID NO: 1040 - DNA, 51 bp, Homo sapiens
aggcagactttattcaagagagctayggtgatagggtcaccaccatggggt

SEQ ID NO: 1041 - DNA, 51 bp, Homo sapiens
gttaaactttaaaaataaattctcaractacgagatagatatatatatttt

SEQ ID NO: 1042 - DNA, 51 bp, Homo sapiens
ttaaagatcccccaaaatgtgactasataaaattgatcatgcacaatccat

SEQ ID NO: 1043 - DNA, 51 bp, Homo sapiens
tccttaagggacacttggaaaataawctcagccttatgaatggccaaactc

SEQ ID NO: 1044 - DNA, 51 bp, Homo sapiens
aaaatatctagagacattggaggacmactatctataaaattaaggcataga

SEQ ID NO: 1045 - DNA, 51 bp, Homo sapiens
agtgatgatggcagggatcacaggckgattatataaagttcttagtgtttt

SEQ ID NO: 1046 - DNA, 51 bp, Homo sapiens
tgccagattcagtattacacattccrgatgcctgagcttttgcataatgat

SEQ ID NO: 1047 - DNA, 51 bp, Homo sapiens
gaaacatagactattatgtaaacaartatctcaaataactcaatatatagt

SEQ ID NO: 1048 - DNA, 51 bp, Homo sapiens
ttatctactttatttcatttcatacrctcagcaacctttgaggtagataca

SEQ ID NO: 1049 - DNA, 51 bp, Homo sapiens
agtcaagaaagaaaagaaagatgtaygttgctatcaagttttaattgaagc

SEQ ID NO: 1050 - DNA, 51 bp, Homo sapiens
agtgttatgaaagaggaggtctaatsaagtacaaacactcagagaattcta

SEQ ID NO: 1051 - DNA, 51 bp, Homo sapiens
tcctataacccagagcttatcagagsatttatgtccccaaagagaaatgtc

SEQ ID NO: 1052 - DNA, 51 bp, Homo sapiens
catttttaaagcttaaagaaagaaasttgtgcaactgtcaaaacgtcatca

SEQ ID NO: 1053 - DNA, 51 bp, Homo sapiens
atggaaaaagtcctctaagccaccaytgggagtacaaatcattactttata

SEQ ID NO: 1054 - DNA, 51 bp, Homo sapiens
catccaaaccataccagtgagtttayacttacattgaaactggtgaagagg

SEQ ID NO: 1055 - DNA, 51 bp, Homo sapiens
tacccagcattctattgctcccaggraaaagaccaggaaggatgctctggc

SEQ ID NO: 1056 - DNA, 51 bp, Homo sapiens
ccaagaatttgggaaagggcatgaaygtgtaaactgagtactacagatctg

SEQ ID NO: 1057 - DNA, 51 bp, Homo sapiens
aacagtgctgtatgaagattataccratgtacacagccaacagccatgtac

SEQ ID NO: 1058 - DNA, 51 bp, Homo sapiens
ttttctctgcgctcgggaataataaytcggaatgacccaaggaaccagagg

SEQ ID NO: 1059 - DNA, 51 bp, Homo sapiens
ttaaacagaacatgtcagatcttaaytcctctgactttatgactacgctca

SEQ ID NO: 1060 - DNA, 51 bp, Homo sapiens
tgactttttcttccaaataattttarcaatgctcacgctttctggtattgc

SEQ ID NO: 1061 - DNA, 51 bp, Homo sapiens
tacatagaaaactattagctctccargatttaattctactcaactctaatc

SEQ ID NO: 1062 - DNA, 51 bp, Homo sapiens
ctagtggacttcactcgccagcataygggcaatgtaaactctgcccttccc

SEQ ID NO: 1063 - DNA, 51 bp, Homo sapiens
tcccaggccaacagatgcaaggcaawtgattcagtcgcttactccagcccc

SEQ ID NO: 1064 - DNA, 51 bp, Homo sapiens
cttaactccaaactccatcctttgargttaagattagctgaggactcttgg

SEQ ID NO: 1065 - DNA, 51 bp, Homo sapiens
gttgaggggaagtatctttctgggcrctattatatacaatacttcaaagtt

SEQ ID NO: 1066 - DNA, 51 bp, Homo sapiens
tttcttttttaataacggaattaaartgcttacctaactaacacccacata

SEQ ID NO: 1067 - DNA, 51 bp, Homo sapiens
gctctgaaagataacaaacaggcaamatcttttacttcttgctcacaagta

SEQ ID NO: 1068 - DNA, 51 bp, Homo sapiens
ctagggaacattctcaggaaaatcaytattggtttctacaacagtgtcttc

SEQ ID NO: 1069 - DNA, 51 bp, Homo sapiens
actaatctctccttaagtgagaagaracagtgatcatgagcttggacatga

SEQ ID NO: 1070 - DNA, 51 bp, Homo sapiens
aattgtatcctctgttgtacataatraaacaagcggtttggcgtgaggcgt

SEQ ID NO: 1071 - DNA, 51 bp, Homo sapiens
actggggaaagataatgatggaatayacctgggagagctgtaggagaagga

SEQ ID NO: 1072 - DNA, 51 bp, Homo sapiens
gaatccacacttactgcctactttaygccttagggatccttgagaatttcc

SEQ ID NO: 1073 - DNA, 51 bp, Homo sapiens
ataaaatatcatcaggctgctacaasaactttcattgtcatcacccctcat

SEQ ID NO: 1074 - DNA, 51 bp, Homo sapiens
taaactagatgttgtctttttgtaartattttagcaacatgtatgtatgtt

SEQ ID NO: 1075 - DNA, 51 bp, Homo sapiens
tttggtatcatggaataaggaatccrgtaacttgaaggcagatagaaatta

SEQ ID NO: 1076 - DNA, 51 bp, Homo sapiens
tgaattttagaactagaagtgtgcaygtatgtcaaaagattatgcagagct

SEQ ID NO: 1077 - DNA, 51 bp, Homo sapiens
gactctctttgcacttttagcttaasgtctaggctaggaaatgcctcagaa

SEQ ID NO: 1078 - DNA, 51 bp, Homo sapiens
ataattgcttataatacacagactcrattttatatgactaattagaacagt

SEQ ID NO: 1079 - DNA, 51 bp, Homo sapiens
ggagtcatacccattgactttctgckctcctggatgagatttaagtctcta

SEQ ID NO: 1080 - DNA, 51 bp, Homo sapiens
agactaatgatacagttctaagacasggtgtttggctttaaaaacaacaat

SEQ ID NO: 1081 - DNA, 51 bp, Homo sapiens
acacttaaaccaccactgagtccaaygatttccacatcagtggggaggcac

SEQ ID NO: 1082 - DNA, 51 bp, Homo sapiens
gggcattcattctgaggtttttaagyccttcaattcttcatccctattcct

SEQ ID NO: 1083 - DNA, 51 bp, Homo sapiens
aaaatccagataattgaggctaaaamattttctatggtaatttgtcagaat

SEQ ID NO: 1084 - DNA, 51 bp, Homo sapiens
ctgtagggctagagttctacactacrcatttgttacttgctgtgtactgtg

SEQ ID NO: 1085 - DNA, 51 bp, Homo sapiens
gaattaaatttttttggctgactccrcaattttacctacttcacaacgata

SEQ ID NO: 1086 - DNA, 51 bp, Homo sapiens
atgaaaattccttcttggtggctagmaagtttgaaaatttctgaatttatt

SEQ ID NO: 1087 - DNA, 51 bp, Homo sapiens
atcatatttagtatattatttgatakgtatgataaagttcagaaaagctta

SEQ ID NO: 1088 - DNA, 51 bp, Homo sapiens
acttgaagacacaaaagcagtcaacygagctataaaggaaaactaaagaat

SEQ ID NO: 1089 - DNA, 51 bp, Homo sapiens
taagtgaaatcaaatgagagaagtcyaaccttgcaaaactgagccaactgc

SEQ ID NO: 1090 - DNA, 51 bp, Homo sapiens
gtcttctagatgcaattgttttctayagtctgtcttgtggccggttataaa

SEQ ID NO: 1091 - DNA, 51 bp, Homo sapiens
cttcacaaatgacttaaggcaactcrctttcgggttgatggaagaaaaaat

SEQ ID NO: 1092 - DNA, 51 bp, Homo sapiens
cccataggccagtgaaatggatccartaagacataactaacttcagtttct

SEQ ID NO: 1093 - DNA, 51 bp, Homo sapiens
gtgaaatgaaaatcatttgtacgaaytgactaacgaggattattatatttt

SEQ ID NO: 1094 - DNA, 51 bp, Homo sapiens
tcttgaatcccattgctctgctagayagatttaaatagtagtgtaagaaat

SEQ ID NO: 1095 - DNA, 51 bp, Homo sapiens
gcagctgcttaagccaatagaaattkaacatgcctaagtgcagcctcctgc

SEQ ID NO: 1096 - DNA, 51 bp, Homo sapiens
taaagacgtctgggtatcttcagtaygtaaaaagctgtgggaaaaaagagt

SEQ ID NO: 1097 - DNA, 51 bp, Homo sapiens
aaatctgagcattttgctagaacaaraaagaatagtgacattgagtttgac

SEQ ID NO: 1098 - DNA, 51 bp, Homo sapiens
tatttctcaaaaatgactgtgagggycaagtgttaaaagaaaaattctttt

SEQ ID NO: 1099 - DNA, 51 bp, Homo sapiens
aggagaagacaccagagatgcaaccrcacagagaaaaggtcatgtgagcag

SEQ ID NO: 1100 - DNA, 51 bp, Homo sapiens
gtgcatttctccctatagtggaaagmatactcagatctggtactactgccg

SEQ ID NO: 1101 - DNA, 51 bp, Homo sapiens
tggcctgtctccctttggacttctaygcgcgataaactatcttgttcaagc

SEQ ID NO: 1102 - DNA, 51 bp, Homo sapiens
ttttaaaacttcctttgttaatttayggaatactatttgattacttaataa

SEQ ID NO: 1103 - DNA, 51 bp, Homo sapiens
aatgtgtttgatgtgtttctcatgaytagaatttggttatgagtattggga

SEQ ID NO: 1104 - DNA, 51 bp, Homo sapiens
gaatgaaatggctgtggtgagaactraaattaacactatattgaaacaaat

SEQ ID NO: 1105 - DNA, 51 bp, Homo sapiens
tgttataaaggaaaatcataacatcraaggaagtaagtacagacttctcaa

SEQ ID NO: 1106 - DNA, 51 bp, Homo sapiens
gttggggtggttggaaatgacataamctgtgtgatgctttggatagttcta

SEQ ID NO: 1107 - DNA, 51 bp, Homo sapiens
actgtttttcttggtgaaataatcartacaaggtagttgcaagtttagaat

SEQ ID NO: 1108 - DNA, 51 bp, Homo sapiens
tctgtccaagtccttggctttcatcrgactctcaaaggagcctcagaaagc

SEQ ID NO: 1109 - DNA, 51 bp, Homo sapiens
tttttgtatactgatattcttgaacrccaactggataccctatcctactaa

SEQ ID NO: 1110 - DNA, 51 bp, Homo sapiens
gcttctgatggatatgcgaaagaacratcccattcagctgaaatacatgat

SEQ ID NO: 1111 - DNA, 51 bp, Homo sapiens
ataattcttttcaaccctggccttaygatgctaagggcacagttctcccct

SEQ ID NO: 1112 - DNA, 51 bp, Homo sapiens
gccatgctcacattctcctgctttayaagatgggaaaaaaagatattgtag

SEQ ID NO: 1113 - DNA, 51 bp, Homo sapiens
agaagtgataataaaaatagcaccaytctgaacagctggaaaccccggctt

SEQ ID NO: 1114 - DNA, 51 bp, Homo sapiens
aatggttactctataaagagattaaraaagaaagtcaagggagggtaagga

SEQ ID NO: 1115 - DNA, 51 bp, Homo sapiens
acaatgtgagatattcagagctttayatttagtaagggattcacttggttt

SEQ ID NO: 1116 - DNA, 51 bp, Homo sapiens
gagaatgggcaaaggaactatgctaygtcatgtcggaggatggataggtta

SEQ ID NO: 1117 - DNA, 51 bp, Homo sapiens
gaaccagcaatggaagattccaatayagcgataacacagaagtatcaaaat

SEQ ID NO: 1118 - DNA, 51 bp, Homo sapiens
ccctttatttttctttgtatactcaytgacacagccaaacttgtctattgt

SEQ ID NO: 1119 - DNA, 51 bp, Homo sapiens
aagaaaagcaaggcaaacaaatacasaaattttaagcaaataaaaggaaat

SEQ ID NO: 1120 - DNA, 51 bp, Homo sapiens
ggcttaagcaggaatgagcaatgctwacgctaacactggccatgatttttg

SEQ ID NO: 1121 - DNA, 51 bp, Homo sapiens
aattgcactatgactttatgccgtayacttcagtaagcagatatacaccca

SEQ ID NO: 1122 - DNA, 51 bp, Homo sapiens
taaatacttgtgtgcaaagacattcrctgcggctttacaacagtaaacaat

SEQ ID NO: 1123 - DNA, 51 bp, Homo sapiens
ctgctataacaaaatatcacactagycatttccacaactggctggcttata

SEQ ID NO: 1124 - DNA, 51 bp, Homo sapiens
taggaagatggaaaacactttagaaytgttaacatataatgatagtgacca

SEQ ID NO: 1125 - DNA, 51 bp, Homo sapiens
gaagaaaaagagggcgaatggagaaytatatgtcagagaattttatggatc

SEQ ID NO: 1126 - DNA, 51 bp, Homo sapiens
aaatgctagttgaataggatctggartacaggtttattaaaaaactaatta

SEQ ID NO: 1127 - DNA, 51 bp, Homo sapiens
aatgtctacatgacagagttatcaaratgaatgctacaatactttcaaaca

SEQ ID NO: 1128 - DNA, 51 bp, Homo sapiens
gtactcactctggcaccacagattcytataaacaccaaaggaagcaggttt

SEQ ID NO: 1129 - DNA, 51 bp, Homo sapiens
aagacaccgttaagagtgaaaaggcrattcatagaatggcataagatatat

SEQ ID NO: 1130 - DNA, 51 bp, Homo sapiens
tcctcccacatcccacagatgtacayattaggtgaattggctgtctacatt

SEQ ID NO: 1131 - DNA, 51 bp, Homo sapiens
ccatgcctttcatatccccagagatkagcaccaatttgcagacactgtcta

SEQ ID NO: 1132 - DNA, 51 bp, Homo sapiens
ctccccaggggactggctagcttaayagtcatctattcttaaaggccttaa

SEQ ID NO: 1133 - DNA, 51 bp, Homo sapiens
ttgtttattcagacaatggcacatayattggttaactgacttctacttaca

SEQ ID NO: 1134 - DNA, 51 bp, Homo sapiens
gtcagtgagtctaacatgaatccaasgcagaatatatttaataataaggct

SEQ ID NO: 1135 - DNA, 51 bp, Homo sapiens
atattggaaaagaagaaagatgtaarataaataatctaagcttctacatta

SEQ ID NO: 1136 - DNA, 51 bp, Homo sapiens
atgctgttccttctctttggaccaamcttcctccttcacttcacatggcta

SEQ ID NO: 1137 - DNA, 51 bp, Homo sapiens
gttcatttctacctcatggtatagartaacatccatgcagagacaaaaagg

SEQ ID NO: 1138 - DNA, 51 bp, Homo sapiens
ctcaagcaagagaagtacatgatgayggacctttgtactcaagtgccattt

SEQ ID NO: 1139 - DNA, 51 bp, Homo sapiens
gaacaaaacacagagtaatatcaaartatagaacgtaataggctagatgcc

SEQ ID NO: 1140 - DNA, 51 bp, Homo sapiens
ttcagcttaaaaaatgttcaccgtgkataaacctgtaatgagagctttcaa

SEQ ID NO: 1141 - DNA, 51 bp, Homo sapiens
gtgcagacacctaattggcatatcayataacaccccagaactcctttgctc

SEQ ID NO: 1142 - DNA, 51 bp, Homo sapiens
atgaggaatatgttaaagaaattgakgactttgcagaaataatgaaggtta

SEQ ID NO: 1143 - DNA, 51 bp, Homo sapiens
ggagatcaaagctctattttaggacractcactcaatgatgttgttcaggc

SEQ ID NO: 1144 - DNA, 51 bp, Homo sapiens
atgccacccatctcaagacttagacracaggcttgctcgggagaacttagt

SEQ ID NO: 1145 - DNA, 51 bp, Homo sapiens
gctaaatctcttggtgtccactgaartctttggtagcacaagaatatatgt

SEQ ID NO: 1146 - DNA, 51 bp, Homo sapiens
gaagtagtacatgttggctgaataamtgacccattcagcatcagctttggc

SEQ ID NO: 1147 - DNA, 51 bp, Homo sapiens
gcctgaccttccccatcctctaactyacaagatttcaaatgaaagtaggct

SEQ ID NO: 1148 - DNA, 51 bp, Homo sapiens
gaggctttgtaaactatctaaaatgwatctcttatctttaattgggaaaat

SEQ ID NO: 1149 - DNA, 51 bp, Homo sapiens
tcgagttttataccatttttgcttgyaagtataatctcctcaacctatttg

SEQ ID NO: 1150 - DNA, 51 bp, Homo sapiens
atgtaatttcaaccttcagagaaagrcattcttcctatcttacagataagg

SEQ ID NO: 1151 - DNA, 51 bp, Homo sapiens
atttttggaatttagacattaaaatrattgagtctggatttcattaccagg

SEQ ID NO: 1152 - DNA, 51 bp, Homo sapiens
gtgttggtagatttgtagatgtaaarttcccctcaatcatcttgaggacct

SEQ ID NO: 1153 - DNA, 51 bp, Homo sapiens
tcgtagttacctgtcaatggcaactyagttcataaatcttatctgctctgg

SEQ ID NO: 1154 - DNA, 51 bp, Homo sapiens
ttaaacattttctccaagatagatayaggatcctgcccaatttcagttaca

SEQ ID NO: 1155 - DNA, 51 bp, Homo sapiens
gtctatataaaacctgtttaatgtamatatttctctcactgaaaccatttt

SEQ ID NO: 1156 - DNA, 51 bp, Homo sapiens
gaatggtatataattaatgcattacyagaacacttaatagaactgtatcaa

SEQ ID NO: 1157 - DNA, 51 bp, Homo sapiens
caatcattgtttcttctttcaccagracccctgactgacaatcattgtgtc

SEQ ID NO: 1158 - DNA, 51 bp, Homo sapiens
cccggagaagagctacaacaggaaasgaatcagggtagcatcgtgcctctc

SEQ ID NO: 1159 - DNA, 51 bp, Homo sapiens
tatgcctgtttgtcacataacaatayagtcttcaaaaacttattccttgct

SEQ ID NO: 1160 - DNA, 51 bp, Homo sapiens
gaatagtccctgaggcttcagtaaaygcttcatgagttcagaaaaaatgag

SEQ ID NO: 1161 - DNA, 51 bp, Homo sapiens
catgcacagataaaaagaatattatyagacaaagtgaagactggatttctt

SEQ ID NO: 1162 - DNA, 51 bp, Homo sapiens
gccttatatcaccttatctattttaycaccttcatctcatattaacacaac

SEQ ID NO: 1163 - DNA, 51 bp, Homo sapiens
gcatgaatccattttcaccattcatkaagatccttcttttgctacttcact

SEQ ID NO: 1164 - DNA, 51 bp, Homo sapiens
gactaagattcccaggaactccttcragatactcctcttgatccaggtgct

SEQ ID NO: 1165 - DNA, 51 bp, Homo sapiens
aattttcctctcggttacatccaaasattcatatttttaacctgtctgaaa

SEQ ID NO: 1166 - DNA, 51 bp, Homo sapiens
ctagaccagacttttttaacaaccaytgcaccaaaaggaattttaagggct

SEQ ID NO: 1167 - DNA, 51 bp, Homo sapiens
ctcaaaggccatgattttgtgtcaawagatcagcaaataaatgcacattta

SEQ ID NO: 1168 - DNA, 51 bp, Homo sapiens
aagtaatagttgttggtagtaattgsatcataaaacctttcaaatgagttt

SEQ ID NO: 1169 - DNA, 51 bp, Homo sapiens
ggcaacagagaacgtgatgaagaacrctatggggaaagactcatctgctgt

SEQ ID NO: 1170 - DNA, 51 bp, Homo sapiens
tctattaggagtttgttttgctaaayggtatgacatgagattgatttttgt

SEQ ID NO: 1171 - DNA, 51 bp, Homo sapiens
cttgctttctcagactccctcaaaargacaggaacattttaattgtgtatc

SEQ ID NO: 1172 - DNA, 51 bp, Homo sapiens
catcttacggaagccaaagcaaagasgatggctatttttaacttgaggcag

SEQ ID NO: 1173 - DNA, 51 bp, Homo sapiens
ctaacctattcactcctaccataaakgattcaagaaaactggtgtgagttt

SEQ ID NO: 1174 - DNA, 51 bp, Homo sapiens
ttttcttaagcacatgagtgaataartgaattatatccccatagacagcat

SEQ ID NO: 1175 - DNA, 51 bp, Homo sapiens
ataaaagcaatgcttaatttagagaracactatcatgcattatacattcca

SEQ ID NO: 1176 - DNA, 51 bp, Homo sapiens
agaagttctccctggccagctttacraggaacctggtggcatttgatttca

SEQ ID NO: 1177 - DNA, 51 bp, Homo sapiens
acacaataacagaagttggttgttartaaatcacatcctagtctttcagca

SEQ ID NO: 1178 - DNA, 51 bp, Homo sapiens
tatgcagggatacaacagagatgagwaataaatattaaacaaactttattc

SEQ ID NO: 1179 - DNA, 51 bp, Homo sapiens
gatgggctcgtgctagggtgaactasaaaaccacatcattaacaatcctat

SEQ ID NO: 1180 - DNA, 51 bp, Homo sapiens
atgcttgcctcacacaaaatcatatsacatctcctcactgggtcttgaact

SEQ ID NO: 1181 - DNA, 51 bp, Homo sapiens
ggcccttttgtcgctatgaattttcygtctttaattctataaatgctttat

SEQ ID NO: 1182 - DNA, 51 bp, Homo sapiens
tattctagttgtttctttatgcaggyatttagtagaatctcacttgtcctt

SEQ ID NO: 1183 - DNA, 51 bp, Homo sapiens
ctcttgctaaaaatctgcctactacragagaatggccaatatgaataccac

SEQ ID NO: 1184 - DNA, 51 bp, Homo sapiens
cgttgtgaatgtgattaatacctctsaattgaacacttatgaaatggctaa

SEQ ID NO: 1185 - DNA, 51 bp, Homo sapiens
tggtgaagacttcaagttagtcctaygggaaggtcagtgcaagtgggaata

SEQ ID NO: 1186 - DNA, 51 bp, Homo sapiens
cctcaattatctggatttttccatayagatttttaaggccaactttatctg

SEQ ID NO: 1187 - DNA, 51 bp, Homo sapiens
tggtagagtcagaccagtggactaawaatagattgtaatgttgacatctag

SEQ ID NO: 1188 - DNA, 51 bp, Homo sapiens
atagggtcttagcttttaacttctaygcatgatcattaagaataaaagcta

SEQ ID NO: 1189 - DNA, 51 bp, Homo sapiens
gagattatgatgaagtatcaatagtsaaaatgttagagatttatagctatg

SEQ ID NO: 1190 - DNA, 51 bp, Homo sapiens
ccagagaaagtagcctcttgctctaygctaatggatttcttcaataggaga

SEQ ID NO: 1191 - DNA, 51 bp, Homo sapiens
atctagacttgtatttgactaaacarctgggtatcgtagcctagccaagtt

SEQ ID NO: 1192 - DNA, 51 bp, Homo sapiens
aagggctgctaggtggggagaaatcrgatagaatcaggtggggcagaggaa

SEQ ID NO: 1193 - DNA, 51 bp, Homo sapiens
ttagaaatgcccctgagacattgacsatggcttattcaaattaaagaaagc

SEQ ID NO: 1194 - DNA, 51 bp, Homo sapiens
ttttagcttaatgctttgcatccacyataataaacactggcttttctactg

SEQ ID NO: 1195 - DNA, 51 bp, Homo sapiens
ttctattgattgacttacagtaaagsatatcagaaaagaataatattgtcc

SEQ ID NO: 1196 - DNA, 51 bp, Homo sapiens
tgggattgttctttcgcatatccatsagaagcttttaaaattgtactgagg

SEQ ID NO: 1197 - DNA, 51 bp, Homo sapiens
ctactctttgttcagacagtagagayggcccttcttagaacagcagagatt

SEQ ID NO: 1198 - DNA, 51 bp, Homo sapiens
gtgtgggttctaaaccaaaaatcagraacacagactccctggcttgcagga

SEQ ID NO: 1199 - DNA, 51 bp, Homo sapiens
tcaagtgcctccaaagaaactcttaygattggagaaactggaaaatcactg

SEQ ID NO: 1200 - DNA, 51 bp, Homo sapiens
atatcctctgattctacatttagcartagatcccaacaatgttgacttgcc

SEQ ID NO: 1201 - DNA, 51 bp, Homo sapiens
tgcctaatttgttttagcatcaaaastcttcttttaaaagacatcttgaaa

SEQ ID NO: 1202 - DNA, 51 bp, Homo sapiens
agagtaaggtaccatggaatagacakgccaagtttaagatattctaaaatc

SEQ ID NO: 1203 - DNA, 51 bp, Homo sapiens
aaagagggacttctagaatgaccctsagctttctggaactggagtcctttg

SEQ ID NO: 1204 - DNA, 51 bp, Homo sapiens
ctatagaaagactgcaacatcattayaggttttaagagattttattgatat

SEQ ID NO: 1205 - DNA, 51 bp, Homo sapiens
ttataaggaaagctcactttagcaayagagctgacacagtagtagcaggta

SEQ ID NO: 1206 - DNA, 51 bp, Homo sapiens
acattcctggggaaaattggcacttrtgaaagtggagctgtgagtttcttt

SEQ ID NO: 1207 - DNA, 51 bp, Homo sapiens
gatctgcaagttggcatcaggaaaakaaattctgtagtactctgtattaac

SEQ ID NO: 1208 - DNA, 51 bp, Homo sapiens
gctcatacgacctctgctttacaaartgtattacaaaggggagagagggtc

SEQ ID NO: 1209 - DNA, 51 bp, Homo sapiens
ctagtgaccctgccatgttactacakattttgaatatcatctcaacctgga

SEQ ID NO: 1210 - DNA, 51 bp, Homo sapiens
aaatgtttaagaaaaaataaaggagkagaaatctatagatcaaaacaatct

SEQ ID NO: 1211 - DNA, 51 bp, Homo sapiens
ggagtccttcaatccatgaatctaayacatttctacattgatttgagtctt

SEQ ID NO: 1212 - DNA, 51 bp, Homo sapiens
gctacattatggtggcagtaacagakgtttatttttcattcactctacgcg

SEQ ID NO: 1213 - DNA, 51 bp, Homo sapiens
aagagtagttaaccttctactatgayatcccagaaggtaagtctgaatcaa

SEQ ID NO: 1214 - DNA, 51 bp, Homo sapiens
aatagccaatatatagcatttattakgagtaagacactccttcaagcattt

SEQ ID NO: 1215 - DNA, 51 bp, Homo sapiens
ttcacttcaaggatttaagagaccakcgaactttttctctgtggtagaaag

SEQ ID NO: 1216 - DNA, 51 bp, Homo sapiens
ccctttagccttgtgttggatcaccyagttgtaatctaagcttctacacag

SEQ ID NO: 1217 - DNA, 51 bp, Homo sapiens
ataatttctagtttaactattaaaarcaatctggtgttgtcaacctacgtt

SEQ ID NO: 1218 - DNA, 51 bp, Homo sapiens
agtgcctgctaagtcctagagtggtraataaaagagacagggctcctcagg

SEQ ID NO: 1219 - DNA, 51 bp, Homo sapiens
atttgtgactaccaattagctctcaygtttccatagactgttaggacacct

SEQ ID NO: 1220 - DNA, 51 bp, Homo sapiens
tagaaaaataagctgtctgcacttamgtgttggtagttttcatcataaaaa

SEQ ID NO: 1221 - DNA, 51 bp, Homo sapiens
ggattagttgggaaattcctctgaartgtgtgcattatgtctgactttggt

SEQ ID NO: 1222 - DNA, 51 bp, Homo sapiens
tttggaagttcctaaggaatctatcrccagcttacaaagagctgcctttct

SEQ ID NO: 1223 - DNA, 51 bp, Homo sapiens
ttgattcttgctctgaatgataaaartgatgaccgctaaacaggtttgttt

SEQ ID NO: 1224 - DNA, 51 bp, Homo sapiens
caagtatttgctaatggaattcaacragggaatactaggggaagtaagttg

SEQ ID NO: 1225 - DNA, 51 bp, Homo sapiens
cagtttaattgggtagttatatttgwagactatgcaatagtaagtcctatc

SEQ ID NO: 1226 - DNA, 51 bp, Homo sapiens
tgtacttagattgtgttgctgagaartcaaaggtctcttcaacttcagcat

SEQ ID NO: 1227 - DNA, 51 bp, Homo sapiens
gagtacctgaaaaaacacatttccayaataagacaactggttgttcaagac

SEQ ID NO: 1228 - DNA, 51 bp, Homo sapiens
gaccacattttgagaaccactggtayggaggaagtaaactacttgagactg

SEQ ID NO: 1229 - DNA, 51 bp, Homo sapiens
ctagaagatcatactgtttctattcsaactcacaattggaggactggattg

SEQ ID NO: 1230 - DNA, 51 bp, Homo sapiens
tgaaattatttttttaaatacaaagwactatgcaaatataaagaaatgtta

SEQ ID NO: 1231 - DNA, 51 bp, Homo sapiens
cgaaaatattagaagttacagatacrcctcacagtatatttttattaaaca

SEQ ID NO: 1232 - DNA, 51 bp, Homo sapiens
atggaaggcctgctaaatatatcccyggagtgctttgctttctaagggcaa

SEQ ID NO: 1233 - DNA, 51 bp, Homo sapiens
aaggagagaaaggaattttataccayatgagacattagtaaagatcttgga

SEQ ID NO: 1234 - DNA, 51 bp, Homo sapiens
ctgtactgtagaaactagcctaaaaygtattgtcaaagggtcagtgtaaaa

SEQ ID NO: 1235 - DNA, 51 bp, Homo sapiens
acgaattataaataaacatgcaaaamagtttatgtaagttagagcaaaaag

SEQ ID NO: 1236 - DNA, 51 bp, Homo sapiens
taccagcataaagtagcacaatttayacacaatctaaagctctaaggaatg

SEQ ID NO: 1237 - DNA, 51 bp, Homo sapiens
ggggccaggttgagaggagctgtgcraatgaatatgaagcccacttccttc

SEQ ID NO: 1238 - DNA, 51 bp, Homo sapiens
gatttgggagaaaatttaaaaagatkatacatgtgacaaacttaccaaggt

SEQ ID NO: 1239 - DNA, 51 bp, Homo sapiens
ccctctctttctaattttcagccacrtttatttactgtctgtttctccctc

SEQ ID NO: 1240 - DNA, 51 bp, Homo sapiens
caaagcatgctcccttgtgaaaaaayattaagttaccagctggcacaaatt

SEQ ID NO: 1241 - DNA, 51 bp, Homo sapiens
ctgagcttactgaagccaattatccmaagagtatgtgatccagaagcaaga

SEQ ID NO: 1242 - DNA, 51 bp, Homo sapiens
tttgtcatacttcactgtattcacayacttcttttggatgactttgaaatt

SEQ ID NO: 1243 - DNA, 51 bp, Homo sapiens
ccctccaaatcgaatcattgacattrcatatgcccagtagtgtattagctc

SEQ ID NO: 1244 - DNA, 51 bp, Homo sapiens
tagatgcggtttcaggttttaaatarctatgatgattgacgcatgattaaa

SEQ ID NO: 1245 - DNA, 51 bp, Homo sapiens
gctgggagacttactgaaacatatgraaatgctagtgctctgcgttttgaa

SEQ ID NO: 1246 - DNA, 51 bp, Homo sapiens
ttgtgacctctcaccctgagctgcasaccttctgtttcgtgatccgcgtgc

SEQ ID NO: 1247 - DNA, 51 bp, Homo sapiens
aacctatctaccactcattgctctcmaggactatctatgggctcgtagtcc

SEQ ID NO: 1248 - DNA, 51 bp, Homo sapiens
ctacatgtggacctccgtgagaaaasgcatatgcataggacggtgagcagt

SEQ ID NO: 1249 - DNA, 51 bp, Homo sapiens
ctattttgatttataaatggacaggratatatcagtaagacactaccagag

SEQ ID NO: 1250 - DNA, 51 bp, Homo sapiens
cctctgaaatgtgtgcattatgtctkactttggtcttagttgggtcacagg

SEQ ID NO: 1251 - DNA, 51 bp, Homo sapiens
ttttatgccaaggcccaccaaagaayctgcacatacattaagcccaataaa

SEQ ID NO: 1252 - DNA, 51 bp, Homo sapiens
gtgcagtctaaggctctccctatgaygtctgtaaattcccaggaggattct

SEQ ID NO: 1253 - DNA, 51 bp, Homo sapiens
ccaaataaagccagctgcctatttcrcacatacagaagacatacaacttcc

SEQ ID NO: 1254 - DNA, 51 bp, Homo sapiens
tcatagggagagccttagactgcacmaatcgtagaatgggtttagctagtc

SEQ ID NO: 1255 - DNA, 51 bp, Homo sapiens
actcactgaacaacaaaagctgcaayataaccatatcagtggtgatatctt

SEQ ID NO: 1256 - DNA, 51 bp, Homo sapiens
ttctggttctctgaaggaccctaackaatacacctcccctccctcattctc

SEQ ID NO: 1257 - DNA, 51 bp, Homo sapiens
agatctgtcccactgctgatgctaartacgtcaaaactaagtcttattctg

SEQ ID NO: 1258 - DNA, 51 bp, Homo sapiens
ccttcatgccactagacctagagagyacctggctataggaatgctttcttc

SEQ ID NO: 1259 - DNA, 51 bp, Homo sapiens
aaagtatgttttatcaaatatgattrgaaaacaatgcattatgtatccaac

SEQ ID NO: 1260 - DNA, 51 bp, Homo sapiens
ttctacatctatgctttcaaagtaawacactgatatatcatttcatctcca

SEQ ID NO: 1261 - DNA, 51 bp, Homo sapiens
aatacttacagctggttatggaaatsaagattcaactggtcccctatgttg

SEQ ID NO: 1262 - DNA, 51 bp, Homo sapiens
cattattgaggcagctcatgtacacwttttttttgggttggtattctttat

SEQ ID NO: 1263 - DNA, 51 bp, Homo sapiens
ggccccaggcaggtaaatcctactayggaatggagagctcagactctagaa

SEQ ID NO: 1264 - DNA, 51 bp, Homo sapiens
gagttaatggagcataatctcatttrcaatctctgagaaccacatagattt

SEQ ID NO: 1265 - DNA, 51 bp, Homo sapiens
tggccaccatgaacccaagatctagrcctgctccagtggacacaggctcca

SEQ ID NO: 1266 - DNA, 51 bp, Homo sapiens
aaatatatggtcaatttatttttaawgtttcatatgtgcttcaaagtatgg

SEQ ID NO: 1267 - DNA, 51 bp, Homo sapiens
agacaaatttatgcaattctgagacrgcgttagctcatggtatctcctaat

SEQ ID NO: 1268 - DNA, 51 bp, Homo sapiens
atttttctaacattttcggtttggcrtagcacattgtgccaacattccttt

SEQ ID NO: 1269 - DNA, 51 bp, Homo sapiens
acttaccttttcttctaccatggcakgaaccttgcaccattgttggtgctt

SEQ ID NO: 1270 - DNA, 51 bp, Homo sapiens
aaactctgcagcttacgtttggataktagacatctaatgacttggctgact

SEQ ID NO: 1271 - DNA, 51 bp, Homo sapiens
tatgacagagcacctaatataatcamtaaatgacacatgacaccataccta

SEQ ID NO: 1272 - DNA, 51 bp, Homo sapiens
aaatgtttacttactctatggtgcayactaagttaaaaaagaggtagaaat

SEQ ID NO: 1273 - DNA, 51 bp, Homo sapiens
gggggtagcaggaaaaactagcccayagccagagatggtaccataaggagg

SEQ ID NO: 1274 - DNA, 51 bp, Homo sapiens
gagcagcaaccagaattctaatctcyactgttgcaagctcaggctccatgc

SEQ ID NO: 1275 - DNA, 51 bp, Homo sapiens
tggacaattttgttgtctaaattaaygtagagcaaggttttaaaactatgg

SEQ ID NO: 1276 - DNA, 51 bp, Homo sapiens
accacctgccaaggcatacaatccgsacaaagtaaaccatcataatttcaa

SEQ ID NO: 1277 - DNA, 51 bp, Homo sapiens
tagttcctggatctcacactgaatcmaatatatgctccagggttatactaa

SEQ ID NO: 1278 - DNA, 51 bp, Homo sapiens
aagctaaacataaagacctaaaatayaccaacagaactaagagcatattaa

SEQ ID NO: 1279 - DNA, 51 bp, Homo sapiens
attgaaagcttattatgtgtcactaycttgtacctcttaaaattactgaaa

SEQ ID NO: 1280 - DNA, 51 bp, Homo sapiens
gatttgtgtttgatgtcaataaactwaatgagacacaatcttcccctcaag

SEQ ID NO: 1281 - DNA, 51 bp, Homo sapiens
tttttgttgattatgattaaacaatmaaagtaaagtctaactcagggtaga

SEQ ID NO: 1282 - DNA, 51 bp, Homo sapiens
atcatttagagatcatcagaaacaarttaagtttgaatatgaaagcttccc

SEQ ID NO: 1283 - DNA, 51 bp, Homo sapiens
gttgtacagatttctcatttacagtkaagttcatgaatcatttccagttca

SEQ ID NO: 1284 - DNA, 51 bp, Homo sapiens
gtaattaacagtttacattactagascaattatttccatttttatatattt

SEQ ID NO: 1285 - DNA, 51 bp, Homo sapiens
actctccttagtctgtggaaaacagrgattgtatcttatgtacatttcacc

SEQ ID NO: 1286 - DNA, 51 bp, Homo sapiens
tatatgtcattcattcattccaggarcttctactgagcagttatcatgcca

SEQ ID NO: 1287 - DNA, 51 bp, Homo sapiens
gtgctggtccaggttttccaagaaayagatgttgagatgggattttatcag

SEQ ID NO: 1288 - DNA, 51 bp, Homo sapiens
tgaccatcttttctacaatgttcaarttaaaatcaaaaggttattgaactt

SEQ ID NO: 1289 - DNA, 51 bp, Homo sapiens
atttgtacatgtttaatacagagaartcaactgagccagatgcacacagag

SEQ ID NO: 1290 - DNA, 51 bp, Homo sapiens
ttttctaatgggtcttaagactatamaactctaataggcccacattttctc

SEQ ID NO: 1291 - DNA, 51 bp, Homo sapiens
gtcttcttcaccctcatgtctgatcratgaccatgggttgagagattttat

SEQ ID NO: 1292 - DNA, 51 bp, Homo sapiens
actgactctccaggcaggtcttaaasatatgcttcacttgaccaatttgtt

SEQ ID NO: 1293 - DNA, 51 bp, Homo sapiens
atgcataaaaatagccgtgagagaartaacaggtggaaggatcaggagtga

SEQ ID NO: 1294 - DNA, 51 bp, Homo sapiens
tcttctacctggctttgttctatcayccgtatctgctaagatgttactgct

SEQ ID NO: 1295 - DNA, 51 bp, Homo sapiens
ctttagaaagacagaattttcaatcyattggctgttgtattttaagaagtt

SEQ ID NO: 1296 - DNA, 51 bp, Homo sapiens
ccctggtacagacctaaaggaccacsatcaaatatccattgttaagtactt

SEQ ID NO: 1297 - DNA, 51 bp, Homo sapiens
gctgtagctcatcctatgctggaaartagagaaaatgtgaggtctcctgca

SEQ ID NO: 1298 - DNA, 51 bp, Homo sapiens
tgtcaaaaactgttacctctttttargtacttgtctttgtgaatcaaaacg

SEQ ID NO: 1299 - DNA, 51 bp, Homo sapiens
ctgctttcagctttatttttcctaayttaactttcaggtatcatcatcttt

SEQ ID NO: 1300 - DNA, 51 bp, Homo sapiens
agaaacaccttttaatcactatttcycttgtaagtgacacttataaatccc

SEQ ID NO: 1301 - DNA, 51 bp, Homo sapiens
caaacaaacaaatattccatgttagraagataaccattagagtaatcctgc

SEQ ID NO: 1302 - DNA, 51 bp, Homo sapiens
tttggcagtaattgactgaaatgaayctttctaaactttacagtactaaag

SEQ ID NO: 1303 - DNA, 51 bp, Homo sapiens
aacatgttagtcttcatgtgatgacygggaaatttaagagcactccagctc

SEQ ID NO: 1304 - DNA, 51 bp, Homo sapiens
aattcctaagataataacaccttccrcatacatttaggacactcaatcaca

SEQ ID NO: 1305 - DNA, 51 bp, Homo sapiens
gatgatgaggagagaaaacacaggasagccaggccttcttggtttatgcag

SEQ ID NO: 1306 - DNA, 51 bp, Homo sapiens
gtgagtccaaacggtttcaaagcccraaggctgtgtgagttgtagaatgtg

SEQ ID NO: 1307 - DNA, 51 bp, Homo sapiens
tgtttggctcgcttgagacaaaggcygatgtctgattcttacataaatgct

SEQ ID NO: 1308 - DNA, 51 bp, Homo sapiens
aatttccaccttctccattgatgtaratttgacaggttgtatatgtaaaag

SEQ ID NO: 1309 - DNA, 51 bp, Homo sapiens
aactgatgttattgttgaattagtaragggacagacatcctgttgtgtgaa

SEQ ID NO: 1310 - DNA, 51 bp, Homo sapiens
tcatgggaaaatcggcatgtaacatsagtcttttggtattgaggtctcatt

SEQ ID NO: 1311 - DNA, 51 bp, Homo sapiens
aactagaatgatctgtgtgattctargtaagtcttataatcttgctgttcc

SEQ ID NO: 1312 - DNA, 51 bp, Homo sapiens
ttaagtaaaacagacataatagtaaygatagcagctacttcatagagtctt

SEQ ID NO: 1313 - DNA, 51 bp, Homo sapiens
gctgagcataaaactcctttctccaytccttcatttattaactcccccctt

SEQ ID NO: 1314 - DNA, 51 bp, Homo sapiens
acaggttctatttttgctctataaaytgatagctctttgttcatcaaaagc

SEQ ID NO: 1315 - DNA, 51 bp, Homo sapiens
caaaaacagctatatgttatctccartctactctcataccagcttttcttt

SEQ ID NO: 1316 - DNA, 51 bp, Homo sapiens
gagataaatgctcacccactttaaaraagaaatcgataattgtaattcact

SEQ ID NO: 1317 - DNA, 51 bp, Homo sapiens
atttaaacttttatatacttagcgayattttatcccctttgacaagcgaat

SEQ ID NO: 1318 - DNA, 51 bp, Homo sapiens
gcacatcttttacatctacaaagtasagatgatcatggtaatgccaacctc

SEQ ID NO: 1319 - DNA, 51 bp, Homo sapiens
aattatattatattgaccatattatraattttgcatcattgtcgcctccta

SEQ ID NO: 1320 - DNA, 51 bp, Homo sapiens
cggcagtatatgaattctaaccacaygtatggtaaccatggacttcacaat

SEQ ID NO: 1321 - DNA, 51 bp, Homo sapiens
caccatgactgagagaattagctgaygaaggaagggcttagcagttgctta

SEQ ID NO: 1322 - DNA, 51 bp, Homo sapiens
gcaagcttgagaaaaaacctggatayggaagactggatggaccaggagaag

SEQ ID NO: 1323 - DNA, 51 bp, Homo sapiens
atcataaataataagaaggaaaacaygatggtgaccatagattctaaacta

SEQ ID NO: 1324 - DNA, 51 bp, Homo sapiens
atgacaaccaagcacacatttgatastacagtactaagaaagatttggggc

SEQ ID NO: 1325 - DNA, 51 bp, Homo sapiens
aattgaaatggctgctctgacacacrctacaaagttggaacctgatgcaat

SEQ ID NO: 1326 - DNA, 51 bp, Homo sapiens
agtgacagaagaaacactaaattcaygatgataattgcctatggagaggag

SEQ ID NO: 1327 - DNA, 51 bp, Homo sapiens
gttctgtaatgaaagtgtatctttamctcattatttatttattttattgag

SEQ ID NO: 1328 - DNA, 51 bp, Homo sapiens
tgaaagtatttacatgcgtttcatartaaattccggccaagctgaagtaaa

SEQ ID NO: 1329 - DNA, 51 bp, Homo sapiens
aaggtgtgaaaaatgaaagaaatccractgctagccagatttattcttttc

SEQ ID NO: 1330 - DNA, 51 bp, Homo sapiens
gtatttgtaaacacattttctcataycttaaaaaacgcaggagctgataag

SEQ ID NO: 1331 - DNA, 51 bp, Homo sapiens
tagccacagagggattgatgtttacygcaaagagcctggagtcagaagatc

SEQ ID NO: 1332 - DNA, 51 bp, Homo sapiens
tctatccttgtgactggcaccttcawctatctactgcccaagtcagaaaat

SEQ ID NO: 1333 - DNA, 51 bp, Homo sapiens
acatgtatacatgtatttctggttcscaatgatcttgaccaagagagtatc

SEQ ID NO: 1334 - DNA, 51 bp, Homo sapiens
aattacggcctacgagttcacaaaayttagcacagaatgctaaacagttgt

SEQ ID NO: 1335 - DNA, 51 bp, Homo sapiens
cccacaagataaagttcccaaggcamtatgacccctgattccttctctatc

SEQ ID NO: 1336 - DNA, 51 bp, Homo sapiens
cttatgagattatgagaaattctgasattatgagaaatctgtatattgtga

SEQ ID NO: 1337 - DNA, 51 bp, Homo sapiens
acaggaagtagctatgtgtcttgcamcaggaatgatacgaaggttctaatc

SEQ ID NO: 1338 - DNA, 51 bp, Homo sapiens
ggtgatatattaattaattgacaacraatggtacaagcaatacatagcatg

SEQ ID NO: 1339 - DNA, 51 bp, Homo sapiens
ggctgttcagtattcttttgccagarcataccacaattattaatccgttca

SEQ ID NO: 1340 - DNA, 51 bp, Homo sapiens
gccccttgaaaaactctagagtacarattcacattactcagattgaataag

SEQ ID NO: 1341 - DNA, 51 bp, Homo sapiens
ctttccatccttatgatggttcttayggtcttcaaatttgctgcttatttc

SEQ ID NO: 1342 - DNA, 51 bp, Homo sapiens
ctatcttcataaatttttatccccaygagctaattgttttctccttcattt

SEQ ID NO: 1343 - DNA, 51 bp, Homo sapiens
agcttgagagccaagtcataacttakcaagaacaattccacggttactata

SEQ ID NO: 1344 - DNA, 51 bp, Homo sapiens
aagaccttcctcaatttgataatacracaagctagccatgtgaaaattgac

SEQ ID NO: 1345 - DNA, 51 bp, Homo sapiens
ctagctctaaggagtagttccctaaygcagctgtagcttttggagtttgaa

SEQ ID NO: 1346 - DNA, 51 bp, Homo sapiens
actttttcagctggagatcttgataygagaaattaatggatattgattcaa

SEQ ID NO: 1347 - DNA, 51 bp, Homo sapiens
aagagaattacctgaaaaacaagaargtagtcagatataagattttaaaaa

SEQ ID NO: 1348 - DNA, 51 bp, Homo sapiens
gagcaatttcttatatataaaagccratacagatgtgaacacatacataca

SEQ ID NO: 1349 - DNA, 51 bp, Homo sapiens
caagttgccaaggggtctcgacttayagcaatctttcaaaggcctaggatg

SEQ ID NO: 1350 - DNA, 51 bp, Homo sapiens
tattattgagcacacattcagtacayagcaatttaatagtcactgagaatt

SEQ ID NO: 1351 - DNA, 51 bp, Homo sapiens
taccaggctagtacctgagaaataasctgatttgggaaataaaatcttacc

SEQ ID NO: 1352 - DNA, 51 bp, Homo sapiens
aaatttgtattttattatctatctgkatctaggagccttactataaattat

SEQ ID NO: 1353 - DNA, 51 bp, Homo sapiens
aaacccaggagcaaatttatcacaamgaggtttattaaacttgtaaactcc

SEQ ID NO: 1354 - DNA, 51 bp, Homo sapiens
tcagagaggggatgcaatgccttgawgagttacagaaagagagagattgag

SEQ ID NO: 1355 - DNA, 51 bp, Homo sapiens
acttttatttctcaaggagcagatastagatatacataccatgttgactca

SEQ ID NO: 1356 - DNA, 51 bp, Homo sapiens
acaagttaatcaaaataaatcgaaayggcgcatgcctgggtaacactcttc

SEQ ID NO: 1357 - DNA, 51 bp, Homo sapiens
gtcaaaatttcagtcttggtaagagyattatcagcttgtattattcattaa

SEQ ID NO: 1358 - DNA, 51 bp, Homo sapiens
atagcaaggctgcaaaggaaaggtarctggtgcaaatggttgcacgggaac

SEQ ID NO: 1359 - DNA, 51 bp, Homo sapiens
cttctttgataattcgatgtgtccasctagggcctcacacagggatttgtg

SEQ ID NO: 1360 - DNA, 51 bp, Homo sapiens
atttcataaattaacaacagaagatkaattagagaagaaaaatagcctata

SEQ ID NO: 1361 - DNA, 51 bp, Homo sapiens
tcaaaaggacttcgccaaaaataackatacaagtgagaggatttctaacat

SEQ ID NO: 1362 - DNA, 51 bp, Homo sapiens
aactgtgattcaacactgagtgctcrcttggaaaggaggtggagctcaact

SEQ ID NO: 1363 - DNA, 51 bp, Homo sapiens
ttaaaaaagagtacctgaaatggaartcatactatgaggttttagtttcta

SEQ ID NO: 1364 - DNA, 51 bp, Homo sapiens
caaaaatgggacagtttgaccagcaytataacctatttctccaccagaaaa

SEQ ID NO: 1365 - DNA, 51 bp, Homo sapiens
aaaaattaatccatccagactcatamcttaagagaatattttataaaagga

SEQ ID NO: 1366 - DNA, 51 bp, Homo sapiens
tatttaactaattccctattgatggrcattaaaatacttctgtaactattg

SEQ ID NO: 1367 - DNA, 51 bp, Homo sapiens
tgtgtgtgtgtgtgtgtttaattcakagcaacctgttacattctttctatt

SEQ ID NO: 1368 - DNA, 51 bp, Homo sapiens
ggcacagaaatcagcaaacggtacaygccagggccacccctcccagacagc

SEQ ID NO: 1369 - DNA, 51 bp, Homo sapiens
atgtatgcatcagaggccttcaaacsagggtgtctatcctcctggggtaca

SEQ ID NO: 1370 - DNA, 51 bp, Homo sapiens
aaagacttttggaaatcatttagacwacatttaatgtctctcctgctctct

SEQ ID NO: 1371 - DNA, 51 bp, Homo sapiens
cctatgaaacgcgcctgtaacagcgycaagaacagtcaacacagcactaga

SEQ ID NO: 1372 - DNA, 51 bp, Homo sapiens
tagcaagcttgaaggagcagttacayacgaaagaacttgcttaacaagtgg

SEQ ID NO: 1373 - DNA, 51 bp, Homo sapiens
cagagtgatactttgaaatgtaatcraatgaagagagctttaacataccca

SEQ ID NO: 1374 - DNA, 51 bp, Homo sapiens
tctgttgagcaaagtagtgtaaaaayggatcatcttttctaccaattttat

SEQ ID NO: 1375 - DNA, 51 bp, Homo sapiens
agtttagttcaaacattgtattaaawattatccatgaaacagtcgctgggc

SEQ ID NO: 1376 - DNA, 51 bp, Homo sapiens
ccatccagaatcaaaaccaaaacacyctactcaactgacactctagacata

SEQ ID NO: 1377 - DNA, 51 bp, Homo sapiens
aacaaaaatgtatgactcttatttaycattttgcaattagttgatcataac

SEQ ID NO: 1378 - DNA, 51 bp, Homo sapiens
ggaaataatgcatgtaaattcttacyatggtggctagaacactgaaggatt

SEQ ID NO: 1379 - DNA, 51 bp, Homo sapiens
atgaacctacacaacgttttacgaaygtccagacttctccataataggcca

SEQ ID NO: 1380 - DNA, 51 bp, Homo sapiens
caaatctacagccatgcaacactaargctgaaaatttaaatgcataagtta

SEQ ID NO: 1381 - DNA, 51 bp, Homo sapiens
atacatgctaacttcagcctcctaayatagtgaagactggatgaaatacat

SEQ ID NO: 1382 - DNA, 51 bp, Homo sapiens
cccgagagccaccaggctaaagaaasagcagtagcttcaagcgtatgaatt

SEQ ID NO: 1383 - DNA, 51 bp, Homo sapiens
cacgaccgtcaatttactcatctacrgaatggaagaaaatcaggtaactct

SEQ ID NO: 1384 - DNA, 51 bp, Homo sapiens
tattgttctaagcccaatcacttcarataaaattatgatgccacattctga

SEQ ID NO: 1385 - DNA, 51 bp, Homo sapiens
aaaatcatcactaagacaagaagaayaaacacctgaggaacaagagtcctc

SEQ ID NO: 1386 - DNA, 51 bp, Homo sapiens
aagcttaattcacaaggagttgtcaygtgctctgggtgaactaggagcata

SEQ ID NO: 1387 - DNA, 51 bp, Homo sapiens
tagattctgacagaattgccctgtayatccatgtgtcaacatgaaaggggt

SEQ ID NO: 1388 - DNA, 51 bp, Homo sapiens
aatgaatgagtgttgtgatcaaatartcctcacgtatagggtgtgccaagg

SEQ ID NO: 1389 - DNA, 51 bp, Homo sapiens
acatctcttagcatccagcgaaaatrataatgcttaaaaaaaaaagaaaat

SEQ ID NO: 1390 - DNA, 51 bp, Homo sapiens
tggaaagtgatcattggaaagacacrataaagagaatagggaaagcaaaaa

SEQ ID NO: 1391 - DNA, 51 bp, Homo sapiens
tttagatattatagattccaaaaaayttaacagagagtttgtggtggtgtc

SEQ ID NO: 1392 - DNA, 51 bp, Homo sapiens
agtagaattcaagttcacagaatacycaaactaaggtgagtgatcagacag

SEQ ID NO: 1393 - DNA, 51 bp, Homo sapiens
taatcagaactaaaatcataaacacrggtataggcttttataggtgatacc

SEQ ID NO: 1394 - DNA, 51 bp, Homo sapiens
gaaatgtaaataattttgtgatgcaktatcaacattacttgtttatgatta

SEQ ID NO: 1395 - DNA, 51 bp, Homo sapiens
gctattaaataccaaatttccacacrggagaatgatctcagttaatgcctc

SEQ ID NO: 1396 - DNA, 51 bp, Homo sapiens
agataagcacacaaatgagattgggyaatgctatcaagggaggagaaagac

SEQ ID NO: 1397 - DNA, 51 bp, Homo sapiens
tcatcccccaaatttaggttttcaamaatcaagcagaaaaggaggctccta

SEQ ID NO: 1398 - DNA, 51 bp, Homo sapiens
caatcagattctatgggaatgacagmaaattaaattgtataaccaagtcct

SEQ ID NO: 1399 - DNA, 51 bp, Homo sapiens
ctttgagagttaaccctttctgtaartgcataatgtctccagaagctttca

SEQ ID NO: 1400 - DNA, 51 bp, Homo sapiens
gtggacctttgcacttgcgtacttaygtaaaactgccgctcaggagagtct

SEQ ID NO: 1401 - DNA, 51 bp, Homo sapiens
aagttatgaagtttaagagtatgcartaggaaagattcagagaaagagcaa

SEQ ID NO: 1402 - DNA, 51 bp, Homo sapiens
tgtattttaccatacctctctcttckcagaagccatcttctgatctttttt

SEQ ID NO: 1403 - DNA, 51 bp, Homo sapiens
ttggattttaagctacttaaatgcamataaatgattttaaaagtatgagtt

SEQ ID NO: 1404 - DNA, 51 bp, Homo sapiens
ctttgagcacttcaaatgacacttckatatttgtaccttacacaaaggata

SEQ ID NO: 1405 - DNA, 51 bp, Homo sapiens
ctcctttcccctgcattgaggtagcyctttaaattacaagattttagacag

SEQ ID NO: 1406 - DNA, 51 bp, Homo sapiens
tgtacaaatgtgccatttttagtacratagtatccagccttatcagattca

SEQ ID NO: 1407 - DNA, 51 bp, Homo sapiens
tagcaaccatactgataaatacctamacggataaactattaaatggacagg

SEQ ID NO: 1408 - DNA, 51 bp, Homo sapiens
ttagggggataaatttaatctaaaayatgtacttgagaataggcaaagtgt

SEQ ID NO: 1409 - DNA, 51 bp, Homo sapiens
tcagaatagccctctgctggtttgcygattttttattaaataaaggacagt

SEQ ID NO: 1410 - DNA, 51 bp, Homo sapiens
agtgcatccacaaattatatacaaarccaatagttcctatcctgagaatca

SEQ ID NO: 1411 - DNA, 51 bp, Homo sapiens
tgaggcacgatattccatatataaaragccaagaatgactaaataagtttt

SEQ ID NO: 1412 - DNA, 51 bp, Homo sapiens
atttcataattcttaggaatctattsaaaggagaagacatttttttccaca

SEQ ID NO: 1413 - DNA, 51 bp, Homo sapiens
atttctcttattctgtttttacaaartatgttctacatttctgtctagtac

SEQ ID NO: 1414 - DNA, 51 bp, Homo sapiens
gaaaaacccaaagatatggtacaaarcaatttataaagacagaaacttatg

SEQ ID NO: 1415 - DNA, 51 bp, Homo sapiens
agggcaaggcagaggccaaagtccaygcaaagttccagactgaagagcaac

SEQ ID NO: 1416 - DNA, 51 bp, Homo sapiens
ttaaaagtaaacatttggattgccaygattctgattataaatggataaaat

SEQ ID NO: 1417 - DNA, 51 bp, Homo sapiens
caacagctattttattcttgacatcratttctgaaaacatcacctgtgaaa

SEQ ID NO: 1418 - DNA, 51 bp, Homo sapiens
tttactgctctaccagtagcagtaamgattatgcaaactcaaaagctaaat

SEQ ID NO: 1419 - DNA, 51 bp, Homo sapiens
tgatgatttgcaaatatagtcaaatraaagtgcatttctaaatggaactga

SEQ ID NO: 1420 - DNA, 51 bp, Homo sapiens
ttgactggtggatcacagagactcasaggctaaaggaatgagataattcca

SEQ ID NO: 1421 - DNA, 51 bp, Homo sapiens
gcagccacctctaacctaagcattgycaacattataattgtaagtcgtcta

SEQ ID NO: 1422 - DNA, 51 bp, Homo sapiens
gtcctggagactgtttcaaatcccayagtgagagaagatgaaacctcttgt

SEQ ID NO: 1423 - DNA, 51 bp, Homo sapiens
gaagggagttgcatgcattaagtcayagaggcctgaacaaagaatgtggtg

SEQ ID NO: 1424 - DNA, 51 bp, Homo sapiens
tgaaaaatgcactgggcttgcaaacrcttttgtcatgtcaacacaactgag

SEQ ID NO: 1425 - DNA, 51 bp, Homo sapiens
agctatgtttatggatttaccatgargaatagataactaaagatacacttt

SEQ ID NO: 1426 - DNA, 51 bp, Homo sapiens
ttcacaaaatcaaagtatctagcacygaccctatgaacaaatgagcatgaa

SEQ ID NO: 1427 - DNA, 51 bp, Homo sapiens
ctcaagagaagcaaaaatagtaaaawttggtttatgctgctttagcaatat

SEQ ID NO: 1428 - DNA, 51 bp, Homo sapiens
atattccaaatgccttgcatcaagamgtcaaaaactctgctcagcccactc

SEQ ID NO: 1429 - DNA, 51 bp, Homo sapiens
cagaaccaaaacacatgcctgaagastatgcatttaaggagtcatttctgt

SEQ ID NO: 1430 - DNA, 51 bp, Homo sapiens
tggtactgactccttgattcatgtasgaaacatgtttggctatctgtaaaa

SEQ ID NO: 1431 - DNA, 51 bp, Homo sapiens
ggctatggatgtctagaatgaaagayggattgtagataacagtcttgtcat

SEQ ID NO: 1432 - DNA, 51 bp, Homo sapiens
agcttaaaatataaaataatttgggmagattgttagaaacaaggtgtgcta

SEQ ID NO: 1433 - DNA, 51 bp, Homo sapiens
tctagaaaatcatatttacaaatcaygccgcttcagtgggtataaagaaac

SEQ ID NO: 1434 - DNA, 51 bp, Homo sapiens
aatgagacatcttatttcgatgaagscatctacttgaagttactggaaaaa

SEQ ID NO: 1435 - DNA, 51 bp, Homo sapiens
ccttatcgagaaatgcaggtctgtarcggtgagataaggtctctctagctg

SEQ ID NO: 1436 - DNA, 51 bp, Homo sapiens
tgctaagaatttatccttatgcccaygcggtttttggatgactggcatttt

SEQ ID NO: 1437 - DNA, 51 bp, Homo sapiens
taatgtctgggattaaacctaatacygaggaataccttgtccacccaagct

SEQ ID NO: 1438 - DNA, 51 bp, Homo sapiens
catatcatgatctcaccaaattcacsagtttactgtaatttggaaacatac

SEQ ID NO: 1439 - DNA, 51 bp, Homo sapiens
aaataatttcctaagaacataaatawctaaagacaaatgagcatatcacaa

SEQ ID NO: 1440 - DNA, 51 bp, Homo sapiens
ttccccaggatatggtagattaaacycagtcaccaaggctatctgacttgt

SEQ ID NO: 1441 - DNA, 51 bp, Homo sapiens
gaccctatgagaagaggtctctgaayctttggattaaaaaacaacacagcc

SEQ ID NO: 1442 - DNA, 51 bp, Homo sapiens
acgtggagcactgacagtctgagccrcactaggaagctttggcagtcagtc

SEQ ID NO: 1443 - DNA, 51 bp, Homo sapiens
gcaatatgttaaaactggcacggaayctttgaatagcttttgaatttttat

SEQ ID NO: 1444 - DNA, 51 bp, Homo sapiens
attttcctatgactacctgtcttcakaaactaagtgtaagtttgaattctc

SEQ ID NO: 1445 - DNA, 51 bp, Homo sapiens
aatagtttacaaatctatttctccargtcttttctactatcccccaatgca

SEQ ID NO: 1446 - DNA, 51 bp, Homo sapiens
tgatttcattgaacttagagcaaccragaacaagaactgggtttcgtggca

SEQ ID NO: 1447 - DNA, 51 bp, Homo sapiens
ataatagcactgagaacatctgagamagtttgtgcctaataaaattctaga

SEQ ID NO: 1448 - DNA, 51 bp, Homo sapiens
gccagactttttctatcgcctaagamaacagttactatatctgatgttctg

SEQ ID NO: 1449 - DNA, 51 bp, Homo sapiens
aatcagaagtagtatattataatcaraggagttcttggtgtaataggaact

SEQ ID NO: 1450 - DNA, 51 bp, Homo sapiens
acttatcaaatgaaagctataaaagkcaggtctacacaggaaacaatggaa

SEQ ID NO: 1451 - DNA, 51 bp, Homo sapiens
tgtcatgagaaatgagttcagtttgraggattctaaaagtctaggtgaggt

SEQ ID NO: 1452 - DNA, 51 bp, Homo sapiens
ttcagattctgggagcagtttactaygcccttattctcagtcactactgtg

SEQ ID NO: 1453 - DNA, 51 bp, Homo sapiens
acatttctgagcatgtacatcatgcyagaccctgcagcagaaaaaaataaa

SEQ ID NO: 1454 - DNA, 51 bp, Homo sapiens
ttcatcatcgtgtaatagcgcaagaycaccactagatggaagcataggata

SEQ ID NO: 1455 - DNA, 51 bp, Homo sapiens
gtccttgtaggaagggtgattggtasaactttctattctgtcatcttgctc

SEQ ID NO: 1456 - DNA, 51 bp, Homo sapiens
ccccagcctgccaggctggtcacccrggaaggatggataaatattcaagaa

SEQ ID NO: 1457 - DNA, 51 bp, Homo sapiens
atggagaacctcatttcgagtgaaarcactgtccttgatacacacagaaaa

SEQ ID NO: 1458 - DNA, 51 bp, Homo sapiens
cctgacaccgacttcttagagaagckagtcttttttgaatggaggagcgat

SEQ ID NO: 1459 - DNA, 51 bp, Homo sapiens
gggctcatgtttagagagacagataygagtttggaatagctgatctcatga

SEQ ID NO: 1460 - DNA, 51 bp, Homo sapiens
caacagcagcagagacagacagagakttaatccagggtaaggagaacccgg

SEQ ID NO: 1461 - DNA, 51 bp, Homo sapiens
ggtagaggagagacgttctttttttyggttatctgtgatagatggtatgaa

SEQ ID NO: 1462 - DNA, 51 bp, Homo sapiens
atttagttcccctatatattaaacakaacagggcccatgagcagtttttat

SEQ ID NO: 1463 - DNA, 51 bp, Homo sapiens
acatattgttgtgccagccaggcackgaattaagcaacatatcatctcact

SEQ ID NO: 1464 - DNA, 51 bp, Homo sapiens
aatttccgagggagttcctcttggtyctccagacacgcctgctgcaggcct

SEQ ID NO: 1465 - DNA, 51 bp, Homo sapiens
ttctggggctccaggtgagcccttgytttgcccagtctccgtgtctttgtc

SEQ ID NO: 1466 - DNA, 51 bp, Homo sapiens
tagtacattaccataaaatagacccraaagactgatgttttccaaattatg

SEQ ID NO: 1467 - DNA, 51 bp, Homo sapiens
aagccagcagatatactagatatgcraagcaacaattgaaatccatggttt

SEQ ID NO: 1468 - DNA, 51 bp, Homo sapiens
gttcttgtaagaaaggaaatccagayacagacatgtgcaccatgagaggag

SEQ ID NO: 1469 - DNA, 51 bp, Homo sapiens
tatagtttcacgttaatctccagatyctgttaagccactaaataccagttt

SEQ ID NO: 1470 - DNA, 51 bp, Homo sapiens
ttctatggtgactaataccgctgaaycaactcattcaaagatatttttaaa

SEQ ID NO: 1471 - DNA, 51 bp, Homo sapiens
aaatggtgatgaaggtaacaagactragagataccagtctgttgactcaca

SEQ ID NO: 1472 - DNA, 51 bp, Homo sapiens
gcctgtacatttaaatacatgttacrctacttatcacaaggctgaattctg

SEQ ID NO: 1473 - DNA, 51 bp, Homo sapiens
ccttcacccccccaatctgaataagsaaaatgttcatttgcattgatggga

SEQ ID NO: 1474 - DNA, 51 bp, Homo sapiens
ctgctatttcctgcttccctttttcktcagattaattcacaacaatttagc

SEQ ID NO: 1475 - DNA, 51 bp, Homo sapiens
gtacatttgtaggccatcagtggaastgcttgaaaattacagggctccata

SEQ ID NO: 1476 - DNA, 51 bp, Homo sapiens
cacacatttgcatttcaatagcaackcctgggtatgtttaaaaaaatacca

SEQ ID NO: 1477 - DNA, 51 bp, Homo sapiens
ccagggtaccctccttatgacagtaygtttgtaagacaaggacttttaata

SEQ ID NO: 1478 - DNA, 51 bp, Homo sapiens
ctgtttcagtggttgaaccctagctracataaggatactttgtaagtatca

SEQ ID NO: 1479 - DNA, 51 bp, Homo sapiens
cctcccaggaacatgaacatgaaacrcttgagtcccagtgggctgctccgc

SEQ ID NO: 1480 - DNA, 51 bp, Homo sapiens
tggaaatagtttcttcgcctccttcrataccttccaatttaaggtttgatg

SEQ ID NO: 1481 - DNA, 51 bp, Homo sapiens
gaaggtatcgaaggaggcgaagaaastatttccagcagtgaagtgaactag

SEQ ID NO: 1482 - DNA, 51 bp, Homo sapiens
cttggtggtcttcaggtggattcccrgaagctcaagccttcctgagggacc

SEQ ID NO: 1483 - DNA, 51 bp, Homo sapiens
tactcccactttgtcagtgaaactamaaccctctgcttctcacatggcttt

SEQ ID NO: 1484 - DNA, 51 bp, Homo sapiens
ggtaaattaatcttcttctctaaccraaccctgtgtgtatctatccgattt

SEQ ID NO: 1485 - DNA, 51 bp, Homo sapiens
ttatgtattatatataacatgtttakgattaaatctggcccatattctaat

SEQ ID NO: 1486 - DNA, 51 bp, Homo sapiens
aaattggcagaagagctcagatttgragtatatttttaacatagggccaac

SEQ ID NO: 1487 - DNA, 51 bp, Homo sapiens
ttccaattttacagaaaataaatcaygtggcctgcttaaggtcacagagcc

SEQ ID NO: 1488 - DNA, 51 bp, Homo sapiens
atcacttgcccttcttattgctaaargaaccaacagagcaccaaagagacc

SEQ ID NO: 1489 - DNA, 51 bp, Homo sapiens
caacgttctggcttcgtcttataaasaaacatagtgaaagacagaagagcc

SEQ ID NO: 1490 - DNA, 51 bp, Homo sapiens
acaaaaaatgtttgtggttatgcctkaatttactacaacgtatttttgagc

SEQ ID NO: 1491 - DNA, 51 bp, Homo sapiens
tgacaaacaaatccaacaaaagaggyacttagagcagtgttcattgaatcc

SEQ ID NO: 1492 - DNA, 51 bp, Homo sapiens
caaaagcatgagaaatgatacaggaragattgaattgaaagtagcatatcc

SEQ ID NO: 1493 - DNA, 51 bp, Homo sapiens
ggttctagaaactaaatatagaatcrcaatagaatgtagcatataacactt

SEQ ID NO: 1494 - DNA, 51 bp, Homo sapiens
aattcatttcacagaacatggacaaycttaaagagagcagtcaaaatcaga

SEQ ID NO: 1495 - DNA, 51 bp, Homo sapiens
ctaaatacaggttgacatctaccaamaatttaactatctaaaaagaaatgc

SEQ ID NO: 1496 - DNA, 51 bp, Homo sapiens
cgcttttctaaagcagaaatcactamgcatctcagatacctcaagttgcat

SEQ ID NO: 1497 - DNA, 51 bp, Homo sapiens
tacagcttggatttcttgtgccaaaygatcaaacagtaaaagcactgacaa

SEQ ID NO: 1498 - DNA, 51 bp, Homo sapiens
agcaagaaactaactggtgggagtaragtgtgaagcacaaacgtgctaaat

SEQ ID NO: 1499 - DNA, 51 bp, Homo sapiens
aaaaaaaaagaacctgattaagttarcacgtggcacttaaactgaagtata

SEQ ID NO: 1500 - DNA, 51 bp, Homo sapiens
tctatgtactcatgttttgataaggraaataaaatattcccagtattctta

SEQ ID NO: 1501 - DNA, 51 bp, Homo sapiens
cctgatgttcaaatccaccgataagsaagttcatcatttactgtgctatat

SEQ ID NO: 1502 - DNA, 51 bp, Homo sapiens
ctatttcagcagacagagacagtgasagaccttgtgggtgggaggagggag

SEQ ID NO: 1503 - DNA, 51 bp, Homo sapiens
agccattaattgatactcagatttgrtataagaggacagaaggaatggcag